(19) United States
(12) Patent Application Publication
(10) Pub. No.: US 2026/0339202 A1
(43) Pub. Date: Oct. 29, 2026

(54) ONIUM SALT MONOMER, MONOMERIC PHOTO-ACID GENERATOR, POLYMER, CHEMICALLY-AMPLIFIED RESIST COMPOSITION, AND PATTERNING PROCESS

(71) Applicant: SHIN-ETSU CHEMICAL CO., LTD., Tokyo (JP)

(72) Inventors: Ryo SUDO, Joetsu-shi (JP); Sotaro TAKAHASHI, Joetsu-shi (JP); Gentaro HIDA, Joetsu-shi (JP)

(73) Assignee: SHIN-ETSU CHEMICAL CO., LTD., Tokyo (JP)

(21) Appl. No.: 19/643,314

(22) Filed: Apr. 9, 2026

(30) Foreign Application Priority Data
Apr. 15, 2025 (JP) 2025-067147

(51) Int. Cl. G03F 7/004; C07C 309/42; C07C 309/49; C07C 323/64; C07D 333/76; C08F 220/18; C08F 220/22; G03F 7/00; G03F 7/039; G03F 7/20

(52) U.S. Cl. CPC G03F 7/0045; C07C 309/42; C07C 309/49; C07C 323/64; C07D 333/76; C08F 220/1806; C08F 220/22; G03F 7/0397; C08F 2800/10; G03F 7/2004; G03F 7/70033

(57) ABSTRACT

An onium salt monomer represented by formula (A), where “m40” represents 0 or 1, “m5” represents 2 to 5, “m6” represents 0 to 4, R^{A} represents a hydrogen atom or a methyl group, R² represents a halogen atom other than an iodine atom, a nitro group, a hydroxy group, a cyano group, a hydrocarbyl group, etc., provided that not both of two carbon atoms adjacent to carbon atom having a substituent which is sulfonic acid “SO₃⁻” are bonded to iodine atoms, L^{A}, L^{B}, and L^{C} are each a single bond, an alkylene group, etc., X^{L1} represents a single bond or a hydrocarbylene group, W represents a single bond or a hydrocarbylene group, and Z⁺ represents a sulfonium or iodonium cation. This can provide an onium salt monomer that can realize, when used in a chemically-amplified resist composition, excellent lithography performance and also resistance to pattern collapse even in fine patterning.

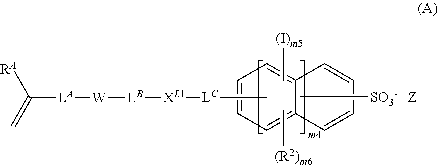

### TECHNICAL FIELD

The present invention relates to: an onium salt monomer; a monomeric photo-acid generator; a polymer; a chemically-amplified resist composition; and a patterning process.

### BACKGROUND ART

As LSIs advance toward higher integration and higher processing speed, miniaturization of pattern rule is progressing rapidly. In particular, the wide-spreading flash memory market and increased storage capacities drive forward the miniaturization. As the advanced miniaturization technology, manufacturing of 65-nm node devices by the ArF lithography has been implemented in a mass scale, and manufacturing of 45-nm node devices by the next-generation ArF immersion lithography is approaching high-volume application. The candidates for the next-generation 32-nm node devices include ultra-high NA lens immersion lithography using a liquid having a higher refractive index than water in combination with a high refractive index lens and a high refractive index resist film, extreme ultraviolet ray (EUV) lithography of wavelength 13.5 nm, and double exposure (double patterning lithography) version of the ArF lithography, on which active research efforts have been made.

As the patterns are miniaturized, the line width roughness (LWR) of line patterns and the critical dimension uniformity (CDU) of hole patterns and dot patterns are regarded significant. In addition, it is pointed out that these factors are affected by the segregation or agglomeration of a base polymer and acid generator and the diffusion of acid. Furthermore, there is a tendency that as the resist film becomes thinner, LWR and CDU become greater, and film thickness reduction to comply with the progress of miniaturization causes degradation of LWR and CDU, which is a serious problem.

Resist compositions for EUV lithography need to meet high sensitivity, high resolution, and low LWR at the same time. As the acid diffusion distance is reduced, LWR is reduced, but sensitivity becomes lower. For example, when the post-exposure baking (PEB) temperature is lowered, the outcome is a reduced LWR, but a lower sensitivity. When the amount of quencher added is increased, the outcome is a reduced LWR, but a lower sensitivity. It is necessary to overcome the tradeoff relation between sensitivity and LWR.

To suppress acid diffusion, proposed is a resist compound containing a repeating unit derived from an onium salt of a sulfonic acid having a polymerizable unsaturated bond (Patent Document 1). The so-called polymer-bound acid generator is capable of generating a polymer-type sulfonic acid upon exposure and therefore, is characterized by a very short distance of acid diffusion. Meanwhile, sensitivity may be enhanced by increasing the proportion of the acid generator. In the case of addition-type acid generators, as the amount added is increased, a higher sensitivity is achievable, but the acid diffusion distance is also increased. Since the acid diffusion is non-uniform, increased acid diffusion leads to degraded LWR and CDU. With respect to a balance among sensitivity, LWR, and CDU, the polymer-type acid generator has a high capability.

Iodine atoms very greatly absorb EUV having a wavelength of 13.5 nm, and therefore, an effect that secondary electrons are generated from iodine atoms during exposure is observed, and iodine atoms are attracting attention in EUV lithography. Patent Document 2 discloses a photo-acid generator having an iodine atom introduced into the anion, and Patent Document 3 discloses a polymerizable-group-containing photo-acid generator having an iodine atom introduced into the anion.

Meanwhile, the effect of perfluoroalkyl compounds (PFAS) on health has been pointed out, and there is a movement in the European REACH to restrict the production and sale of PFAS compounds. Many compounds containing PFAS are currently used in relation with semiconductor lithography. For example, materials containing such compounds are used in surfactants, acid generators, etc. The above-described photo-acid generators disclosed in Patent Documents 1, 2, and 3 are all based on alkanesulfonic acid units, and it is pointed out that these photo-acid generators greatly affect health and the environment.

Patent Document 4 discloses an onium salt containing fluorobenzenesulfonic acid as an alternative to PFAS. In addition, Patent Document 5 discloses an onium salt containing a benzenesulfonic acid. On the other hand, in such benzenesulfonic acid units, the acidity of the sulfonic acid is low, and improvement is required.

### CITATION LIST

### Patent Literature

Patent Document 1: JP4425776B2
Patent Document 2: JP6720926B2
Patent Document 3: JP6973274B2
Patent Document 4: JP2018-159744A
Patent Document 5: JP7531671B2
Patent Document 6: JP7032549B2

### SUMMARY OF INVENTION

### Technical Problem

In the field of acid-catalyzed chemically-amplified resist compositions, there are demands for the development of a resist composition that has higher sensitivity, also allows the improvement of the LWR of line patterns and the CDU of hole patterns, and is also excellent in resolution.

The present invention has been made in view of the above-described circumstances. An object of the present invention is to provide: an onium salt monomer that can realize, when used in a chemically-amplified resist composition, high sensitivity, excellent lithography performance, such as exposure latitude (EL), LWR, CDU, and depth of focus (DOF), and also resistance to pattern collapse even in fine patterning in photolithography using a high-energy beam; a polymer including a repeating unit derived from the onium salt monomer; a chemically-amplified resist composition containing the polymer; and a patterning process using the chemically-amplified resist composition.

### Solution to Problem

To achieve the object, the present invention provides an onium salt monomer represented by the following formula (A),

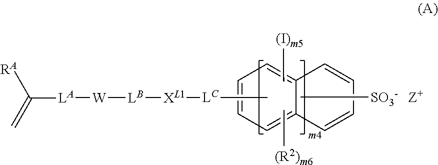

wherein “m4” represents an integer of 0 or 1, “m5” represents an integer of 2 or 3 when “m4” is 0 and represents an integer of 2, 3, 4, or 5 when “m4” is 1, and “m6” represents an integer of 0, 1, or 2 when “m4” is 0 and represents an integer of 0, 1, 2, 3, or 4 when “m4” is 1, provided that when “m4” is 0, 2≤m5+m6≤4 and when “m4” is 1, 2≤m5+m6≤6;
R^{A} represents a hydrogen atom or a methyl group;
R² represents a halogen atom other than an iodine atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m6” is 2 or more, the R²s being identical to or different from each other and the R²s optionally being bonded to each other to form a ring together with the carbon atoms bonded thereto, provided that not both of the two carbon atoms adjacent to the carbon atom having a substituent which is the sulfonic acid “SO₃⁻” are bonded to iodine atoms;
L^{A}, L^{B}, and L^{C} each independently represent a single bond, an ester group, an ether group, an amide group, a sulfonic acid ester group, or a linear, branched, or cyclic alkylene group having 1 to 12 carbon atoms, the alkylene group optionally containing one or more selected from an ester group, an ether group, an amide group, and a sulfonic acid ester group;
X^{L1} represents a single bond or a hydrocarbylene group having 1 to 40 carbon atoms and optionally containing a heteroatom;
W represents a single bond or a hydrocarbylene group having 1 to 30 carbon atoms, the hydrocarbylene group optionally having at least one selected from an oxygen atom, a nitrogen atom, a sulfur atom, and a halogen atom;
Z⁺ represents a sulfonium cation represented by the following formula (Z-1) or an iodonium cation represented by the following formula (Z-2),

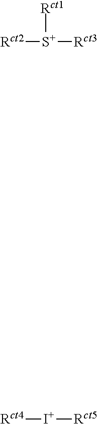

wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.

Particularly in photolithography using a high-energy beam, such as a KrF excimer laser beam, an ArF excimer laser beam, an electron beam (EB), or EUV, such an onium salt monomer has high sensitivity and high contrast, and can be used for a chemically-amplified resist composition that is excellent in lithography performance, such as exposure latitude (EL), LWR, CDU, and depth of focus (DOF), and is also resistant to pattern collapse even in fine patterning.

The inventive onium salt monomer is preferably one represented by the following formula (A1),

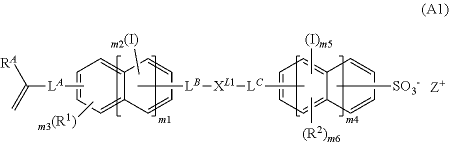

wherein “m1” represents an integer of 0 or 1, “m2” represents an integer of 0, 1, 2, or 3 when “m1” is 0 and represents an integer of 0, 1, 2, 3, 4, or 5 when “m1” is 1, and “m3” represents an integer of 0, 1, 2, 3, or 4 when “m1” is 0 and represents an integer of 0, 1, 2, 3, 4, 5, or 6 when “m1” is 1, provided that when “m1” is 0, 0≤m2+m3≤4 and when “m1” is 1, 0≤m2+m3≤6;
R¹ represents a halogen atom other than an iodine atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m3” is 2 or more, the R's being identical to or different from each other and the R's optionally being bonded to each other to form a ring together with the carbon atoms bonded thereto; and “m4” to “m6”, R^{A}, R², X^{L1}, L^{A}, L^{B}, L^{C}, and Z⁺ are as defined above.

In this case, the inventive onium salt monomer is more preferably one represented by the following formula (A2),

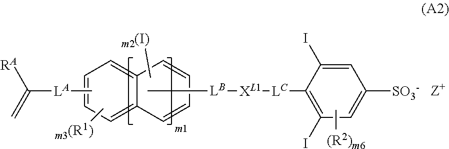

wherein “m1” to “m3”, “m6”, R^{A}, R¹, R², X^{L1}, L^{A}, L^{B}L^{C}, and Z⁺ are as defined above.

When the inventive onium salt monomer has such a structure, the advantageous effects of the present invention can be exhibited more fully.

The present invention also provides a monomeric photo-acid generator comprising the above-described onium salt monomer.

The inventive onium salt monomer can be used suitably as a monomeric photo-acid generator.

The present invention also provides a polymer comprising a repeating unit derived from the above-described monomeric photo-acid generator.

The inventive onium salt monomer can be used particularly suitably as a polymer-bound photo-acid generator.

The polymer preferably further comprises a repeating unit represented by the following formula (b1) or (b2),

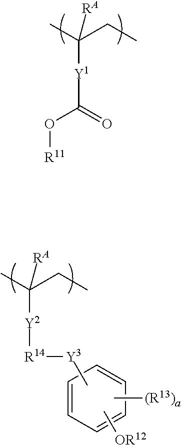

wherein each R^{A} independently represents a hydrogen atom or a methyl group;
Y¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally having at least one selected from a hydroxy group, a halogen atom, a saturated hydrocarbyloxy group having 1 to 8 carbon atoms, and a saturated hydrocarbylcarbonyloxy group having 2 to 8 carbon atoms;
Y² represents a single bond or an ester bond;
Y³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.

Such a polymer can be used suitably for a chemically-amplified resist composition.

The present invention also provides a chemically-amplified resist composition comprising the above-described polymer as a base polymer.

Particularly in photolithography using a high-energy beam, such as a KrF excimer laser beam, an ArF excimer laser beam, an electron beam (EB), or EUV, such a chemically-amplified resist composition has high sensitivity and high contrast, is excellent in lithography performance, such as exposure latitude (EL), LWR, CDU, and depth of focus (DOF), and is also resistant to pattern collapse even in fine patterning.

The inventive chemically-amplified resist composition preferably further comprises at least one kind selected from an organic solvent, a quencher, an acid generator, a surfactant, and a dissolution inhibitor.

The inventive chemically-amplified resist composition may contain these components as necessary.

The present invention also provides a patterning process comprising the steps of:
forming a resist film on a substrate by using the above-described chemically-amplified resist composition;
exposing the resist film by using a high-energy beam; and
developing the exposed resist film by using a developer.

In photolithography using a high-energy beam, the inventive patterning process can achieve high sensitivity and high contrast, excellent lithography performance, such as exposure latitude (EL), LWR, CDU, and depth of focus (DOF), and also resistance to pattern collapse even in fine patterning.

It is preferable that the high-energy beam is an ArF excimer laser beam having a wavelength of 193 nm, a KrF excimer laser beam having a wavelength of 248 nm, an electron beam, or an extreme ultraviolet ray having a wavelength of 3 to 15 nm.

When such high-energy beams are used, the inventive patterning process can exhibit the advantageous effects of the present invention more fully.

### Advantageous Effects of Invention

In a polymer that contains a benzenesulfonic acid having multiple substituents which are iodine atoms and that includes a repeating unit that generates an acid upon exposure, secondary electrons are generated from the iodine atoms during exposure in EUV lithography of wavelength 13.5 nm, since iodine atoms very greatly absorb EUV. By virtue of the resonance effect of the aromatic group, a stronger acid can be achieved when substituents which are iodine atoms are contained compared to an arylsulfonic acid only containing substituents which are fluorine atoms, and also by virtue of the high EUV absorption property of iodine atoms, acidity is enhanced. Furthermore, the mobility of the sulfonic acid is reduced by iodine atoms, having a great atomic weight, being present nearby, and acid diffusion can be suppressed and it is possible to improve LWR and CDU. There is a characteristic that out of the two carbon atoms present in the ortho positions of the sulfonic acid, one is not bonded to an iodine atom, and therefore, the sulfonic acid is not affected by the sterically bulky electron clouds of iodine atoms, and favorable sensitivity and favorable resolution can be exhibited.

### DESCRIPTION OF EMBODIMENTS

As described above, there have been demands for the development of: an onium salt monomer that can realize, when used in a chemically-amplified resist composition, high sensitivity, excellent lithography performance, such as exposure latitude (EL), LWR, CDU, and depth of focus (DOF), and also resistance to pattern collapse even in fine patterning in photolithography using a high-energy beam; a polymer including a repeating unit derived from the onium salt monomer; a chemically-amplified resist composition containing the polymer; and a patterning process using the chemically-amplified resist composition.

Meanwhile, Patent Document 6 shows a structure in which the ortho positions of a benzenesulfonic acid are each substituted with an aryl group. It is possible to use such a structure as an alternative to PFAS, but it can be assumed that sensitivity and resolution are not sufficient due to the presence of sterically bulky groups near the sulfonate anion.

To achieve the object, the present inventors have studied earnestly and found out that, by using, as a polymer-bound photo-acid generator, a polymer including a repeating unit derived from an onium salt monomer which contains a benzenesulfonic acid having multiple substituents which are iodine atoms, one of the two carbon atoms in the ortho positions of the sulfonic acid not being bonded to an iodine atom, it is possible to obtain a chemically-amplified resist composition having high sensitivity, high contrast, high resolution, and improved lithography performance such as LWR and CDU. Thus, the present invention has been completed.

That is, the present invention is
an onium salt monomer represented by the following formula (A),

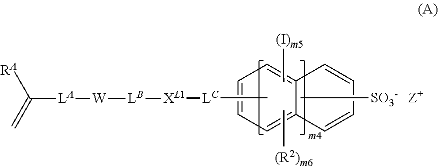

wherein “m4” represents an integer of 0 or 1, “m5” represents an integer of 2 or 3 when “m4” is 0 and represents an integer of 2, 3, 4, or 5 when “m4” is 1, and “m6” represents an integer of 0, 1, or 2 when “m4” is 0 and represents an integer of 0, 1, 2, 3, or 4 when “m4” is 1, provided that when “m4” is 0, 2≤m5+m6≤4 and when “m4” is 1, 2≤m5+m6≤6;
R^{A} represents a hydrogen atom or a methyl group;
R² represents a halogen atom other than an iodine atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m6” is 2 or more, the R²s being identical to or different from each other and the R²s optionally being bonded to each other to form a ring together with the carbon atoms bonded thereto, provided that not both of the two carbon atoms adjacent to the carbon atom having a substituent which is the sulfonic acid “SO₃⁻” are bonded to iodine atoms;
L^{A}, L^{B}, and L^{C} each independently represent a single bond, an ester group, an ether group, an amide group, a sulfonic acid ester group, or a linear, branched, or cyclic alkylene group having 1 to 12 carbon atoms, the alkylene group optionally containing one or more selected from an ester group, an ether group, an amide group, and a sulfonic acid ester group;
X^{L1} represents a single bond or a hydrocarbylene group having 1 to 40 carbon atoms and optionally containing a heteroatom;
W represents a single bond or a hydrocarbylene group having 1 to 30 carbon atoms, the hydrocarbylene group optionally having at least one selected from an oxygen atom, a nitrogen atom, a sulfur atom, and a halogen atom;
Z⁺ represents a sulfonium cation represented by the following formula (Z-1) or an iodonium cation represented by the following formula (Z-2),

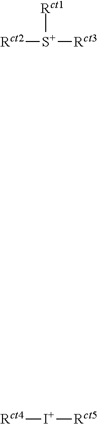

wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.

Hereinafter, the present invention will be described in detail, but the present invention is not limited thereto.

### [Onium Salt Monomer]

The inventive onium salt monomer is represented by the following formula (A).

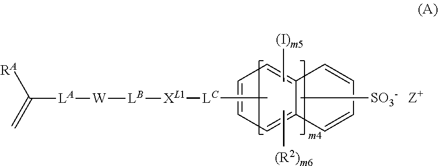

In the formula (A), “m4” represents an integer of 0 or 1. When “m4” is 0, a benzene ring is indicated, and when “m4” is 1, a naphthalene ring is indicated, and from the viewpoint of solvent solubility, a benzene ring, where “m4” is 0, is preferable. “m5” represents an integer of 2 or 3 when “m4” is 0 and represents an integer of 2, 3, 4, or 5 when “m4” is 1. From the viewpoint of the procurement of raw materials, “m5” is preferably 2. “m6” represents an integer of 0, 1, or 2 when “m4” is 0 and represents an integer of 0, 1, 2, 3, or 4 when “m4” is 1, provided that when “m4” is 0, 2≤m5+m6≤4 and when “m4” is 1, 2≤m5+m6≤6. By multiple hydrogen atoms on the benzenesulfonic acid being substituted with iodine atoms, absorption of EUV light is enhanced, furthermore, the degree of freedom of the sulfinic acid is reduced by the influence of the volume of the iodine atoms, and thus, a material having high sensitivity and low acid diffusion can be achieved. Meanwhile, at least one ortho position of SO₃⁻ is not substituted with an iodine atom, and it is further preferable that neither of the ortho positions is substituted with an iodine atom. Such an onium salt monomer can reduce the steric hindrance around the sulfonate salt and can generate an acid effectively, and therefore, it is possible to provide a resist composition excellent in sensitivity and resolution.

In the formula (A), W represents a single bond or a hydrocarbylene group having 1 to 30, preferably 1 to 20 carbon atoms, the hydrocarbylene group optionally having at least one selected from an oxygen atom, a nitrogen atom, a sulfur atom, and a halogen atom. Specific examples include ones shown below, but are not limited thereto. Note that, in the following formulae, “*” represents an attachment point to each of L^{A} and L^{B}.

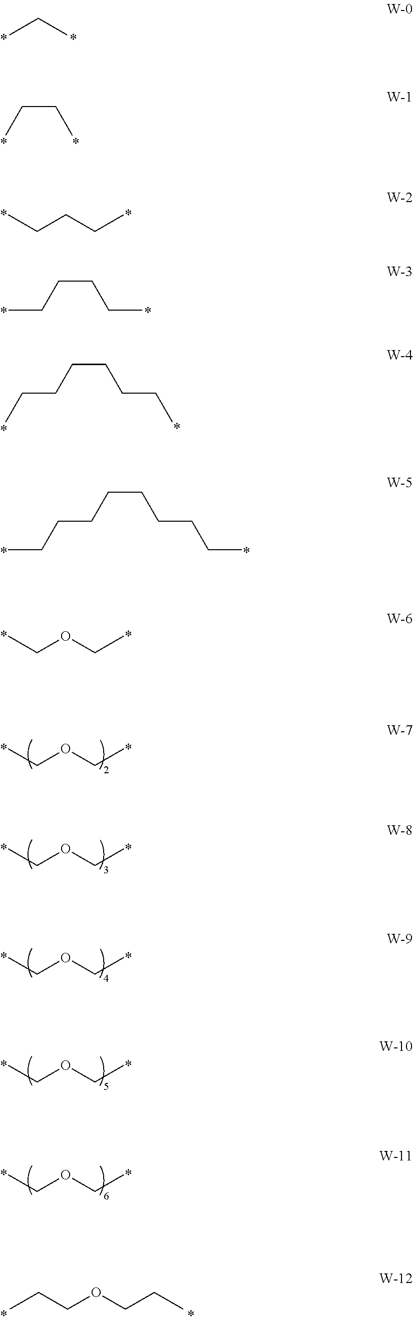

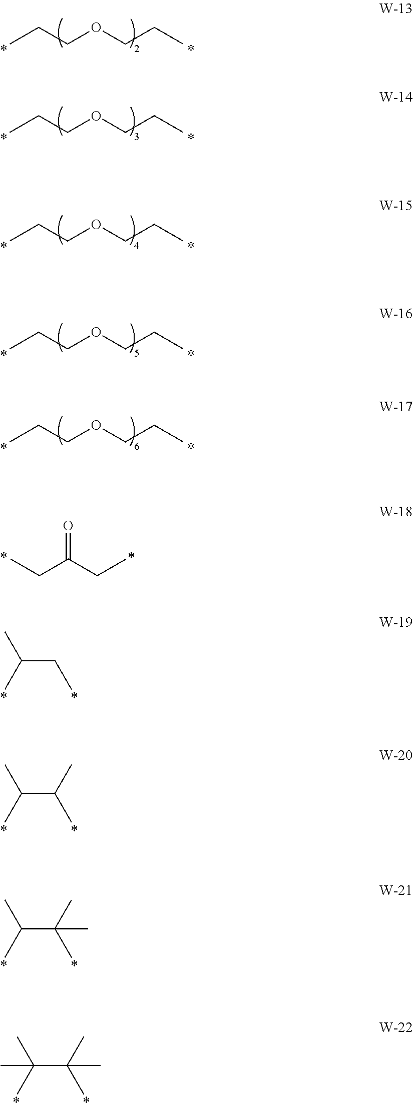

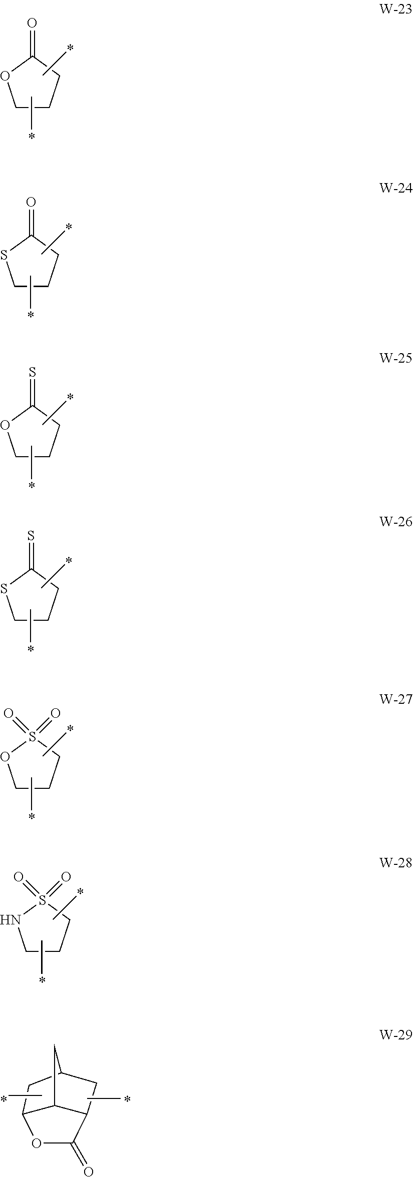

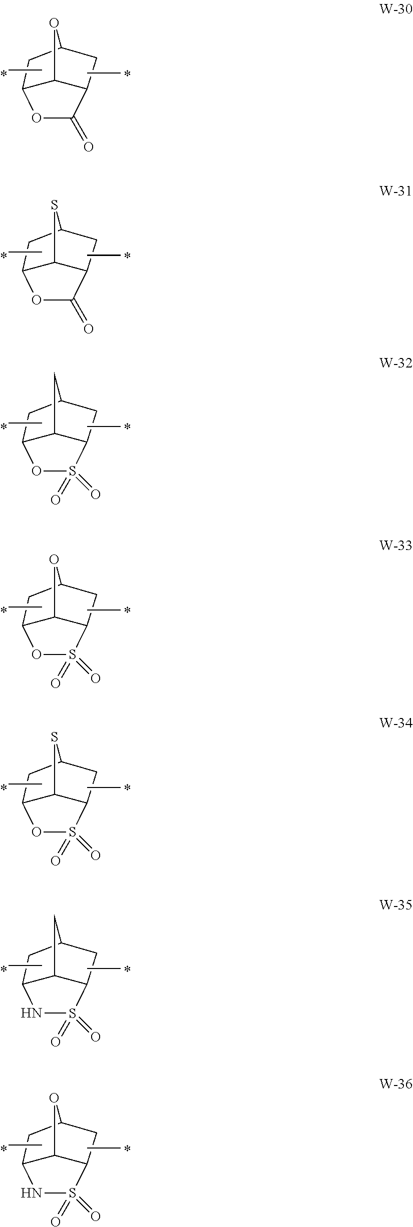

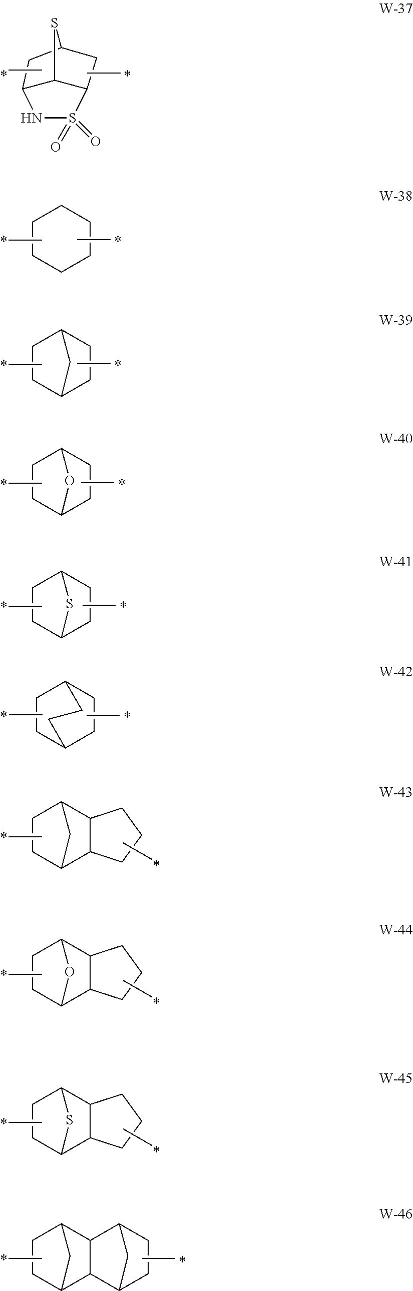

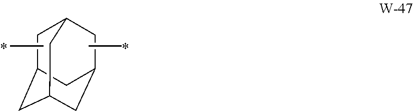

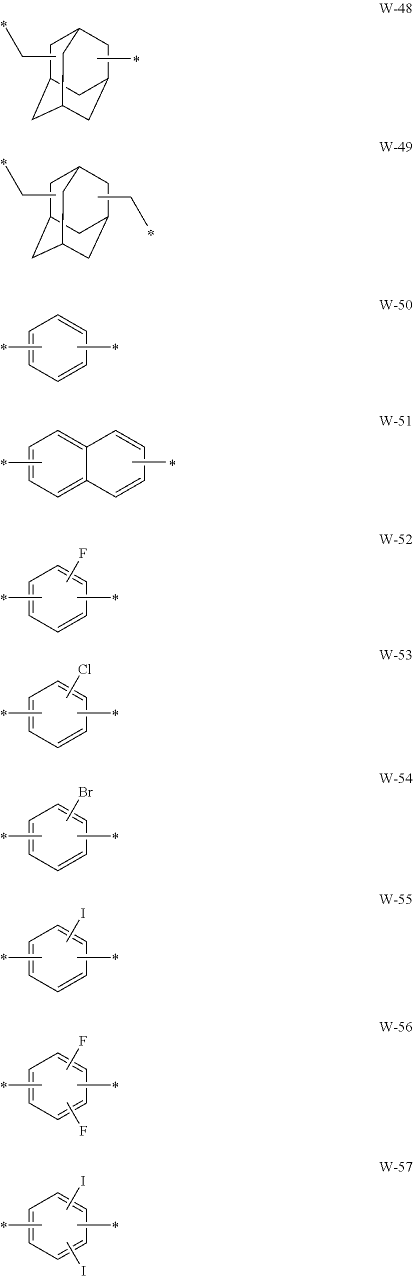

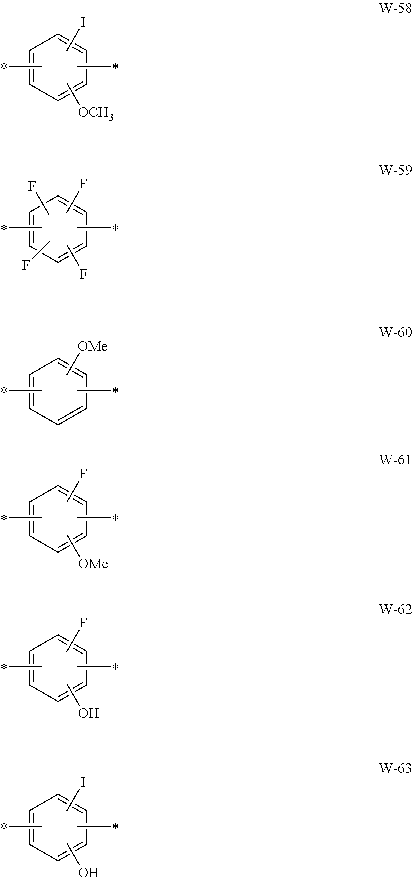

In the formula (A), X^{L1} represents a single bond or a hydrocarbylene group having 1 to 40 carbon atoms and optionally containing a heteroatom. Specific examples thereof include alkanediyl groups, cyclic saturated hydrocarbylene groups, and arylene groups. Examples of the heteroatom include an oxygen atom, a nitrogen atom, and a sulfur atom.

Specific examples of the hydrocarbylene group represented by X^{L1} having 1 to 40 carbon atoms and optionally containing a heteroatom include ones shown below, but are not limited thereto. Note that, in the following formulae, “*” represents an attachment point to each of L^{B} and L^{C}.

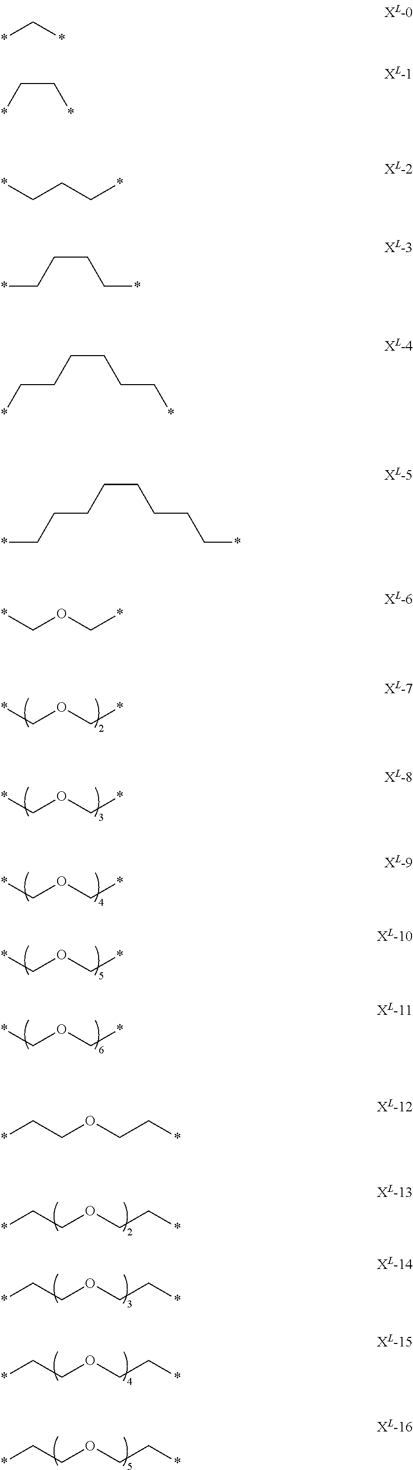

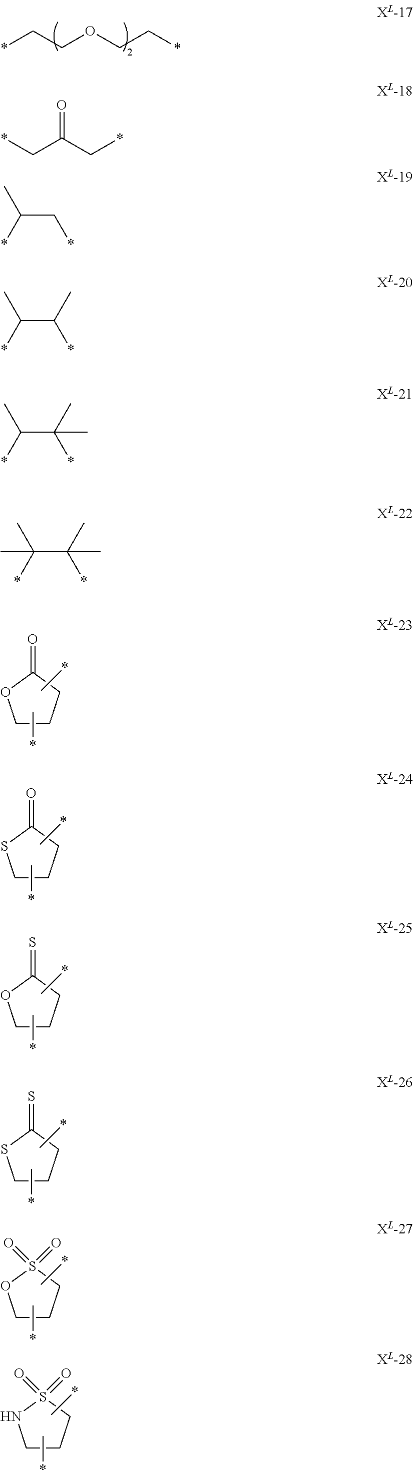

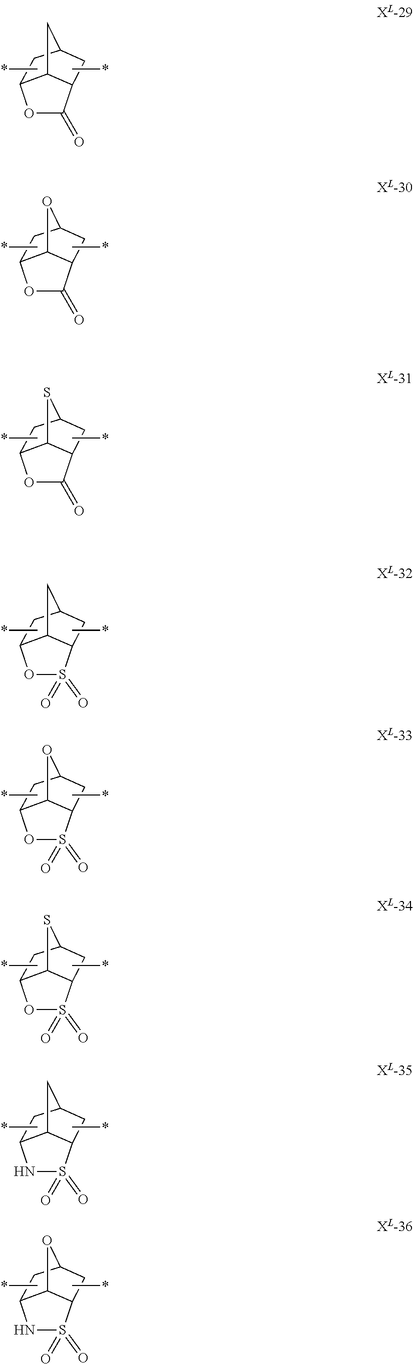

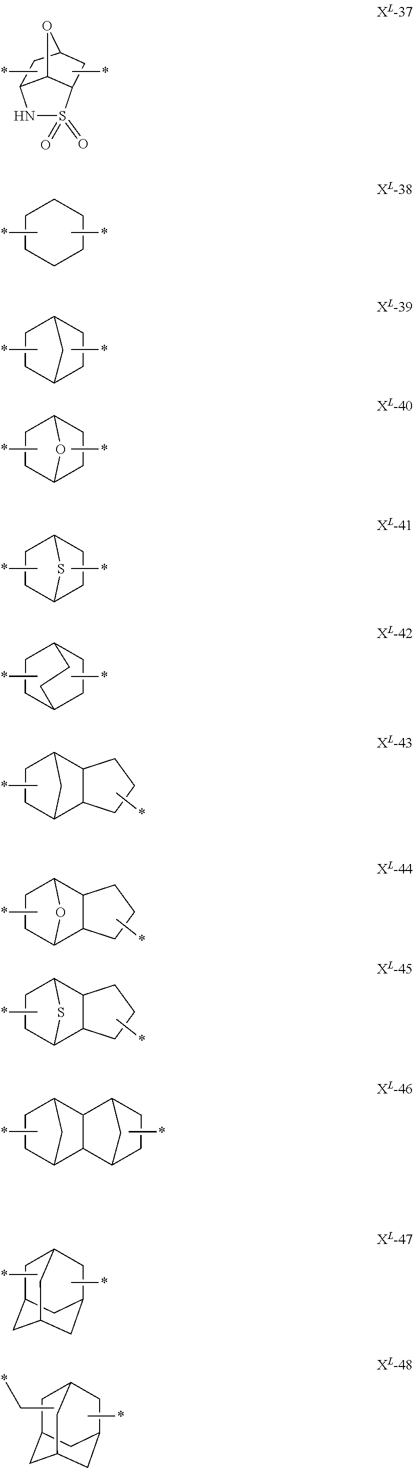

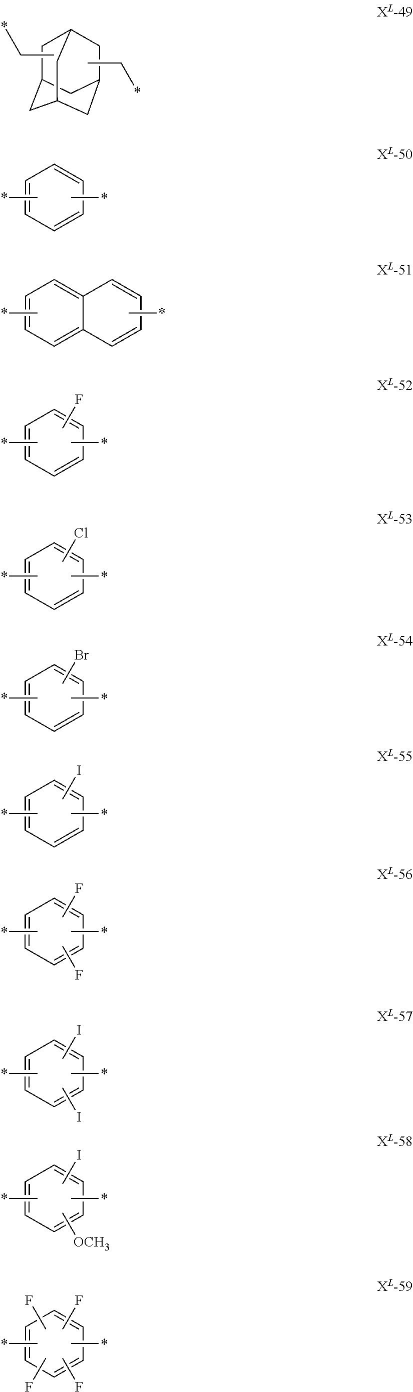

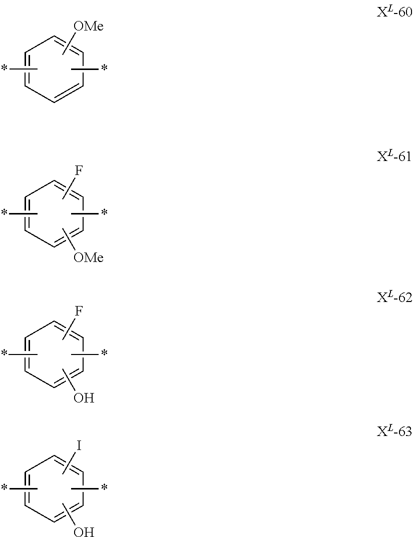

Among these, X^{L}-0 to X^{L}-22, X^{L}-29 to X^{L}-34, and XL-47 to X^{L}-63 are preferable.

R² represents a halogen atom other than an iodine atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m6” is 2 or more, the R²s being identical to or different from each other and the R²s optionally being bonded to each other to form a ring together with the carbon atoms bonded thereto. Here, as the R², preferable are a halogen atom other than an iodine atom, a nitro group, a hydroxy group, a cyano group, and a hydrocarbyl group having 1 to 3 carbon atoms and optionally containing a heteroatom; and more preferable are a fluorine atom, a nitro group, a cyano group, and a hydrocarbyl group having 1 to 3 carbon atoms and optionally containing a heteroatom. In such an onium salt monomer, the surroundings of the sulfonate anion are not sterically crowded, and therefore, such an onium salt monomer is useful for achieving high sensitivity and high resolution.

Furthermore, when “m6” is 2, 3, or 4, multiple R²s may be bonded to each other to form a ring together with the carbon atoms bonded thereto. As the ring, five- to eight-membered rings are preferable.

In the formula (A), L^{A}, L^{B}, and L^{C} each independently represent a single bond, an ester group, an ether group, an amide group, a sulfonic acid ester group, or a linear, branched, or cyclic alkylene group having 1 to 12 carbon atoms, the alkylene group optionally containing one or more selected from an ester group, an ether group, an amide group, and a sulfonic acid ester group. Among these, as L^{A}, a single bond or an ester bond is preferable, and a single bond is more preferable. When L^{A} is a single bond, the mobility around the main chain of the photo-acid generator incorporated in the polymer main chain is reduced, and thus, acid diffusion can be further suppressed. As L^{B}, a single bond, an ether bond, an ester bond, or a sulfonic acid ester bond is preferable, and a single bond or an ester bond is more preferable. By the distance between the polymer main chain and the sulfonic acid being shortened, acid diffusion can be further suppressed. As L^{C}, a single bond, an ether bond, an ester bond, or a sulfonic acid ester bond is preferable, and an ether bond, an ester bond, or a sulfonic acid ester bond is more preferable.

As the onium salt monomer represented by the formula (A), ones represented by the following formula (A1) are preferable.

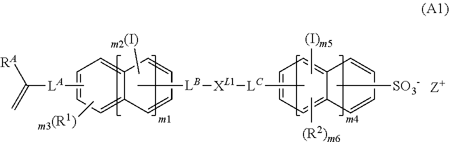

In the formula, “m1” represents an integer of 0 or 1, “m2” represents an integer of 0, 1, 2, or 3 when “m1” is 0 and represents an integer of 0, 1, 2, 3, 4, or 5 when “m1” is 1, and “m3” represents an integer of 0, 1, 2, 3, or 4 when “m1” is 0 and represents an integer of 0, 1, 2, 3, 4, 5, or 6 when “m1” is 1, provided that when “m1” is 0, 0≤m2+m3≤4 and when “m1” is 1, 0≤m2+m3≤6;
R¹ represents a halogen atom other than an iodine atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m3” is 2 or more, the R's being identical to or different from each other and the R's optionally being bonded to each other to form a ring together with the carbon atoms bonded thereto; and
“m4” to “m6”, R^{A}, R², X^{L1}, L^{A}, L^{B}, L^{C}, and Z⁺ are as defined above.

As the onium salt monomers represented by the formulae (A) and (A1), ones represented by the following formula (A2) are preferable.

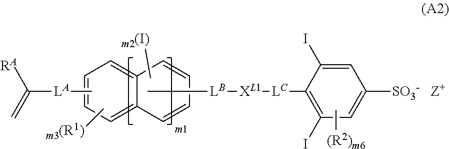

In the formula, “m1” to “m3”, “m6”, R^{A}, R¹, R², X^{L1}, L^{A}, L^{B}, L^{C}, and Z⁺ are as defined above.

Specific examples of the anion of the onium salt represented by the formula (A) include ones shown below, but are not limited thereto. Note that, in the following formulae, R^{A} is as defined above and Me represents a methyl group. Furthermore, the attachment positions of the substituents on the aromatic rings may change places with each other.

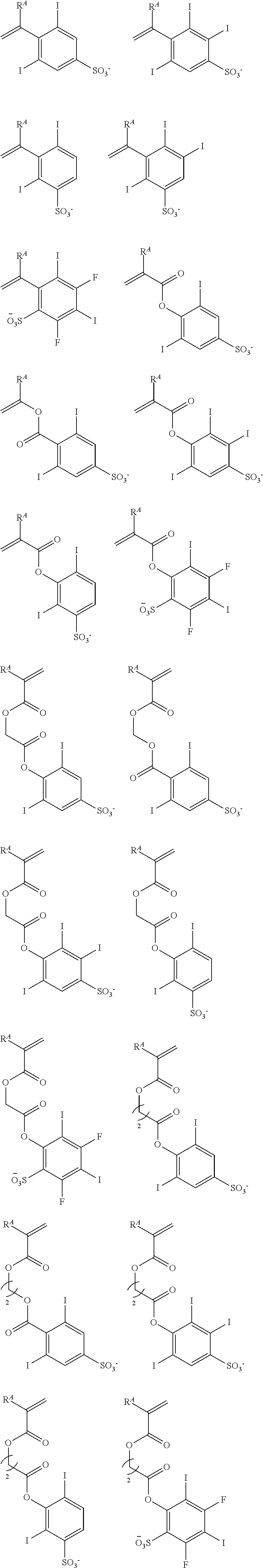

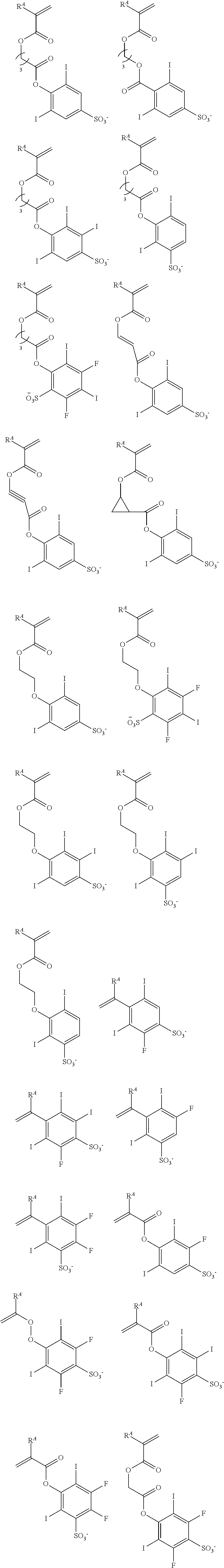

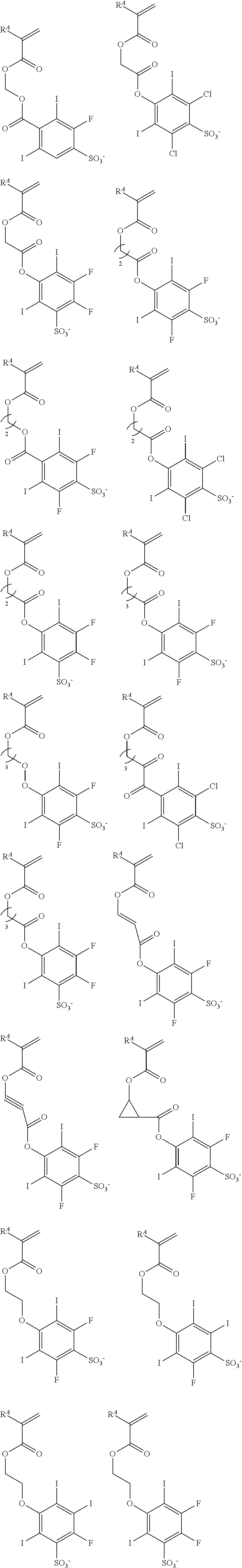

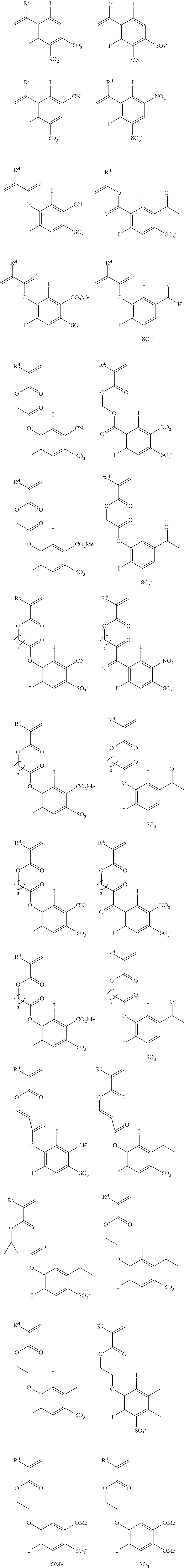

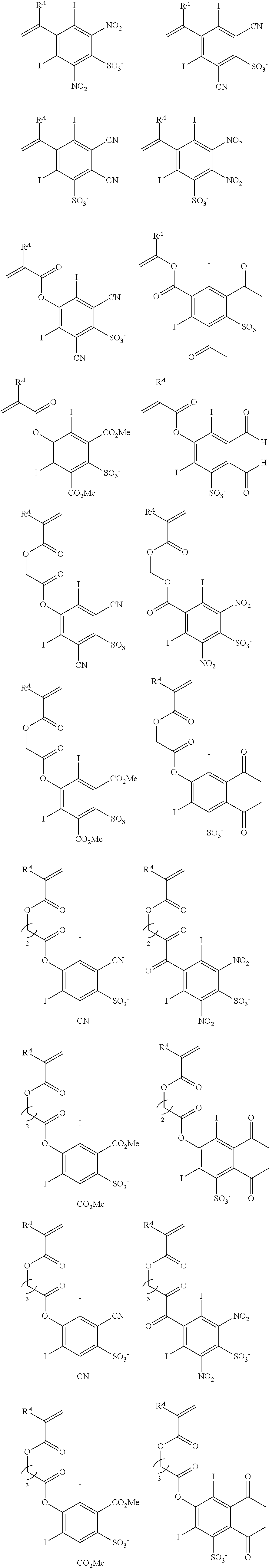

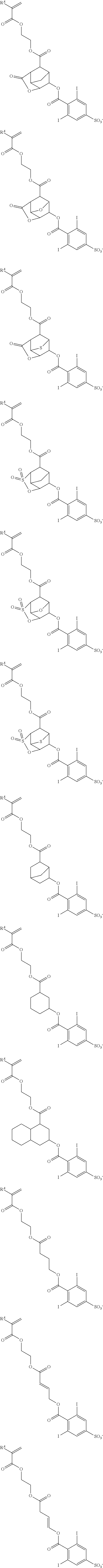

[chemical structure]

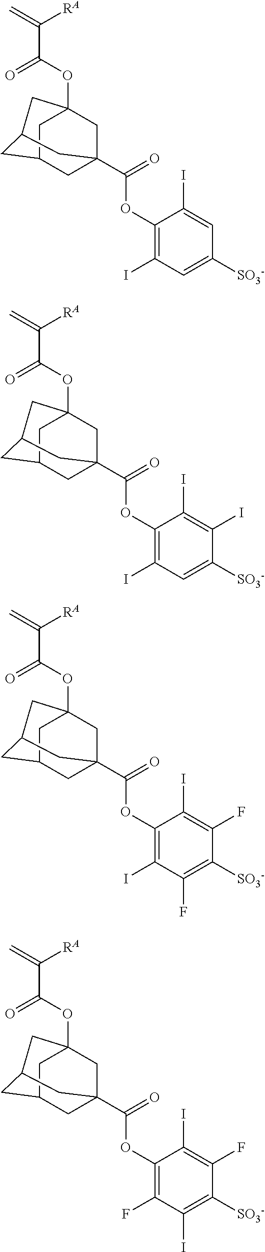

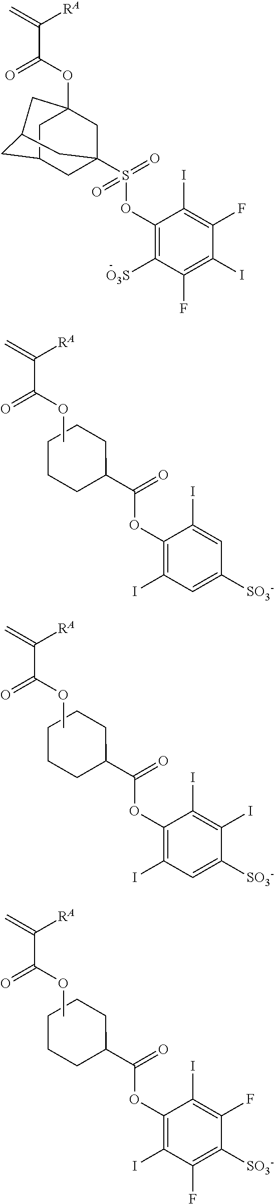

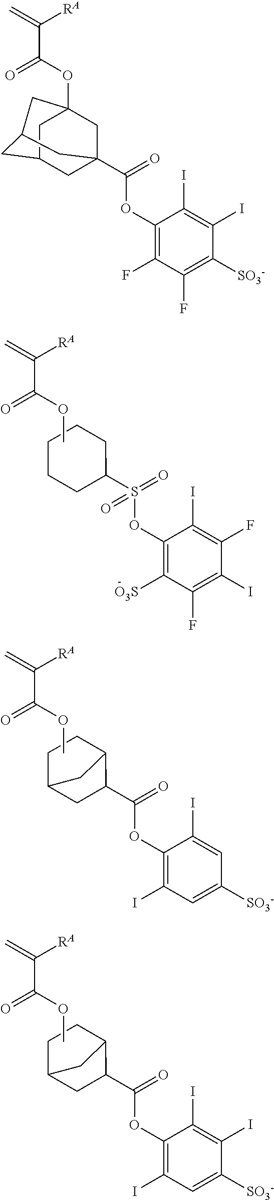

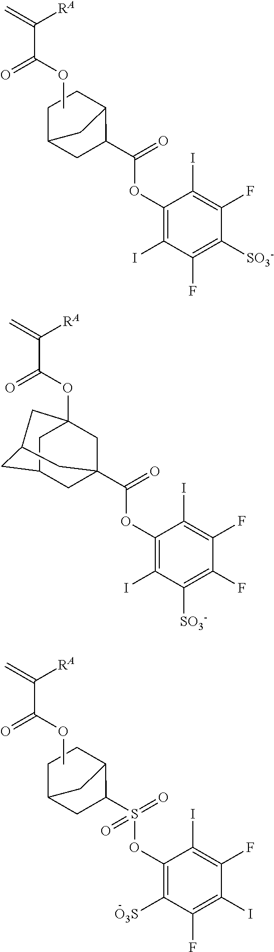

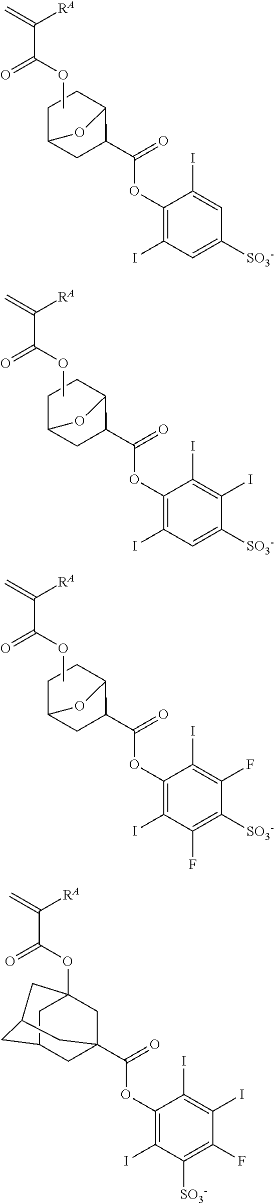

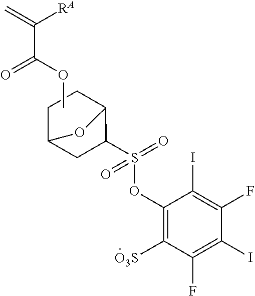

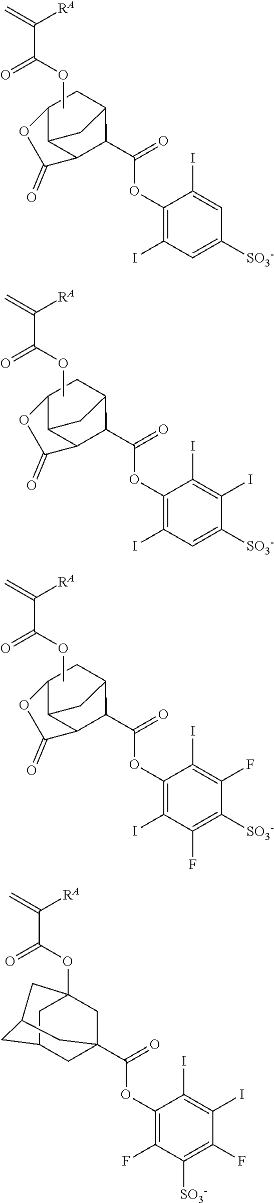

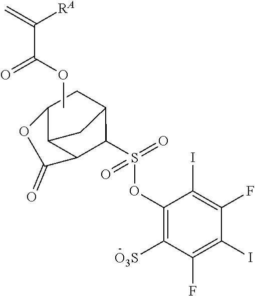

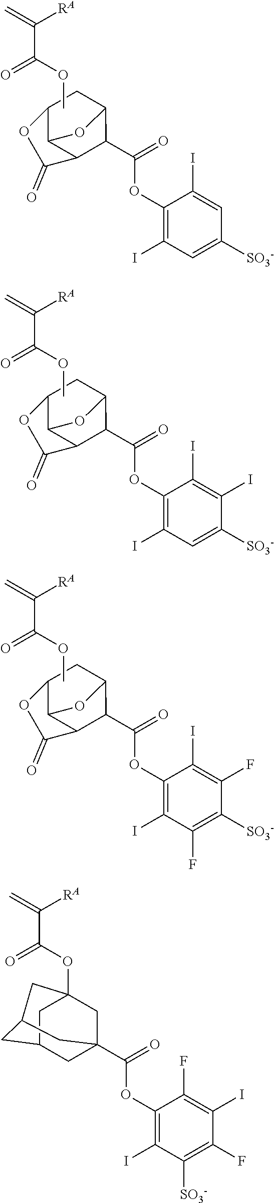

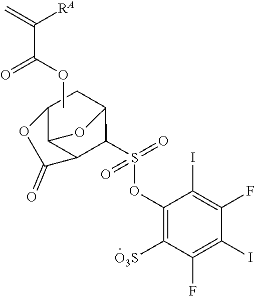

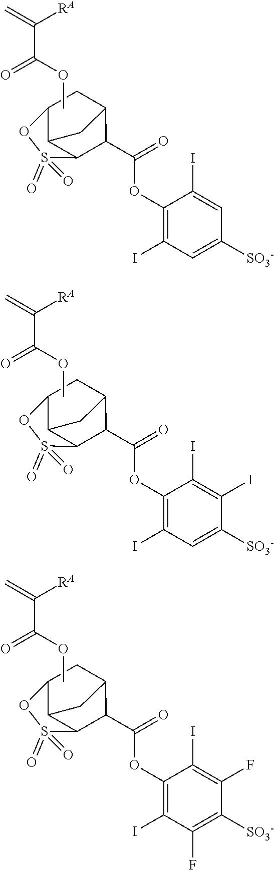

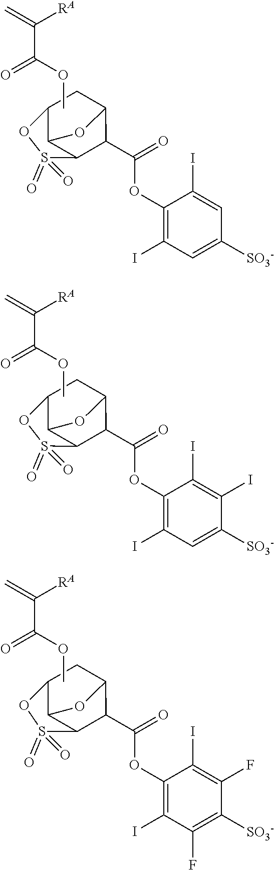

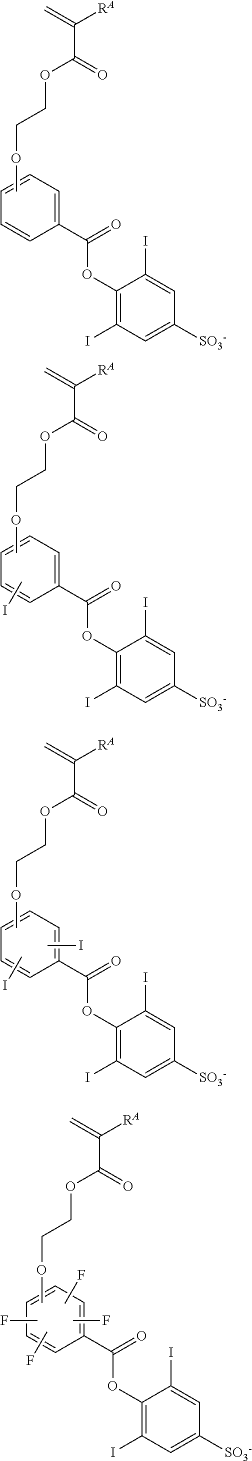

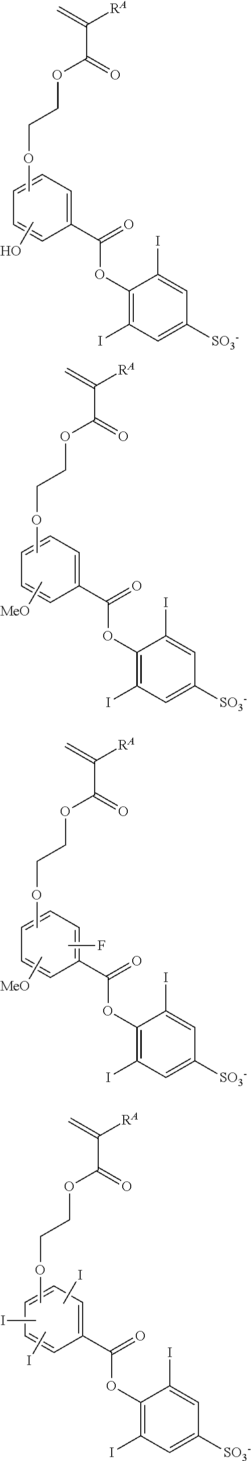

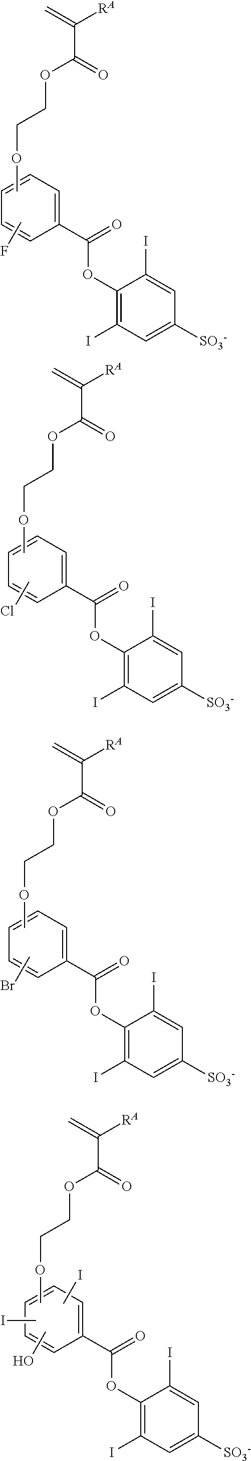

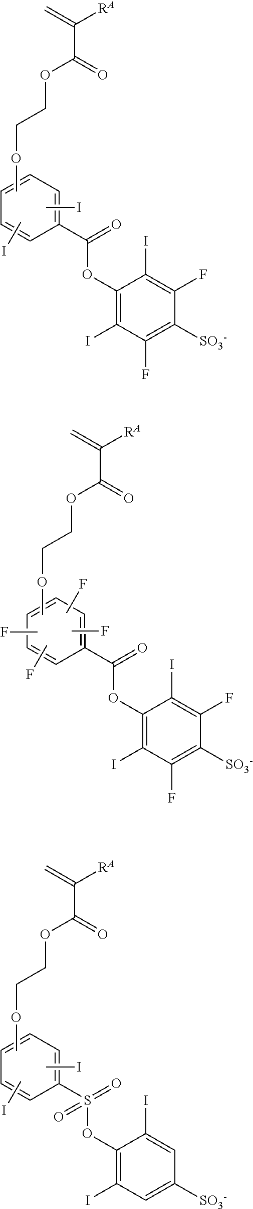

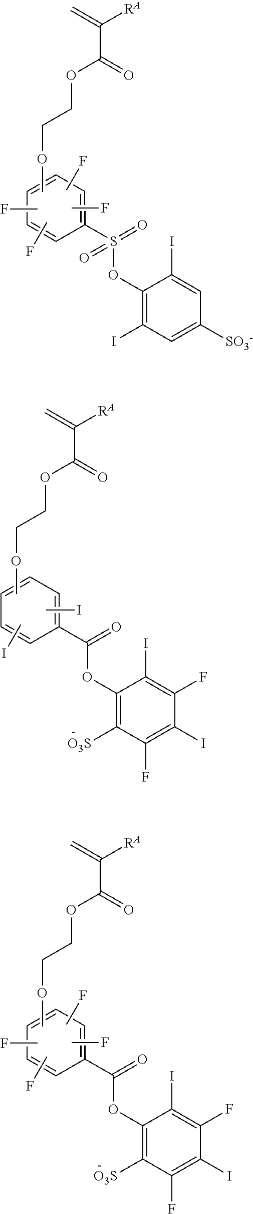

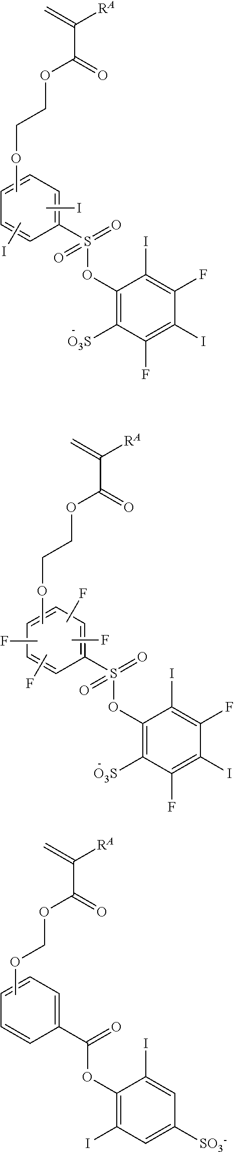

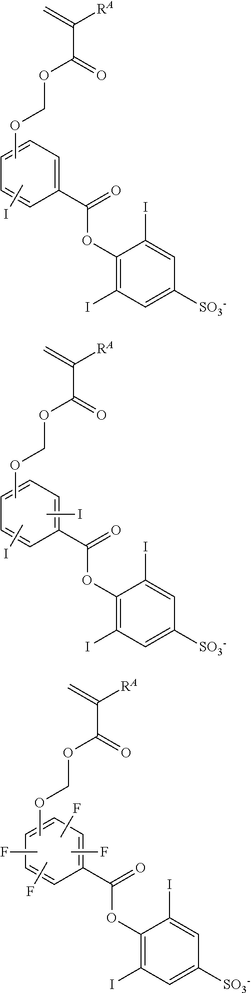

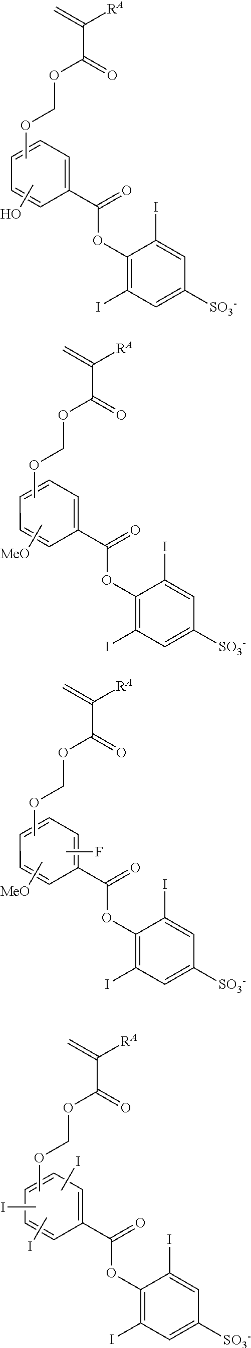

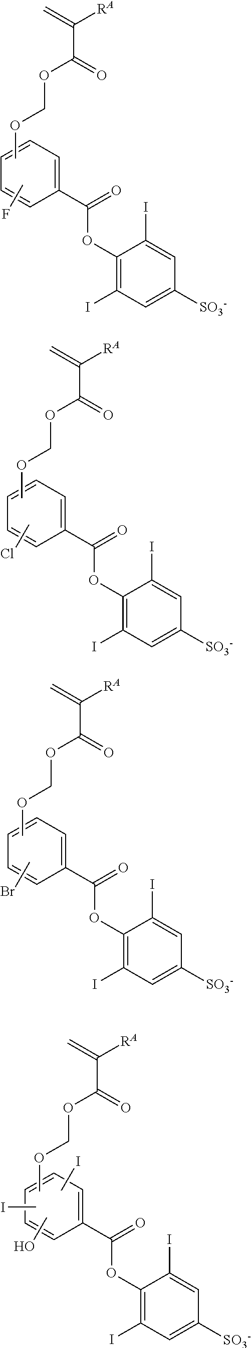

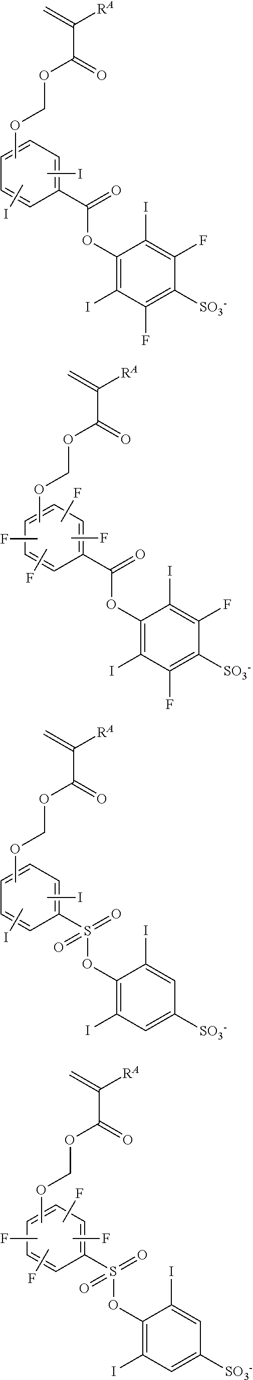

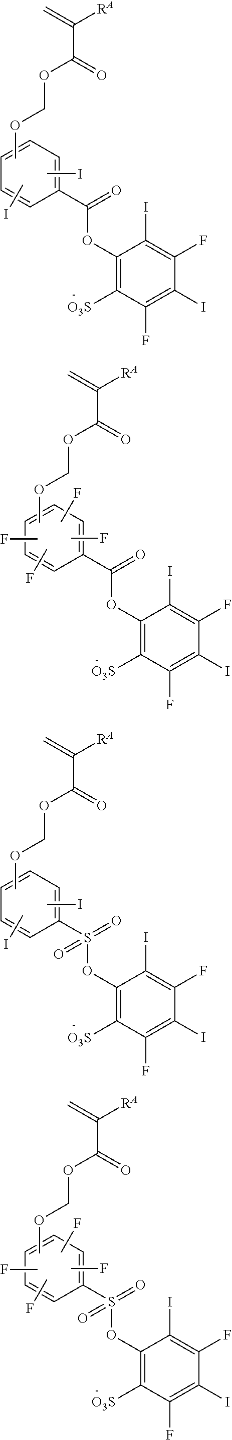

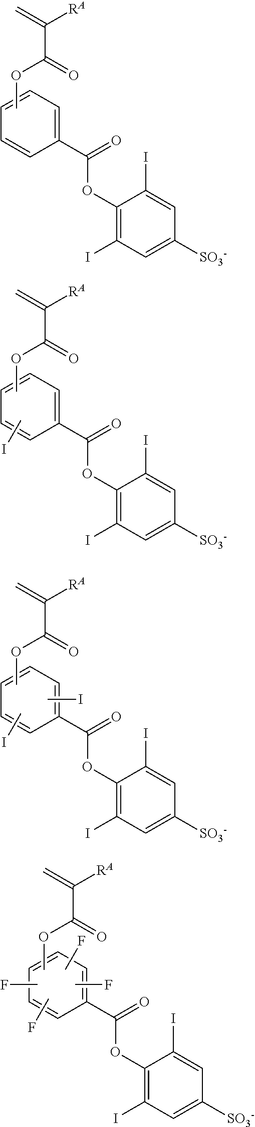

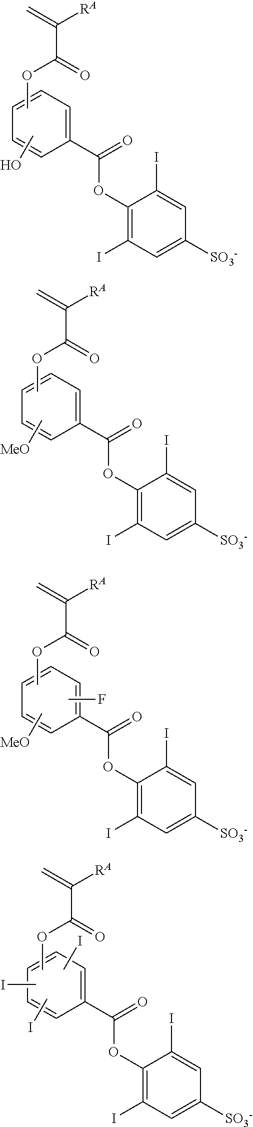

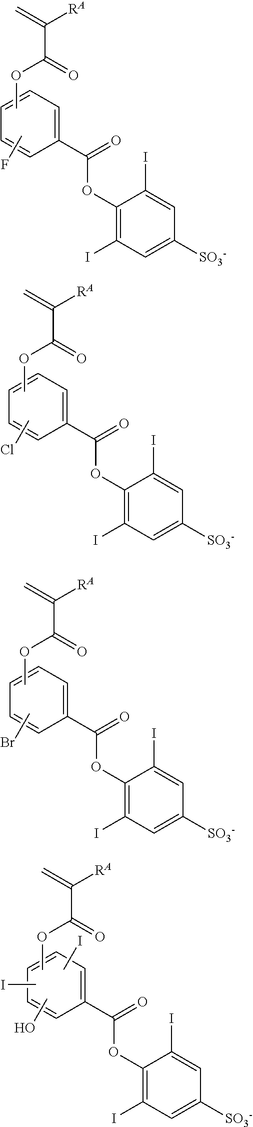

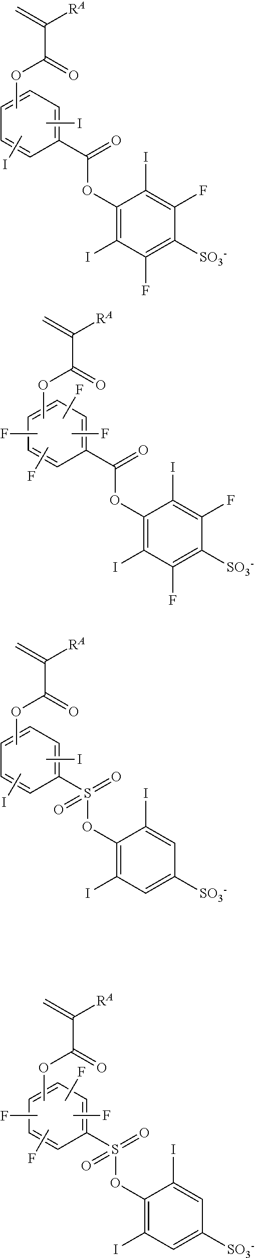

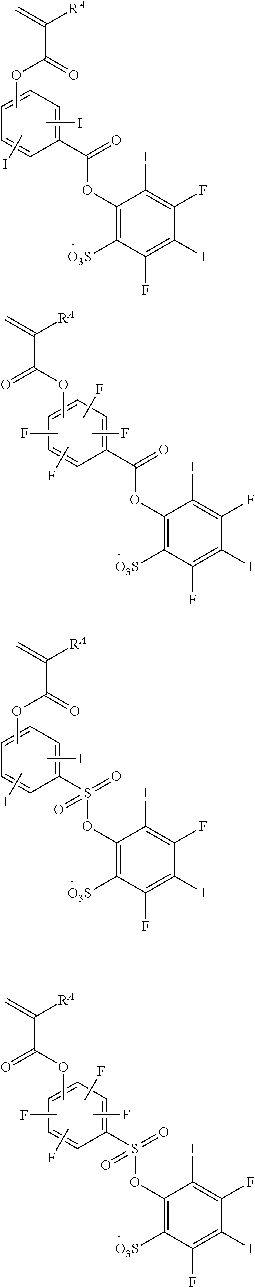

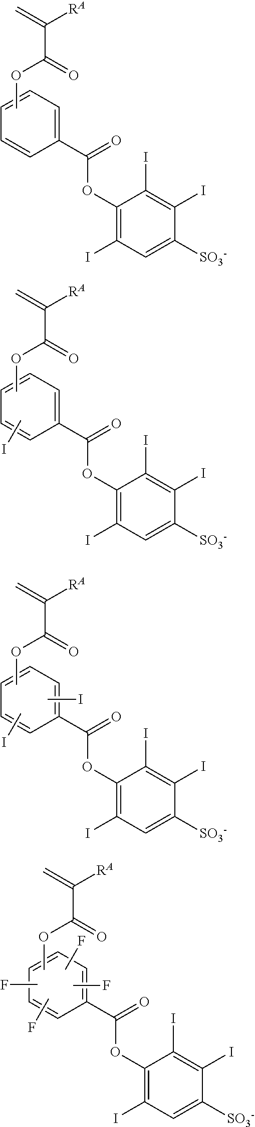

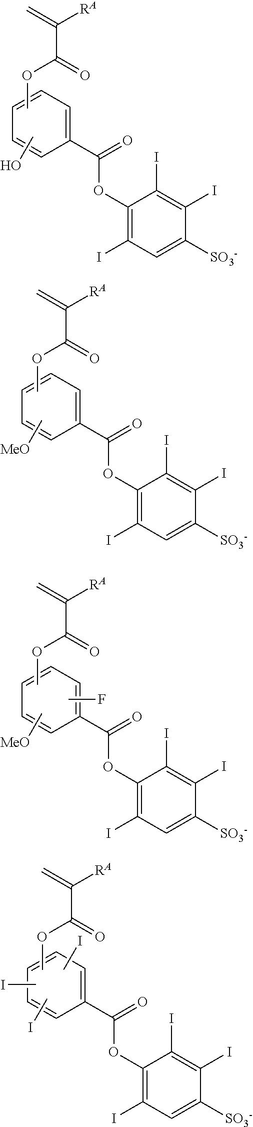

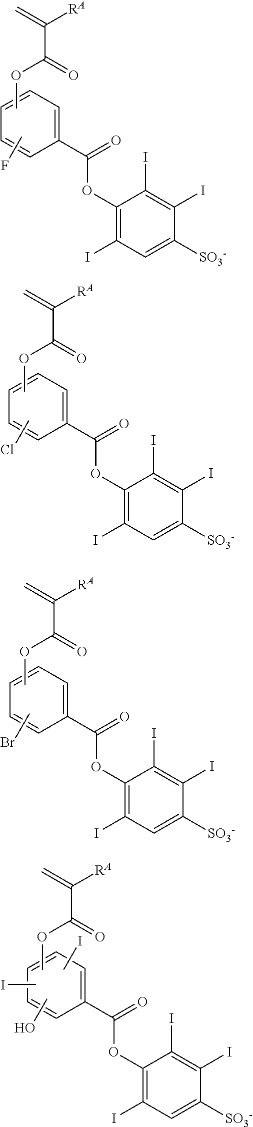

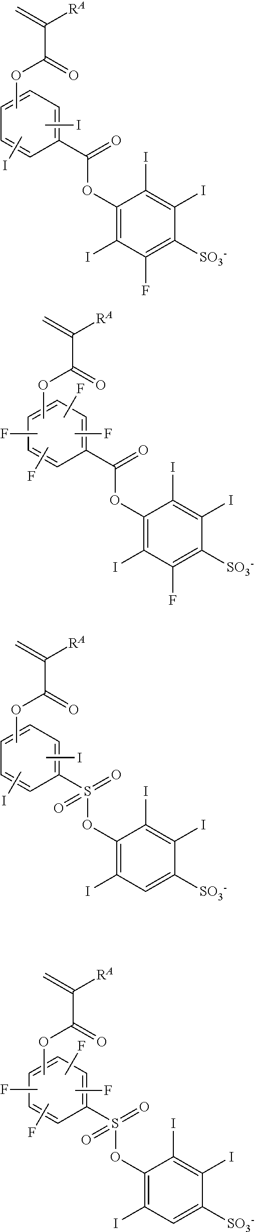

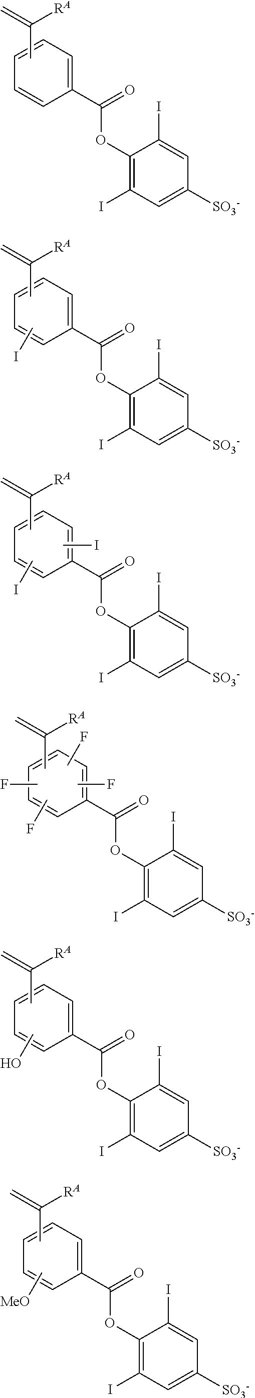

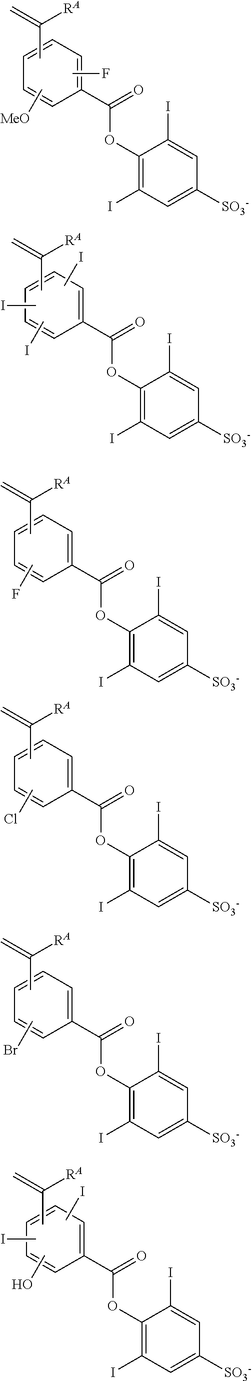

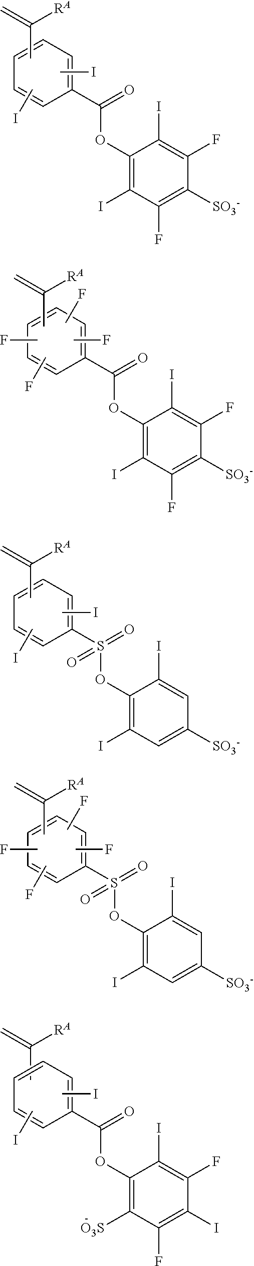

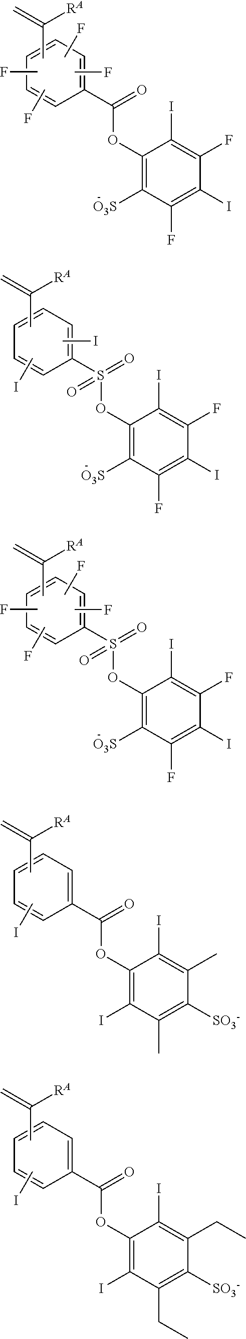

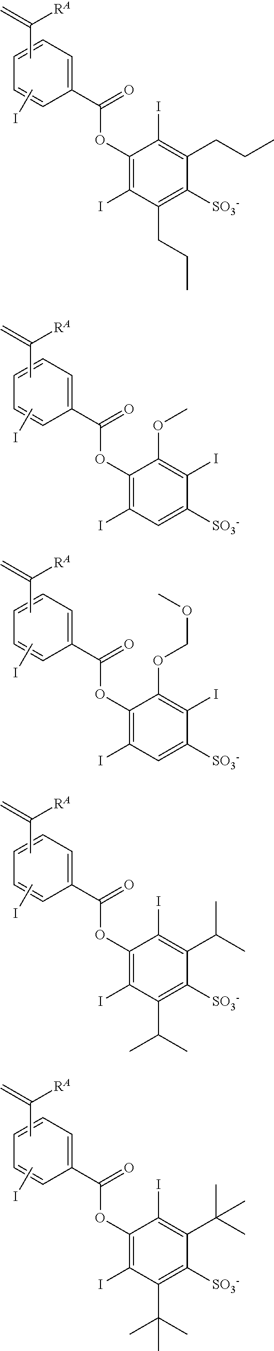

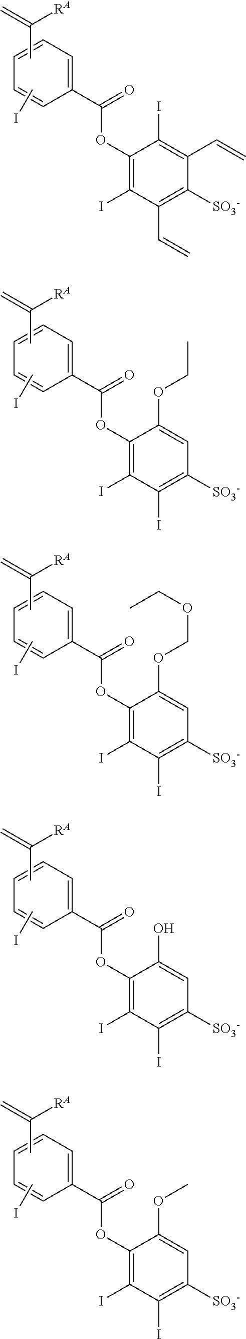

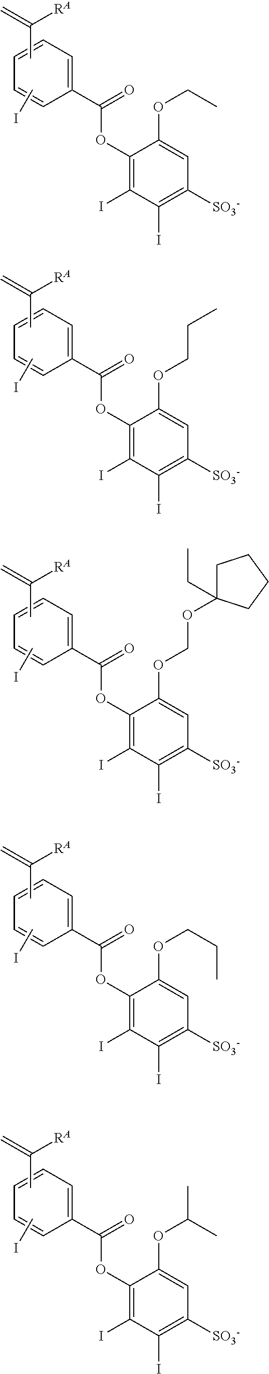

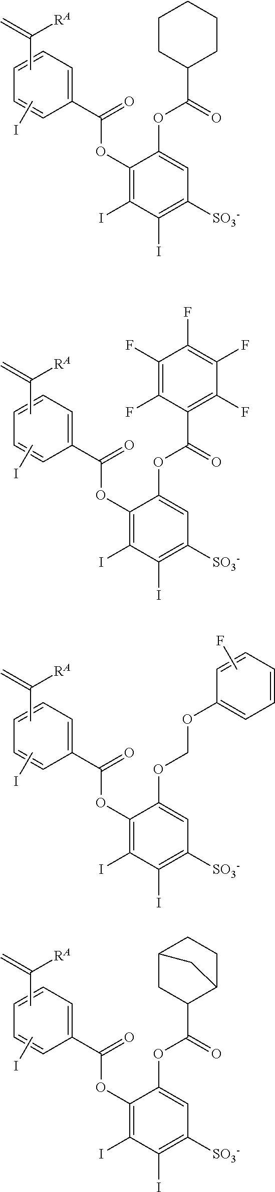

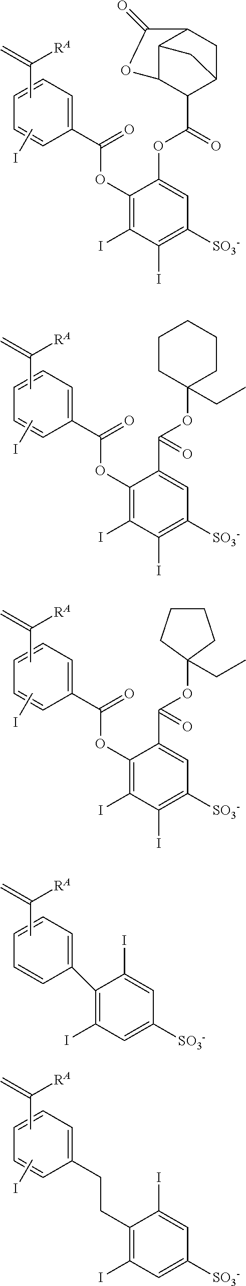

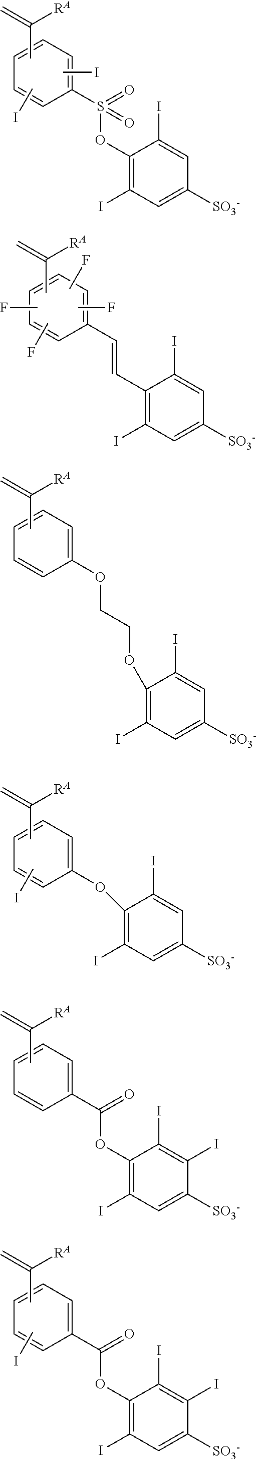

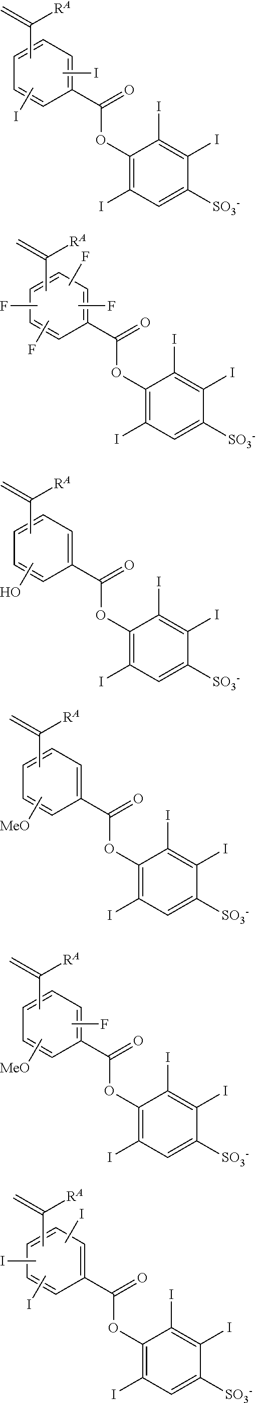

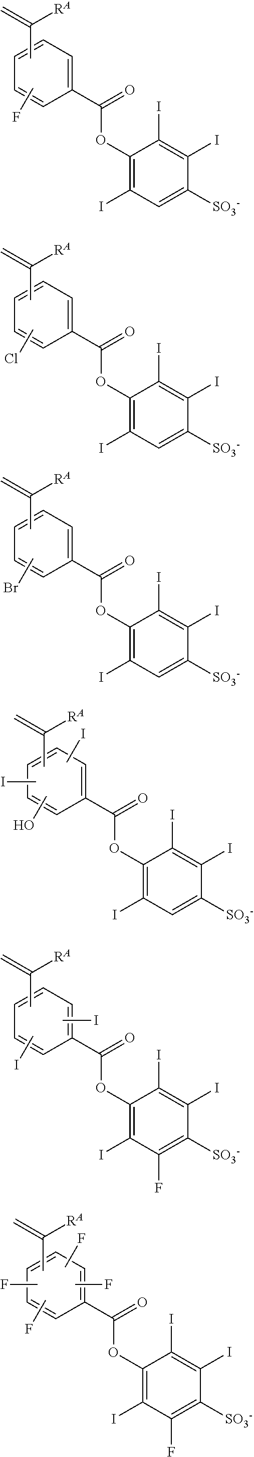

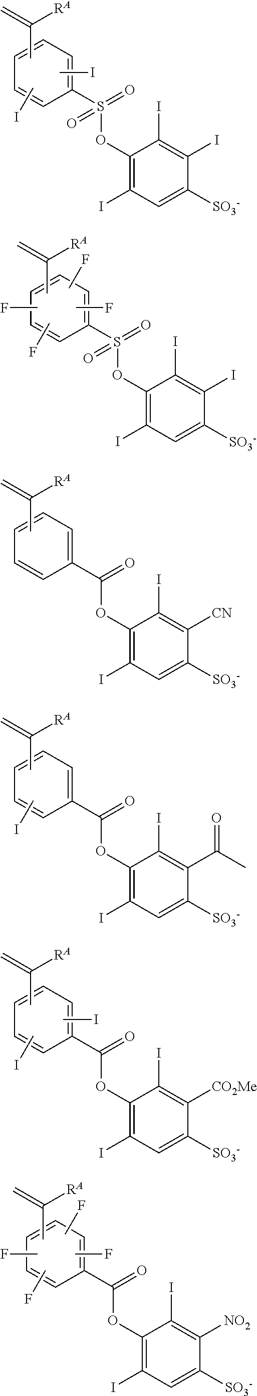

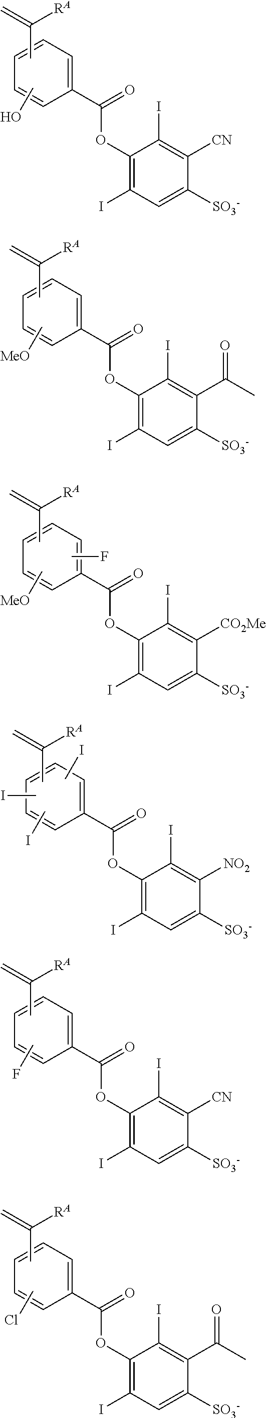

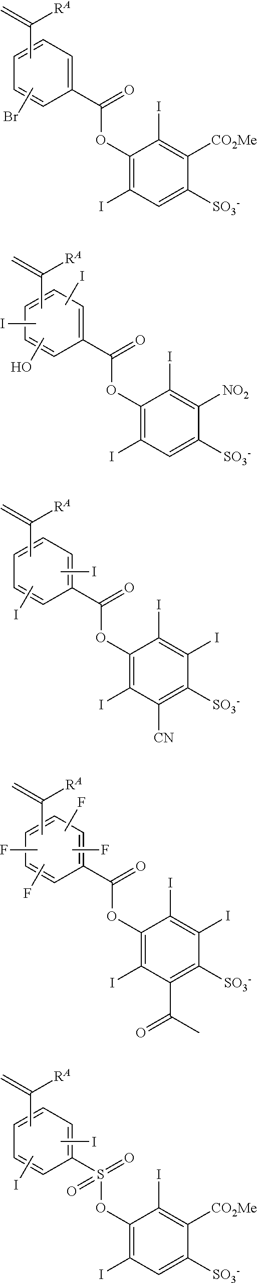

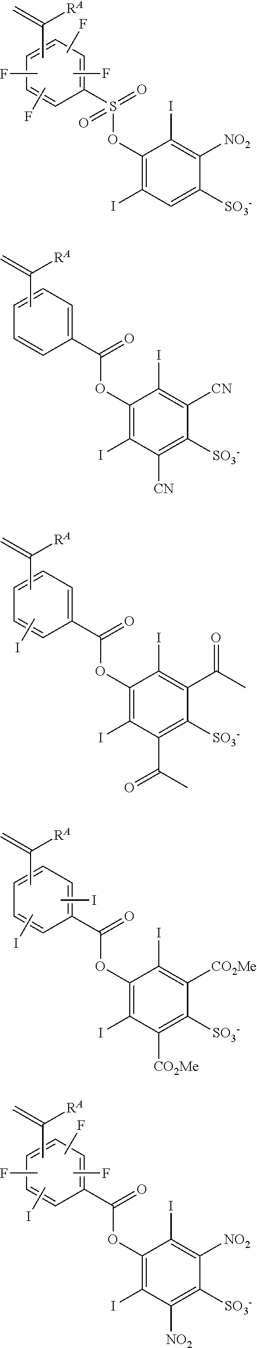

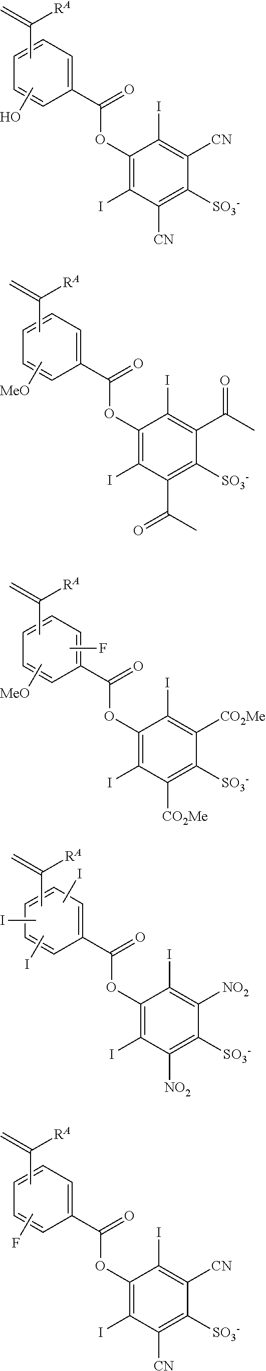

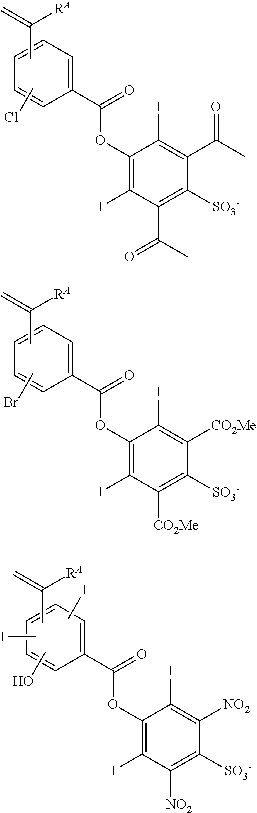

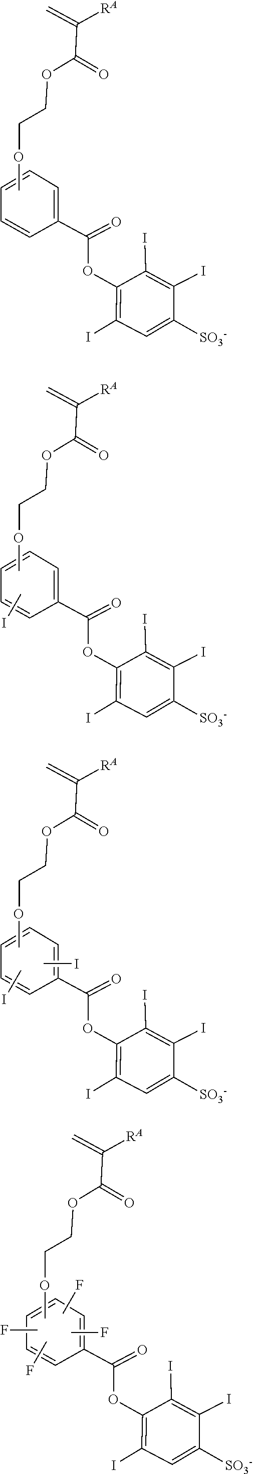

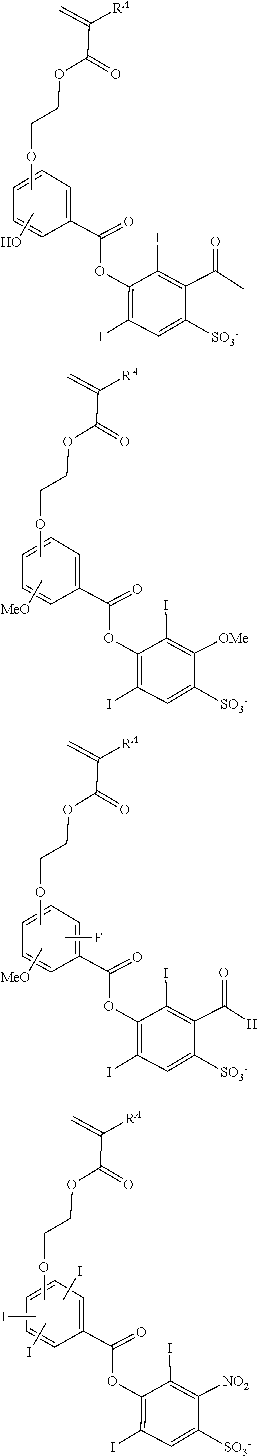

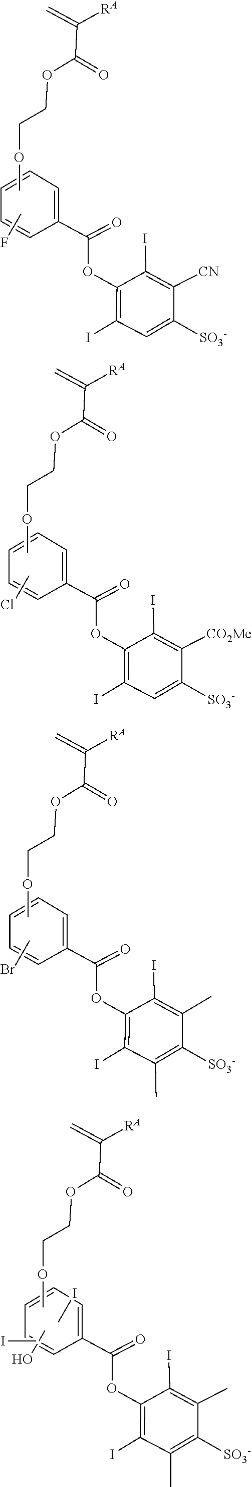

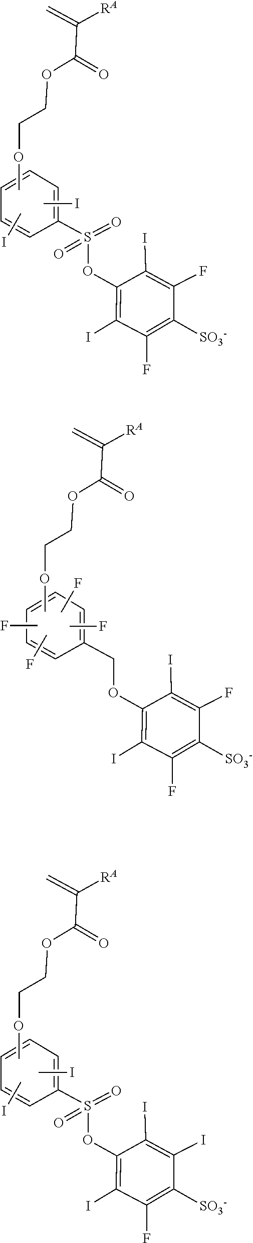

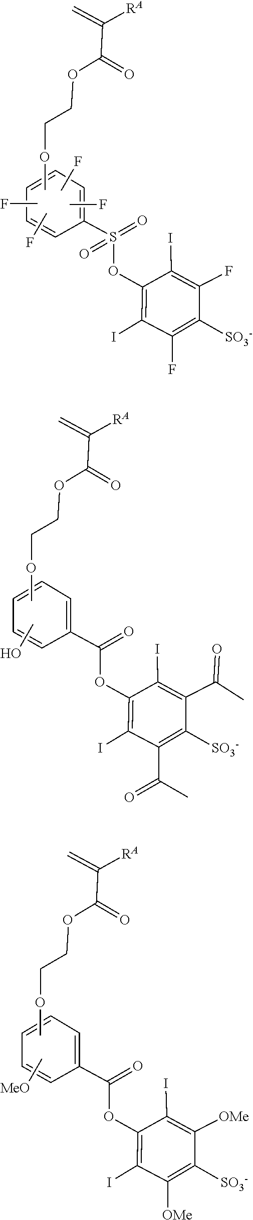

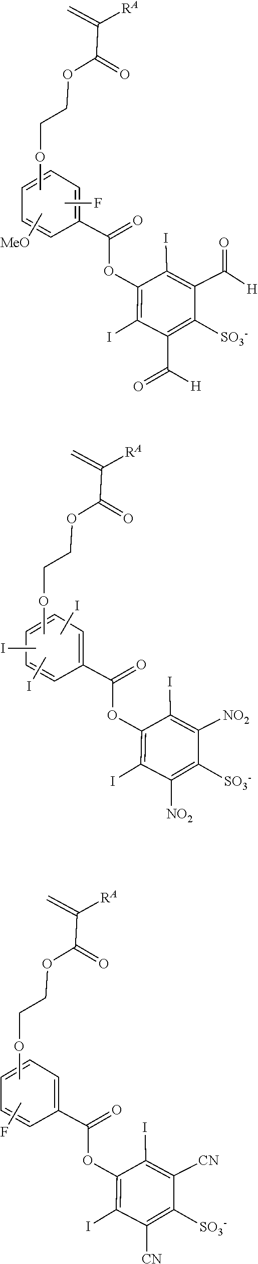

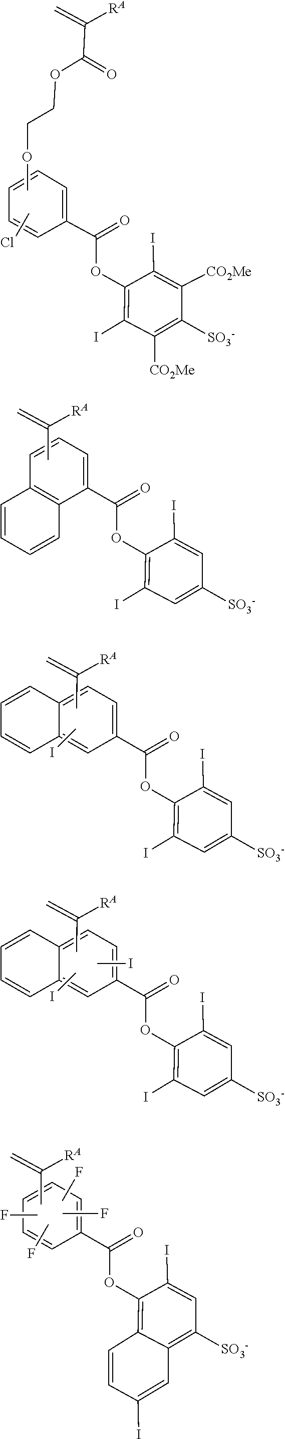

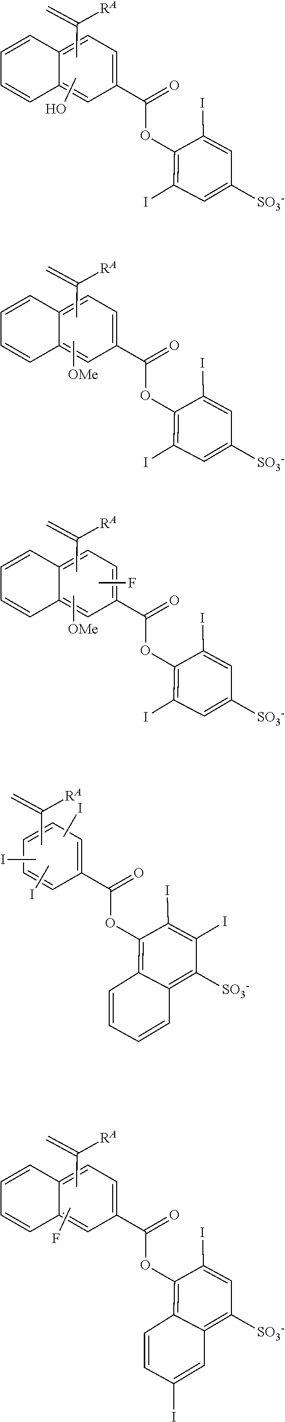

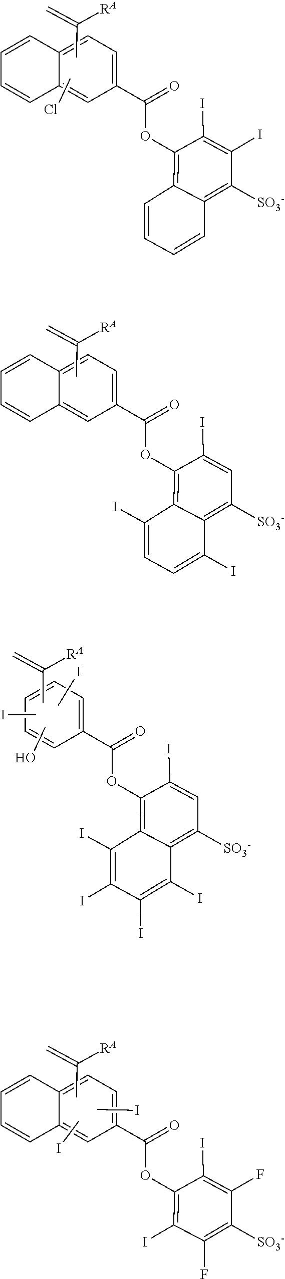

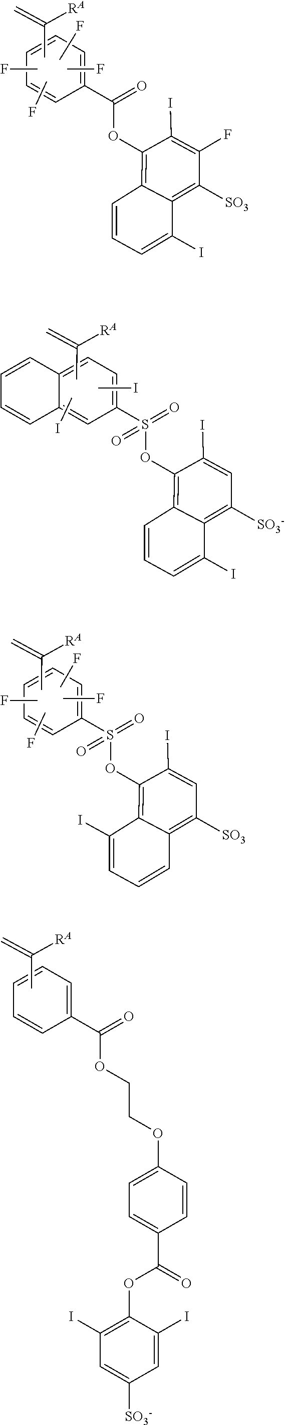

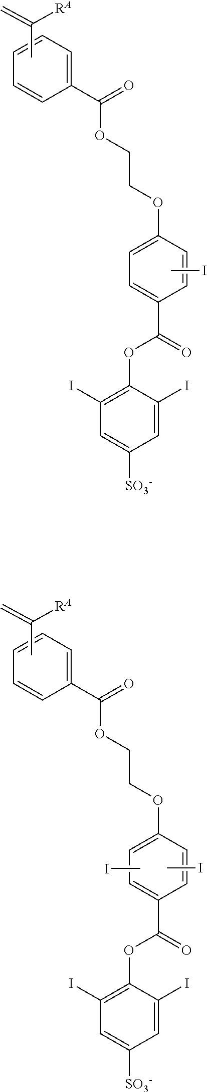

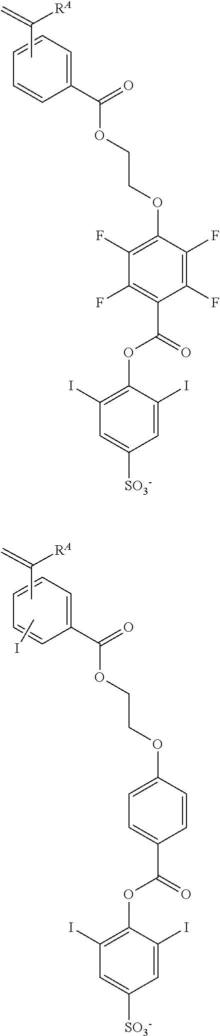

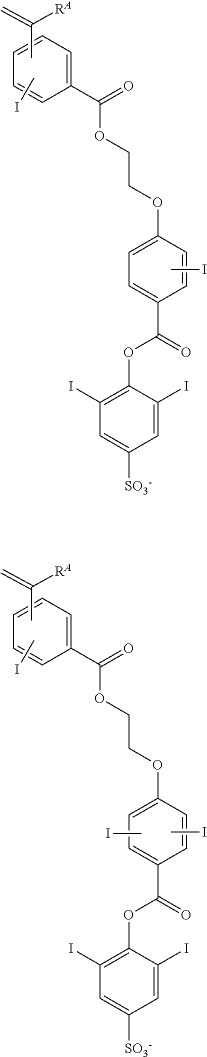

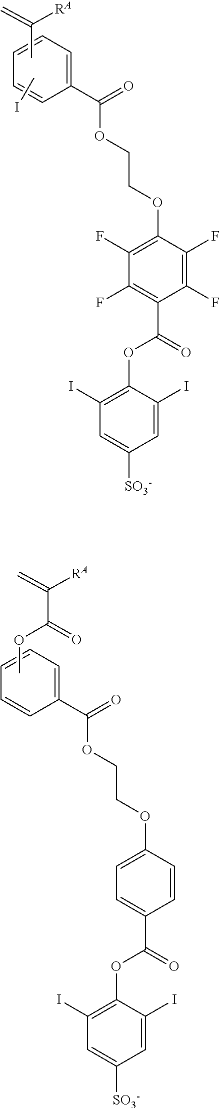

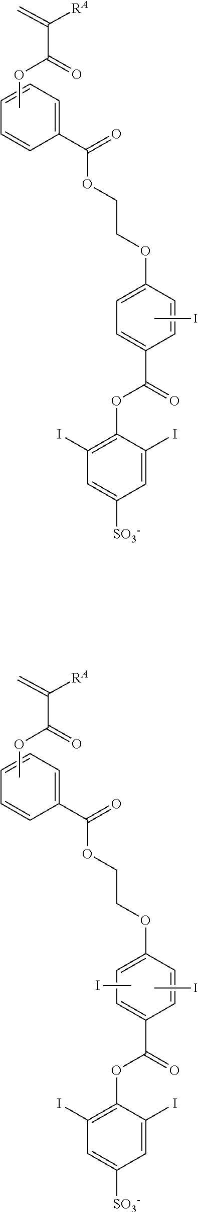

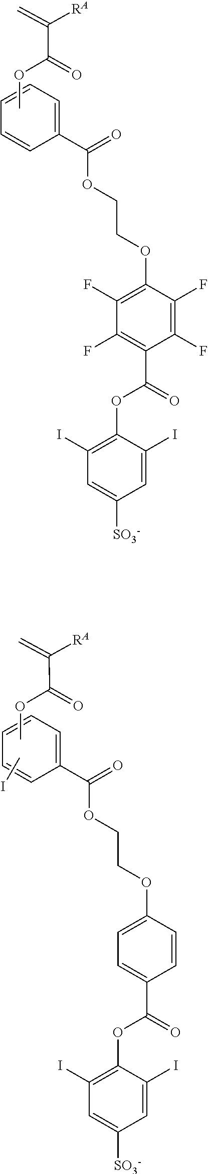

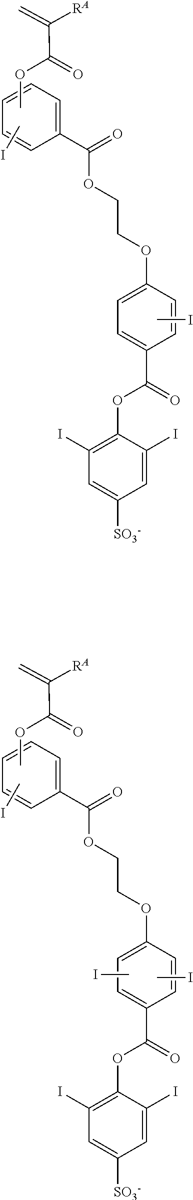

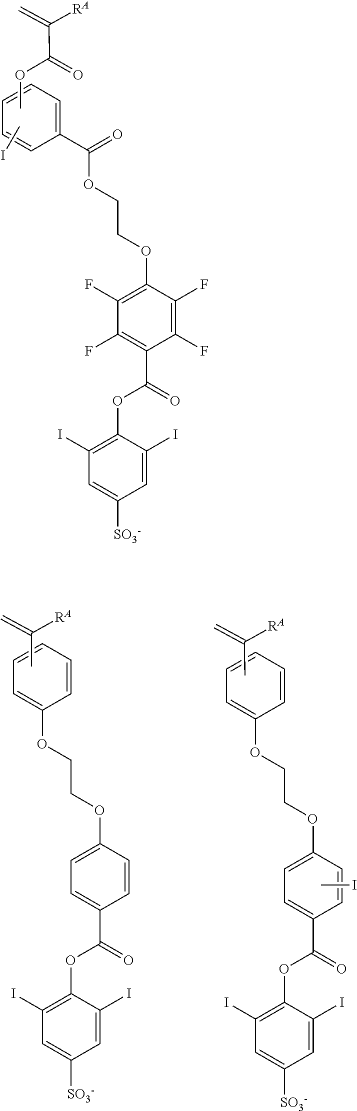

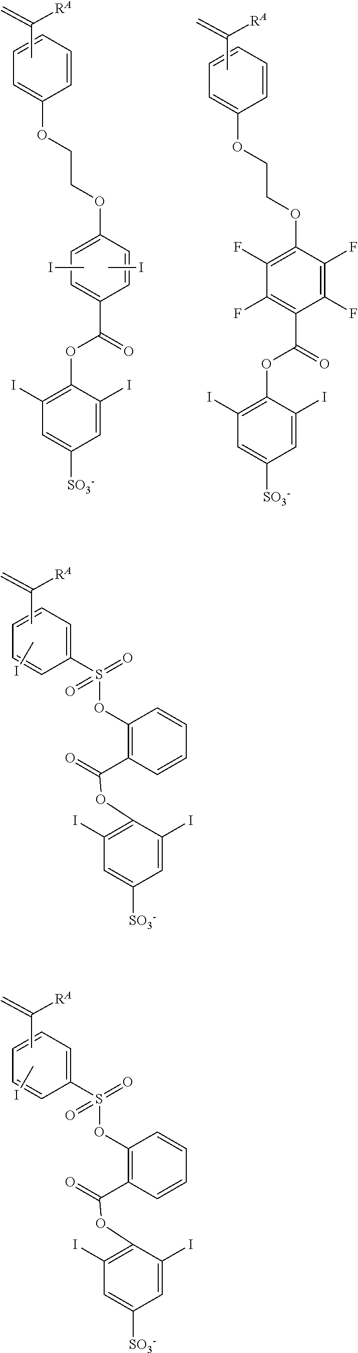

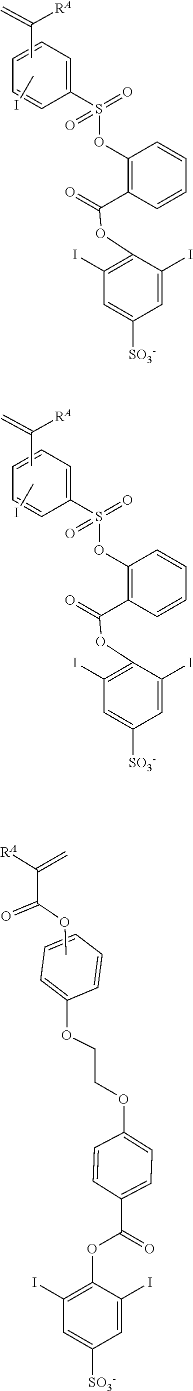

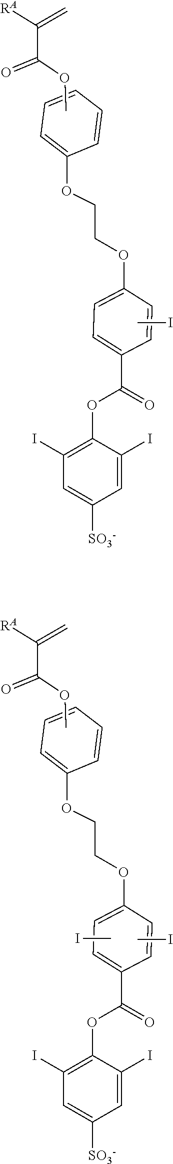

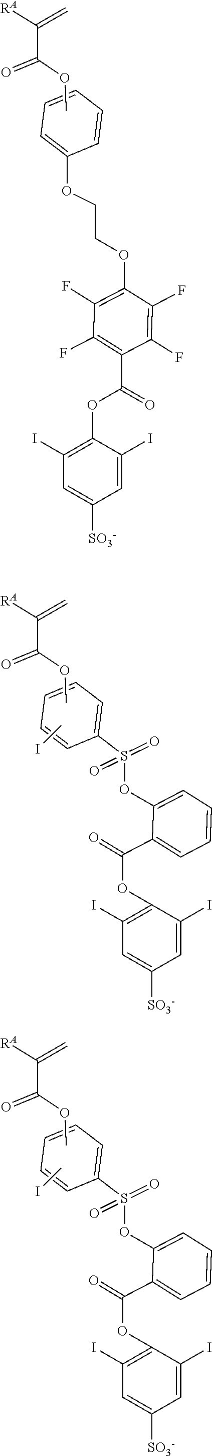

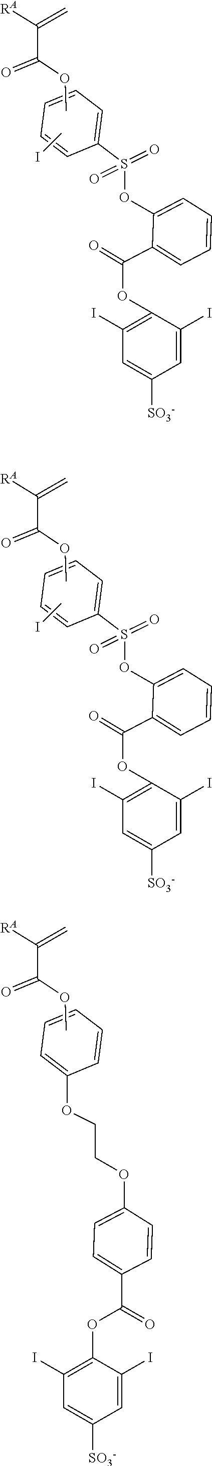

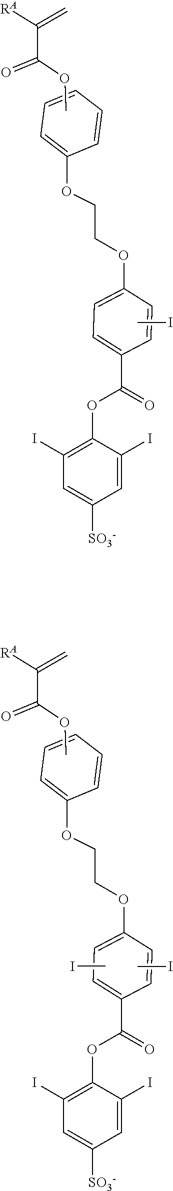

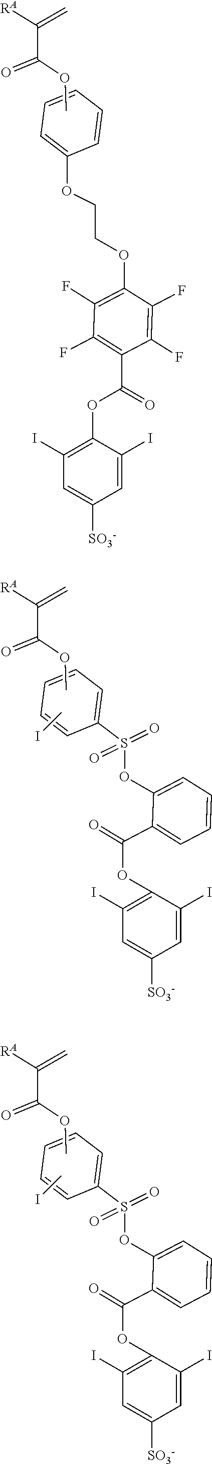

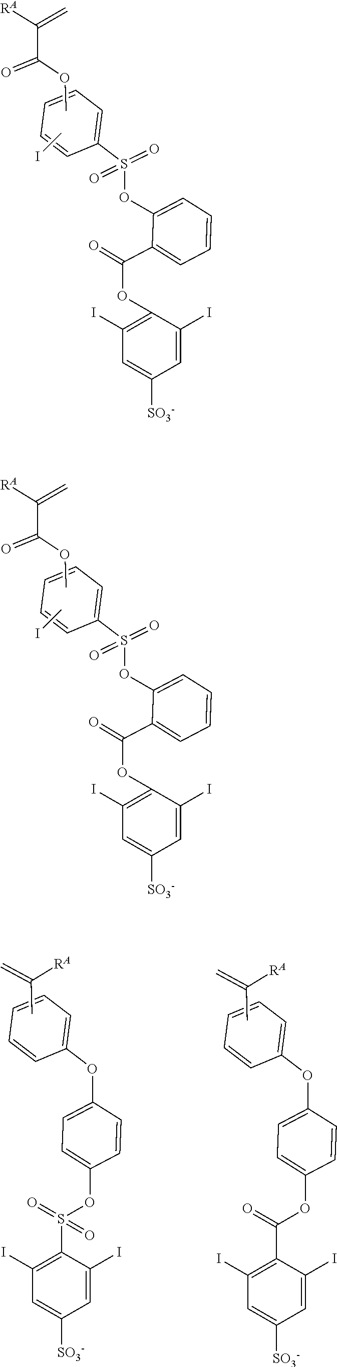

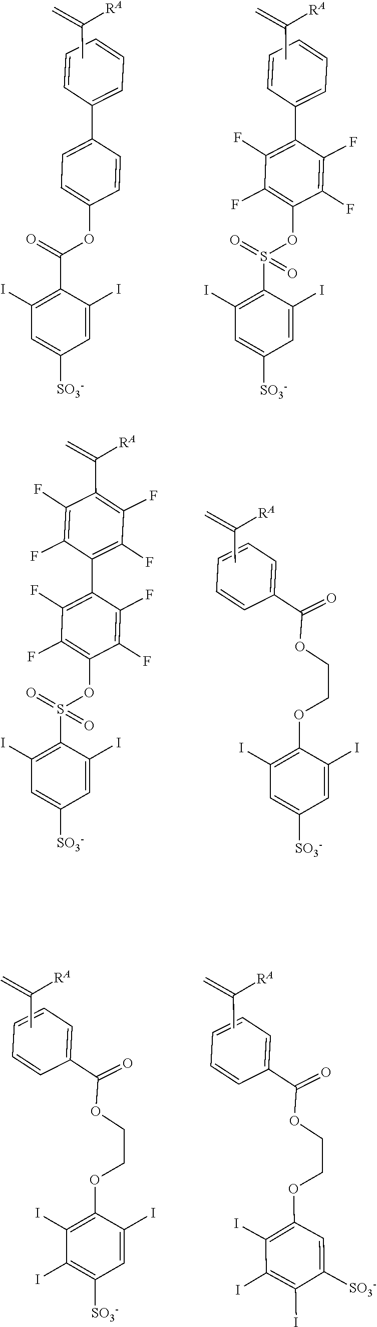

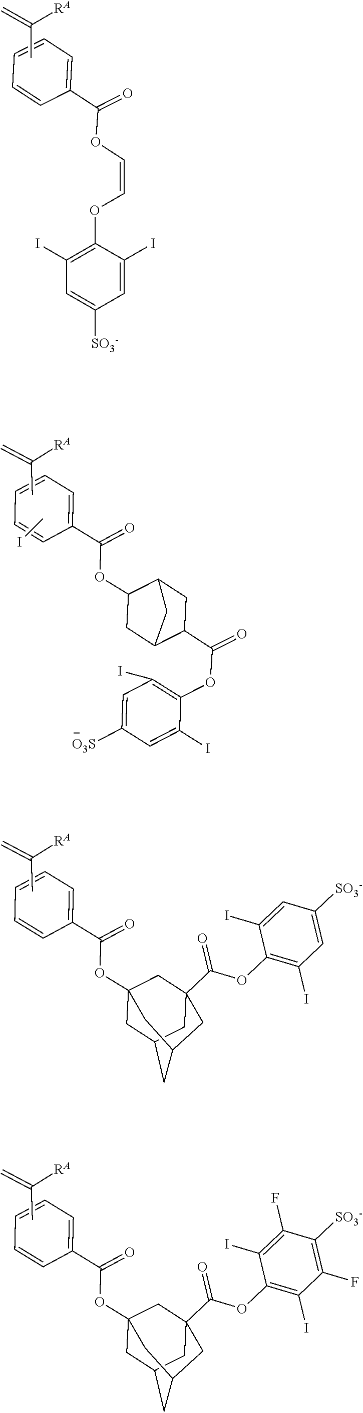

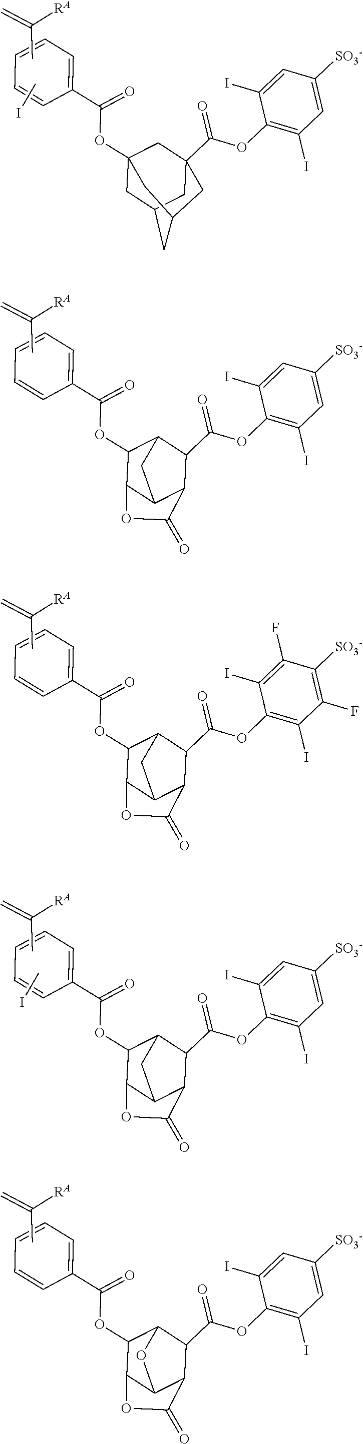

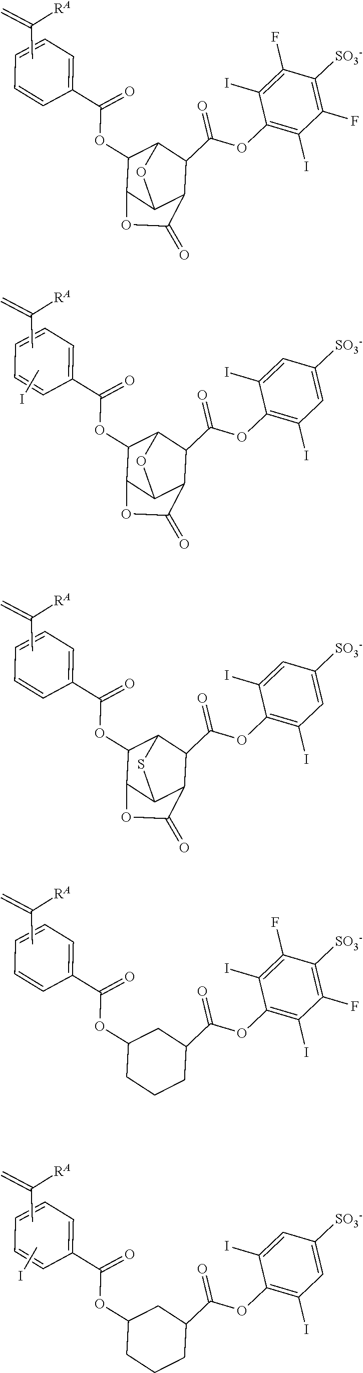

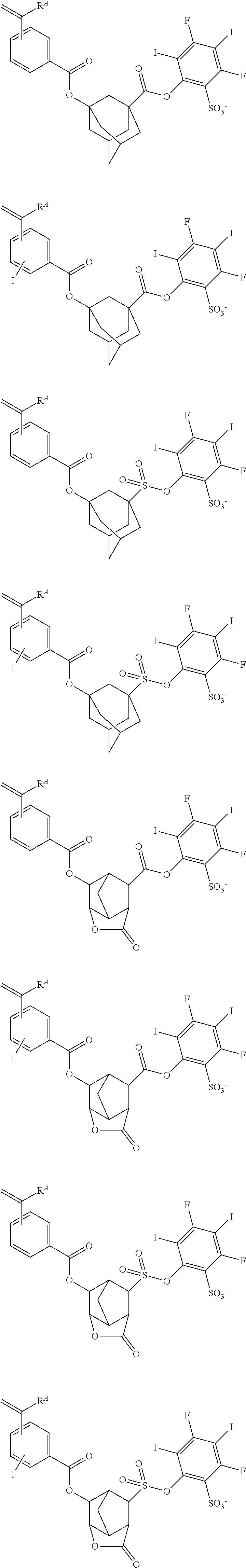

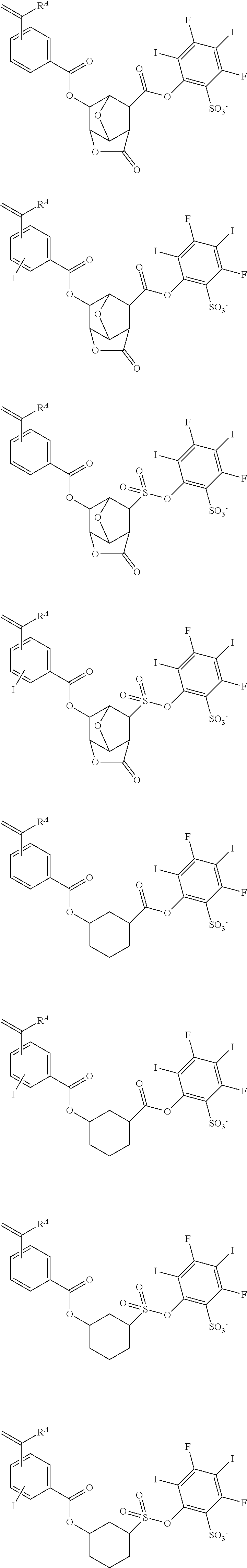

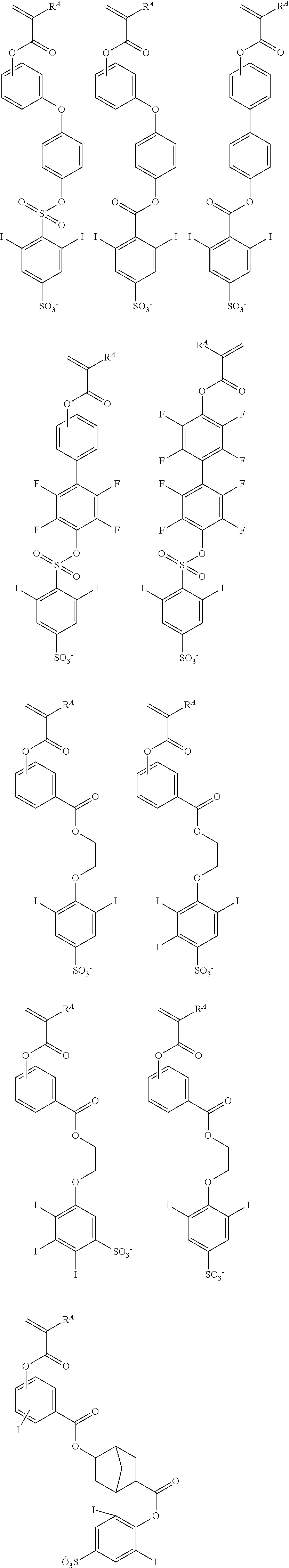

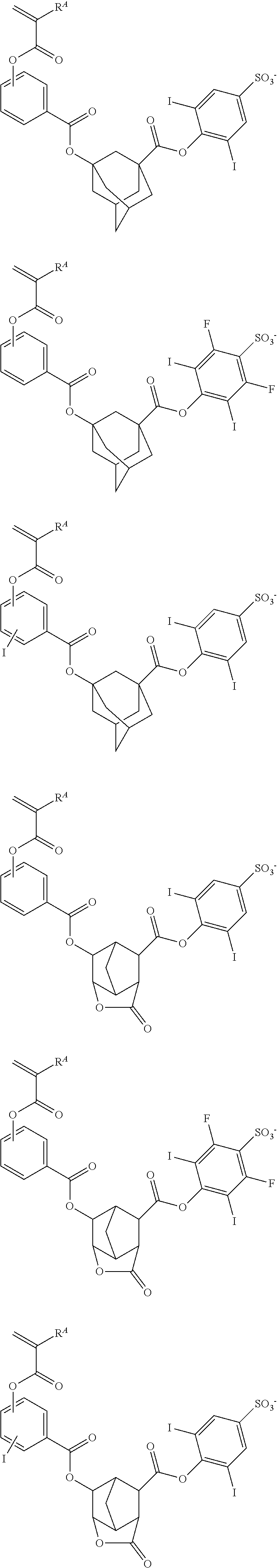

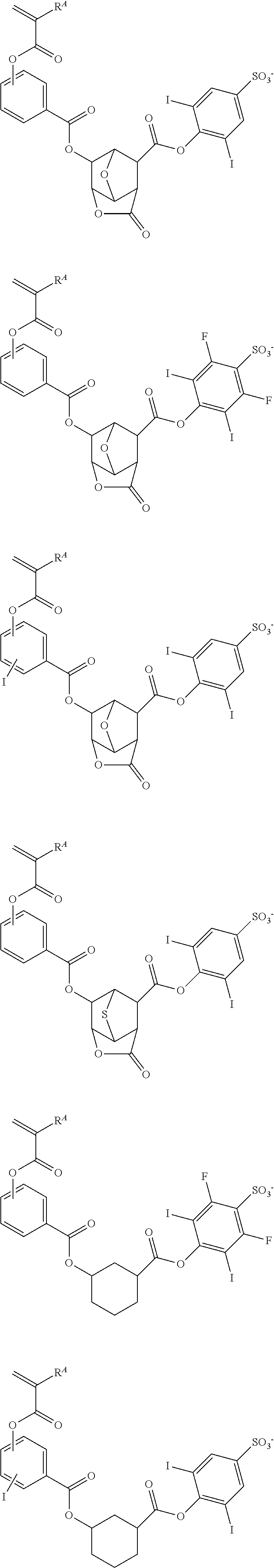

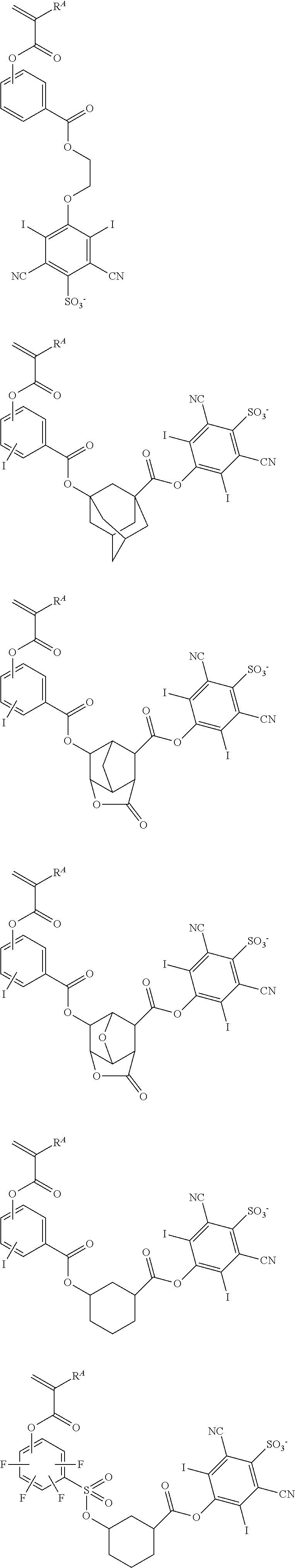

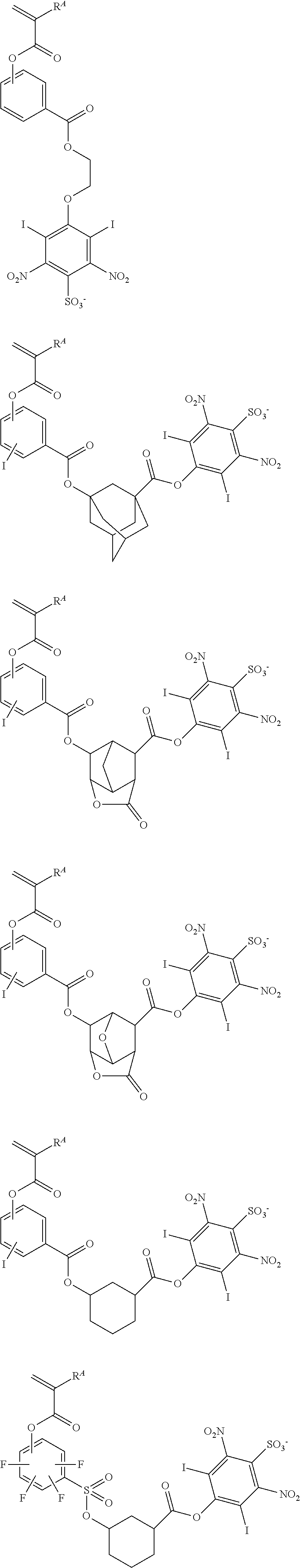

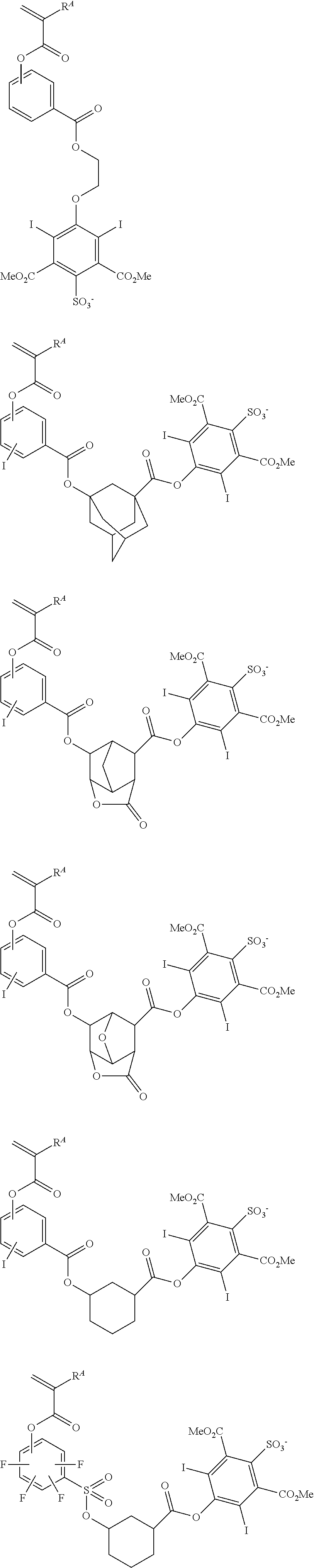

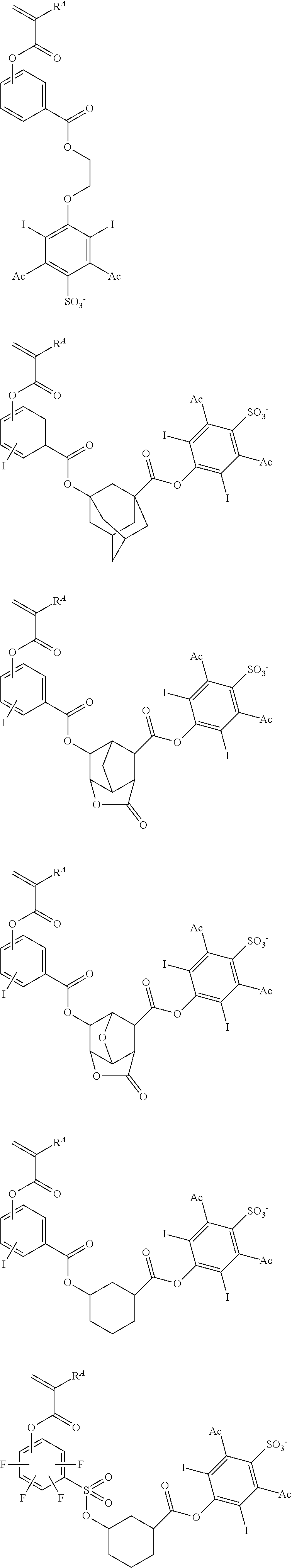

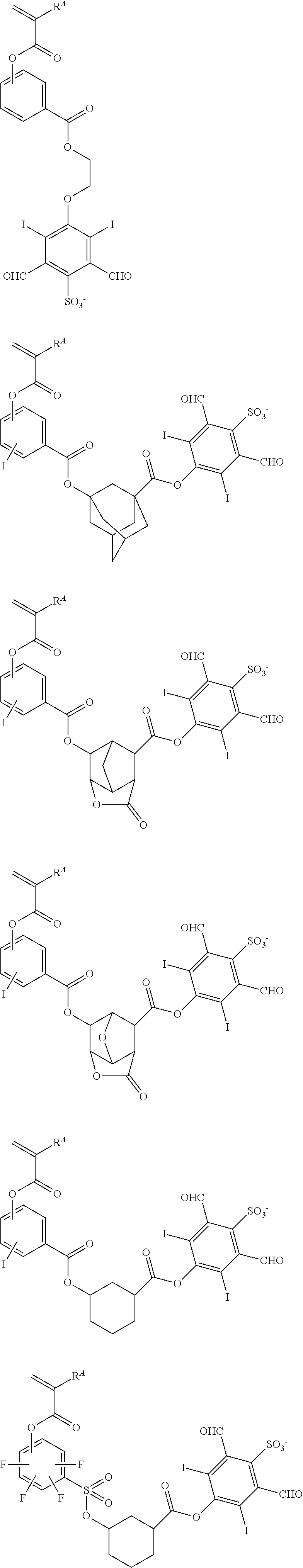

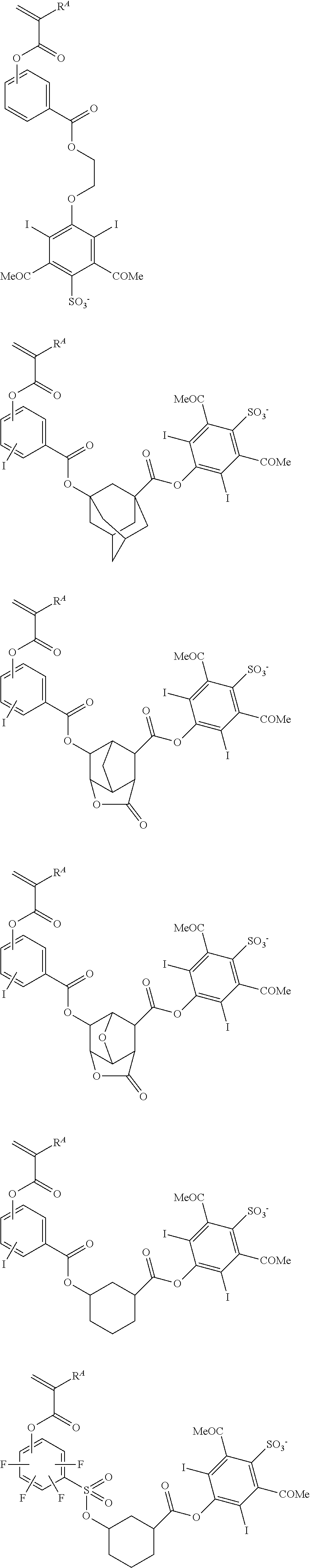

In the formula (A), Z⁺ represents an onium cation. As the onium cation, a sulfonium cation represented by the following formula (Z-1) or an iodonium cation represented by the following formula (Z-2) is preferable.

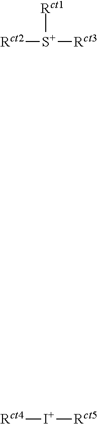

In the formulae (Z-1) and (Z-2), R^{ct1} to R^{ct5} each independently represent a halogen atom or a hydrocarbyl group having 1 to 30 carbon atoms and optionally containing a heteroatom.

Specific examples of the halogen atom represented by R^{ct1} to R^{ct5} include a fluorine atom, a chlorine atom, a bromine atom, and an iodine atom.

The hydrocarbyl group represented by R^{ct1} to R^{ct5} may be saturated or unsaturated, and may be linear, branched, or cyclic. Specific examples thereof include: alkyl groups having 1 to 30 carbon atoms, such as a methyl group, an ethyl group, an n-propyl group, an isopropyl group, an n-butyl group, an isobutyl group, a sec-butyl group, and a tert-butyl group; cyclic saturated hydrocarbyl groups having 3 to 30 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 30 carbon atoms, such as a vinyl group, an allyl group, a propenyl group, a butenyl group, and a hexenyl group; cyclic unsaturated hydrocarbyl groups having 3 to 30 carbon atoms, such as a cyclohexenyl group; aryl groups having 6 to 30 carbon atoms, such as a phenyl group, a naphthyl group, and a thienyl group; aralkyl groups having 7 to 30 carbon atoms, such as a benzyl group, a 1-phenylethyl group, and a 2-phenylethyl group; groups which are combinations of these groups; etc. Aryl groups are preferable. Furthermore, part or all of the hydrogen atoms of the hydrocarbyl group may be substituted with a group containing a heteroatom, such as an oxygen atom, a sulfur atom, a nitrogen atom, and a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Furthermore, R^{ct1} and R^{ct2} may be bonded to each other to form a ring together with the sulfur atom bonded thereto. In this case, specific examples of the structure of the ring include ones represented by the following formulae.

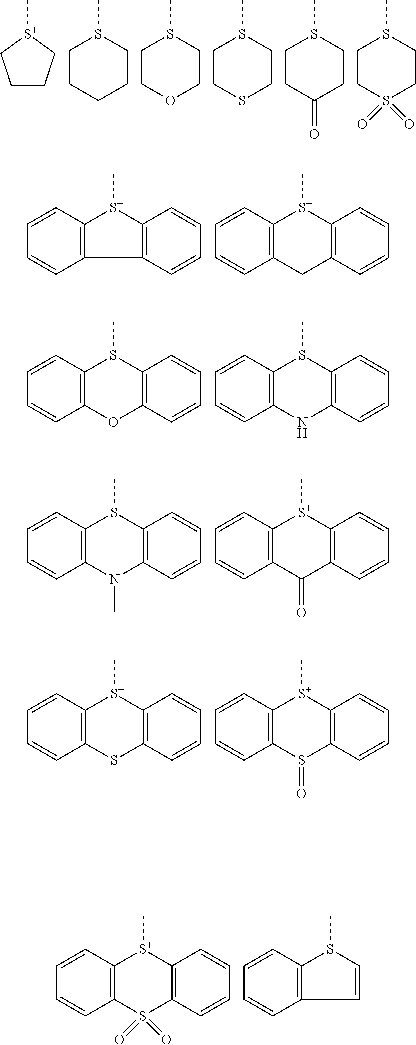

In the formulae, a broken line represents an attachment point to R^{ct3}.

Specific examples of the sulfonium cation represented by the formula (Z-1) include ones shown below, but are not limited thereto.

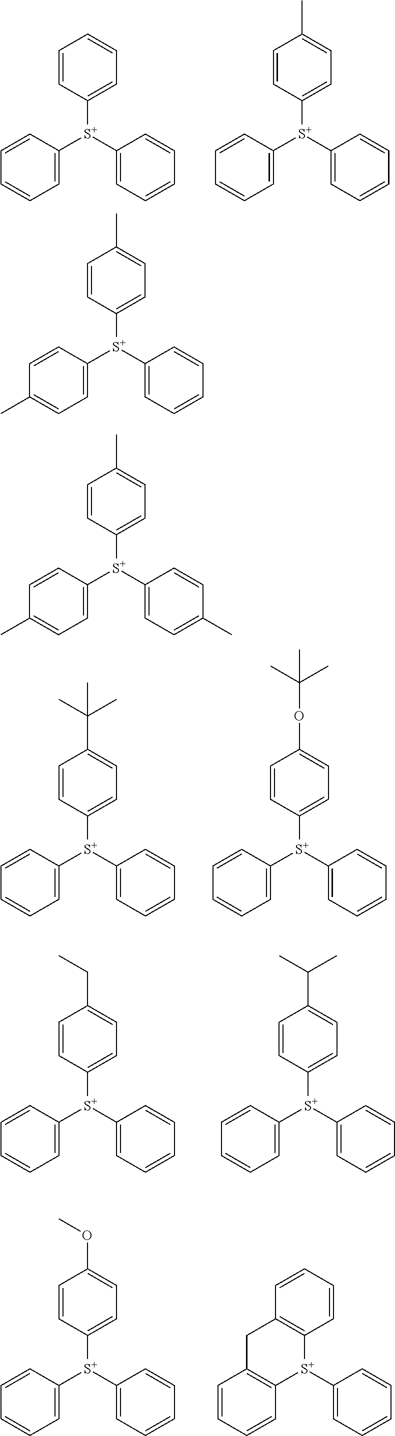

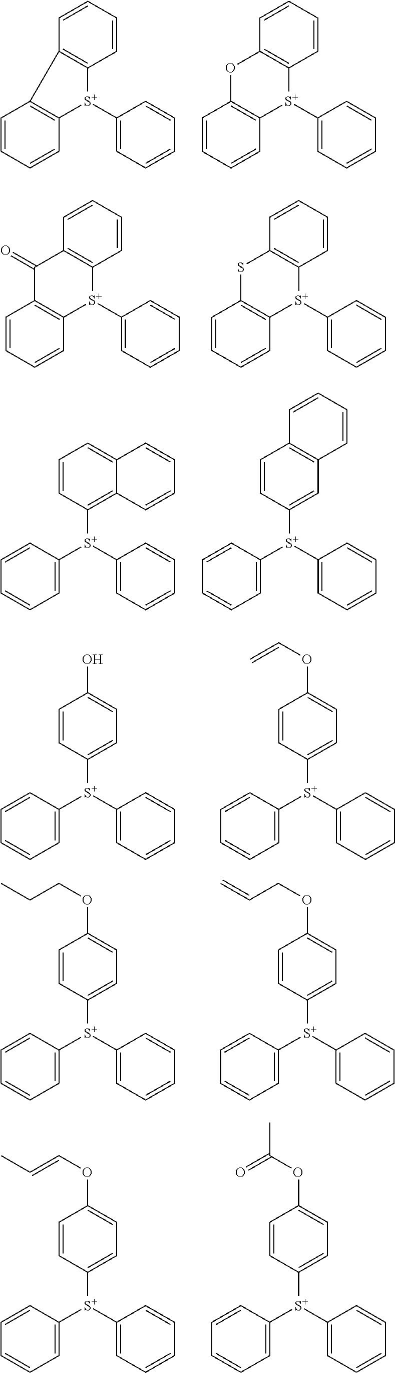

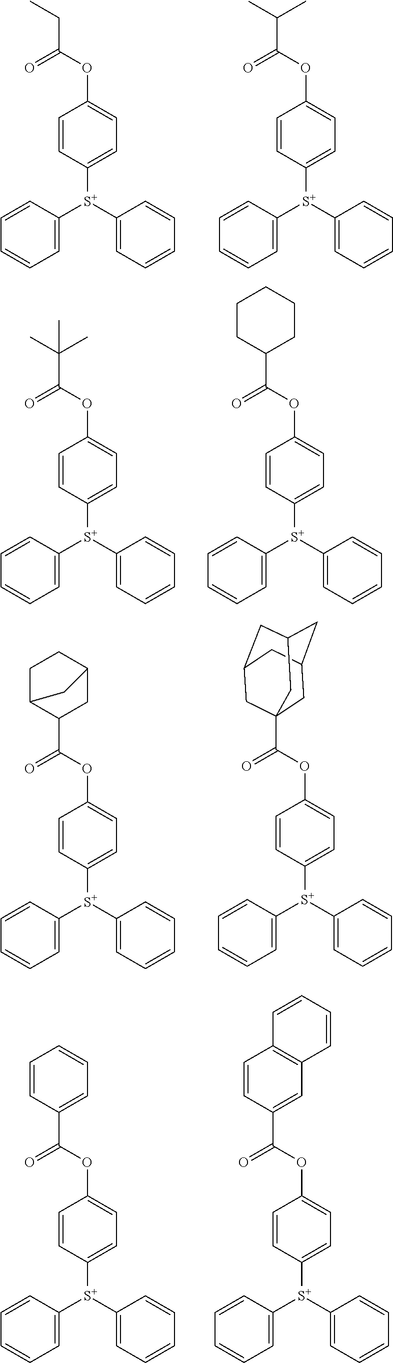

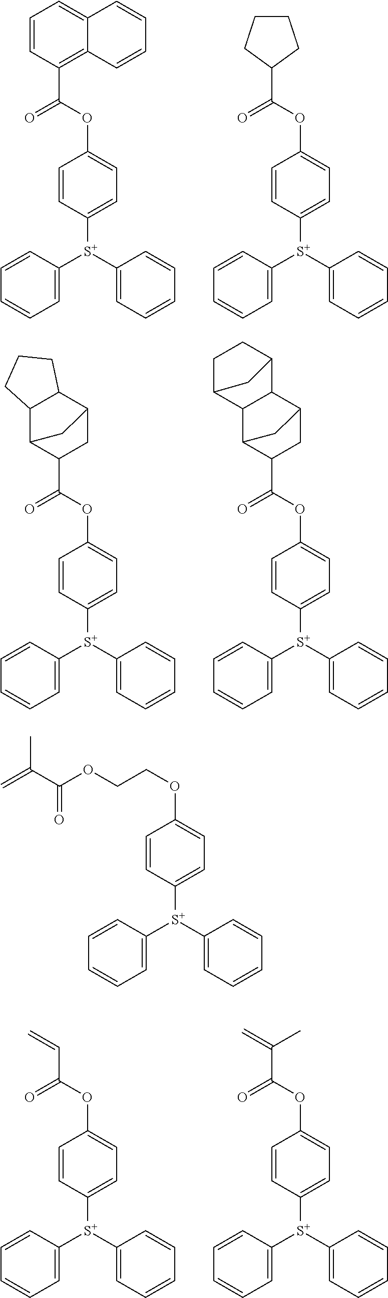

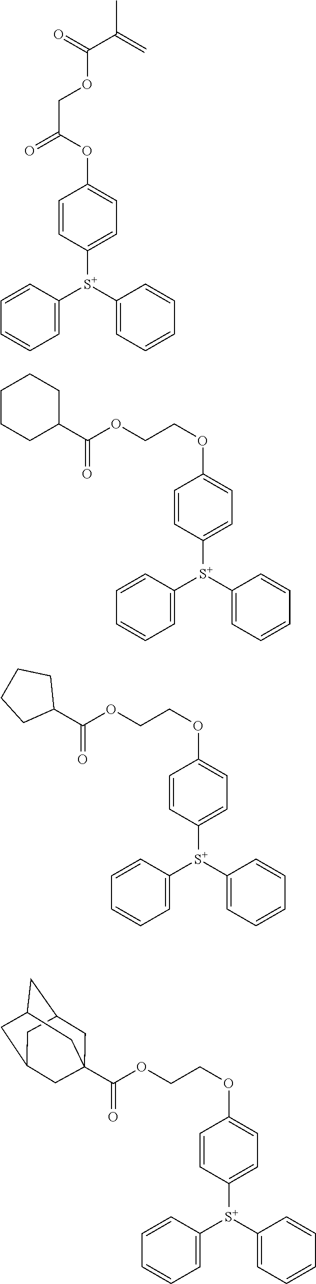 [SMILES: C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2]

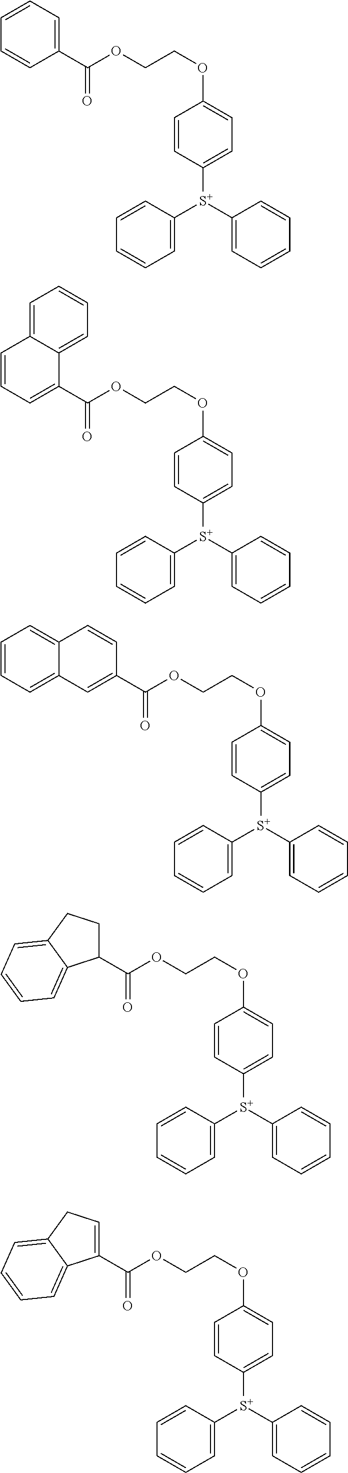 [SMILES: O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21]

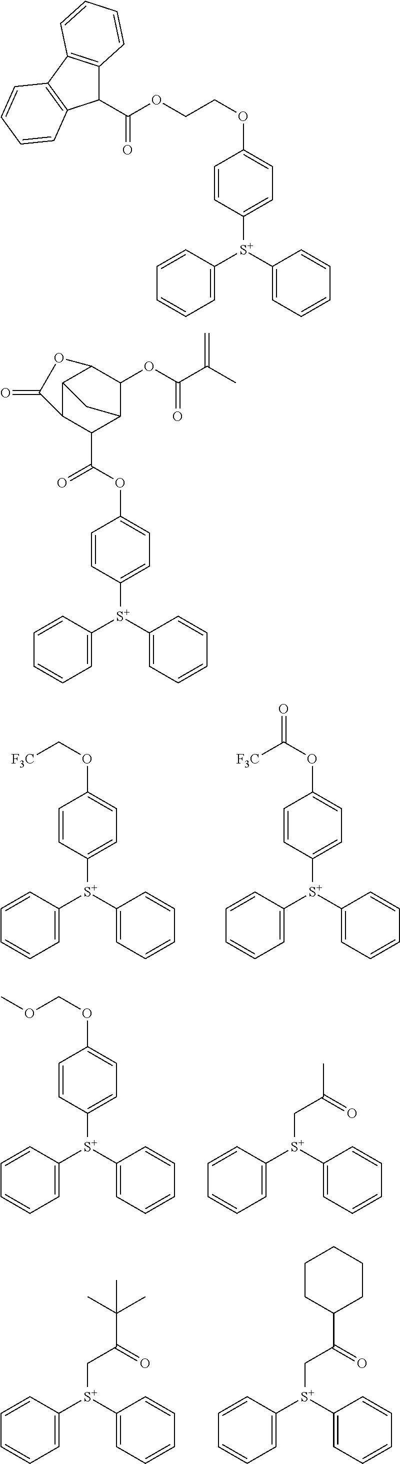

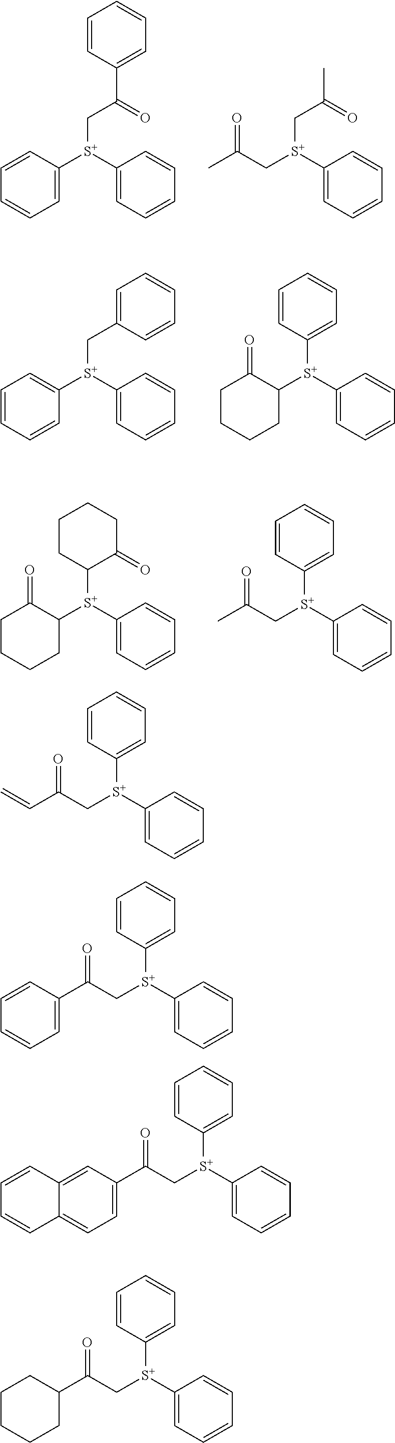

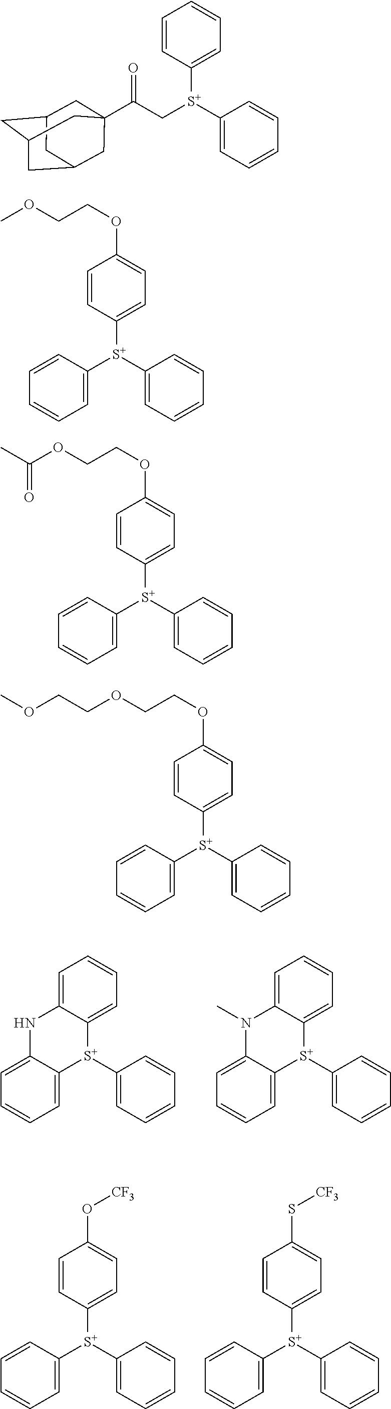

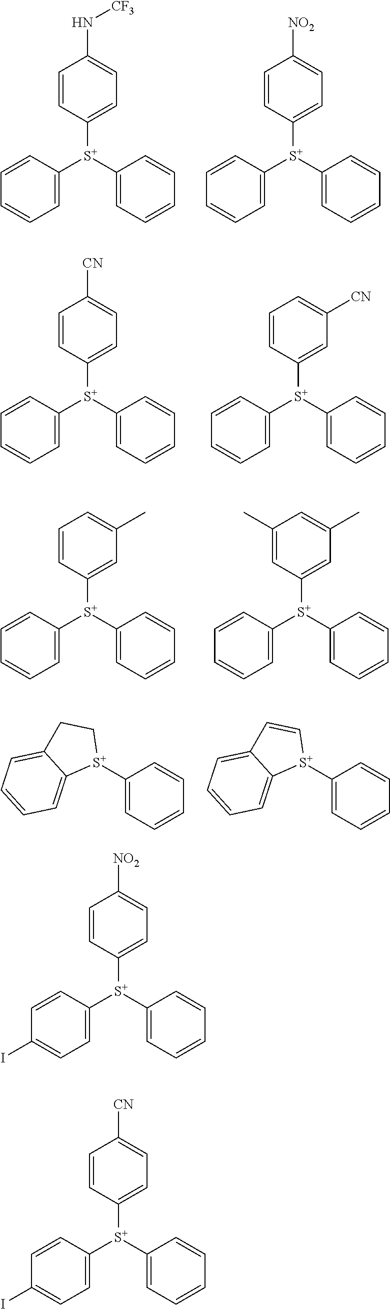

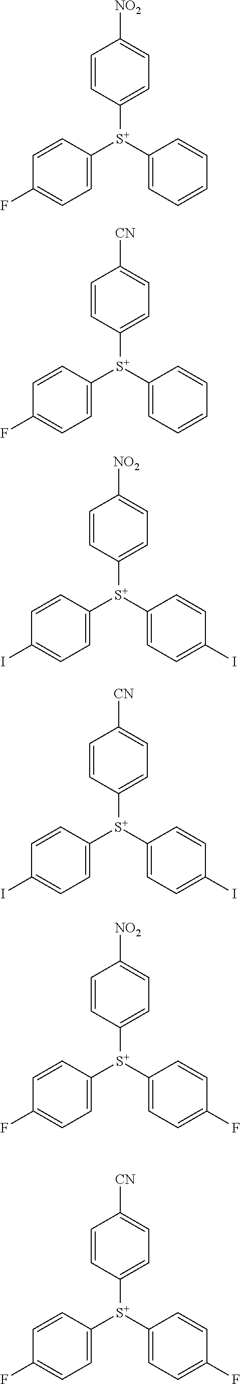 [SMILES: O=[N+]([O-])c1ccc([S+](c2ccccc2)c2ccc(F)cc2)cc1 ; N#Cc1ccc([S+](c2ccccc2)c2ccc(F)cc2)cc1 ; O=[N+]([O-])c1ccc([S+](c2ccc(I)cc2)c2ccc(I)cc2)cc1 ; N#Cc1ccc([S+](c2ccc(I)cc2)c2ccc(I)cc2)cc1 ; O=[N+]([O-])c1ccc([S+](c2ccc(F)cc2)c2ccc(F)cc2)cc1 ; N#Cc1ccc([S+](c2ccc(F)cc2)c2ccc(F)cc2)cc1]

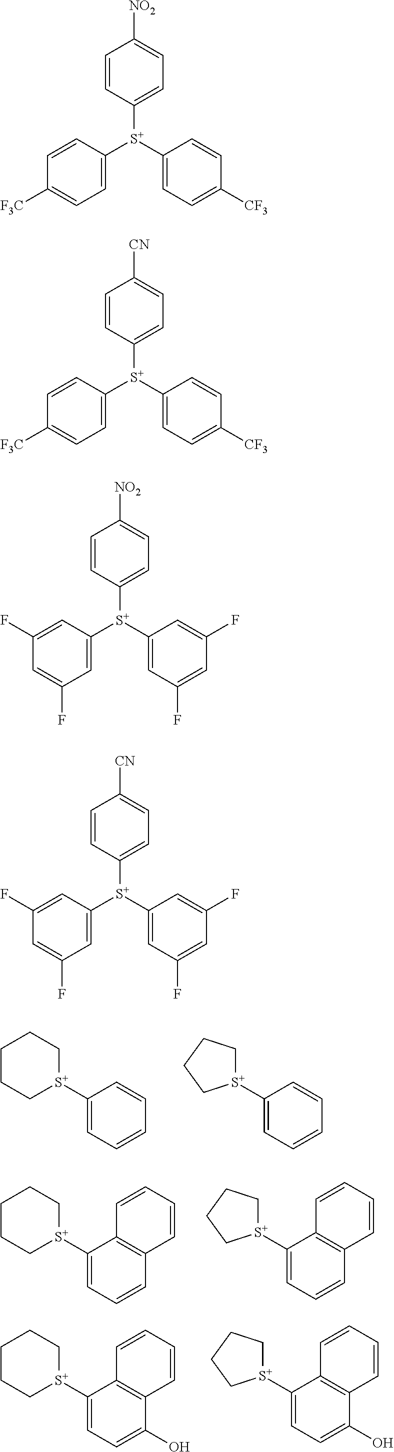

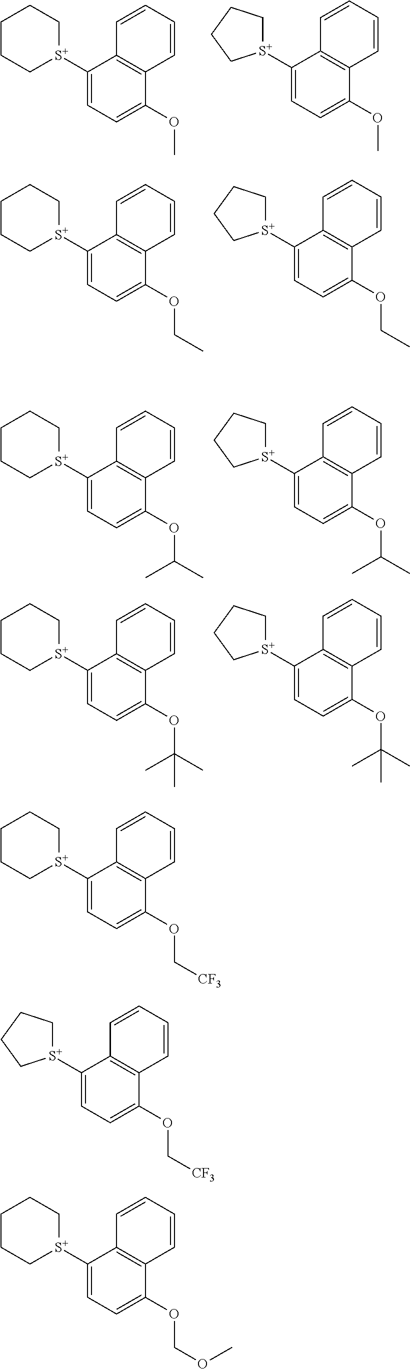

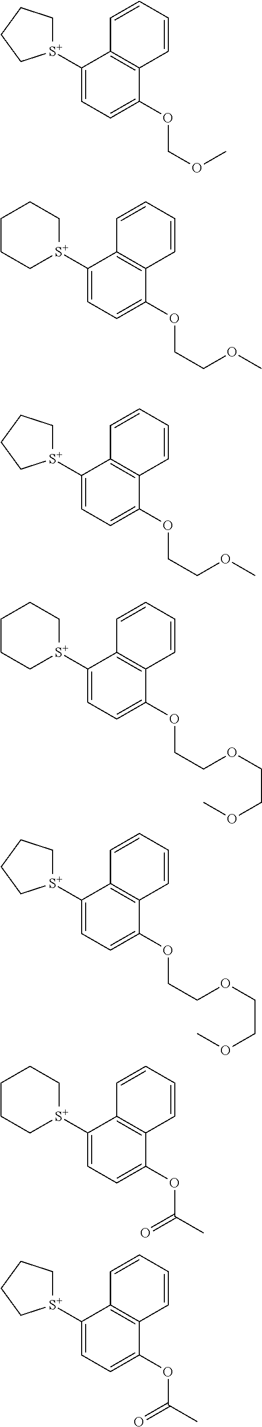

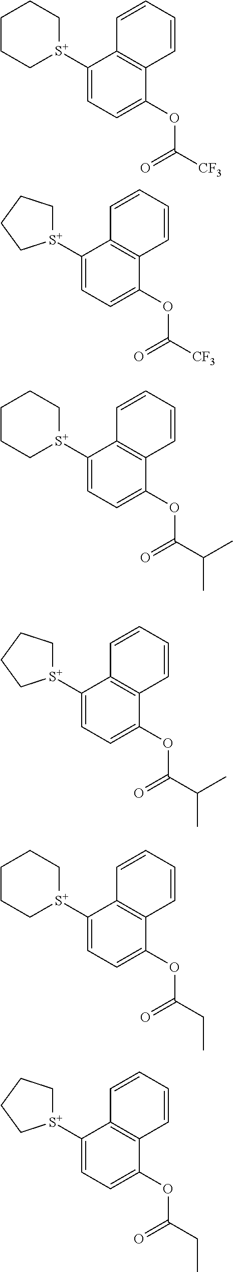 [SMILES: O=C(Oc1ccc([S+]2CCCCC2)c2ccccc12)C(F)(F)F ; O=C(Oc1ccc([S+]2CCCC2)c2ccccc12)C(F)(F)F ; CC(C)C(=O)Oc1ccc([S+]2CCCCC2)c2ccccc12 ; CC(C)C(=O)Oc1ccc([S+]2CCCC2)c2ccccc12 ; CCC(=O)Oc1ccc([S+]2CCCCC2)c2ccccc12 ; CCC(=O)Oc1ccc([S+]2CCCC2)c2ccccc12]

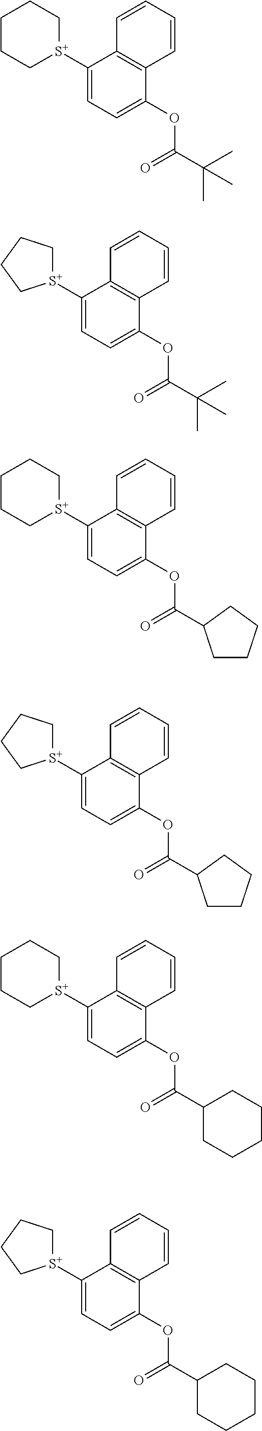 [SMILES: CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1]

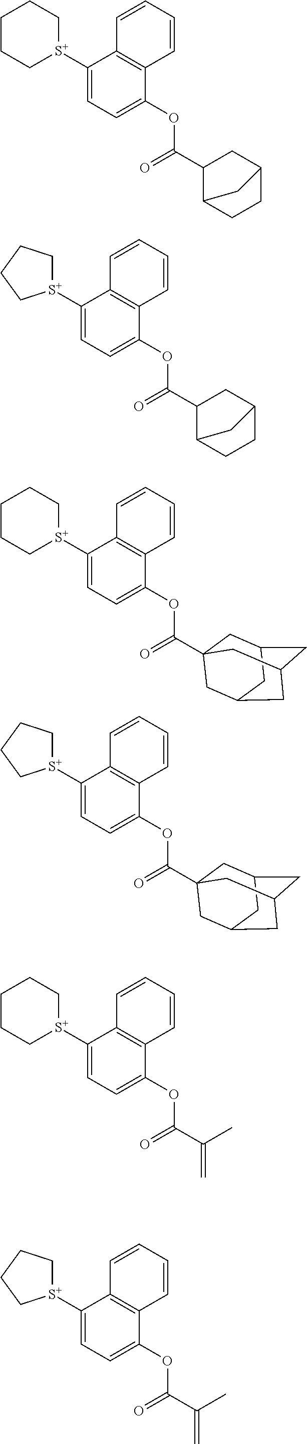 [SMILES: O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12]

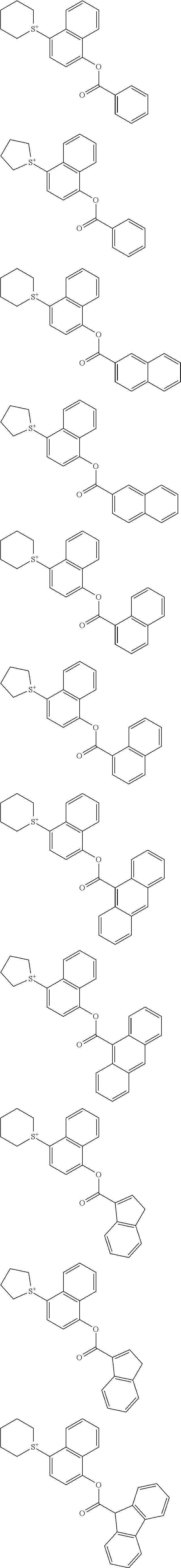

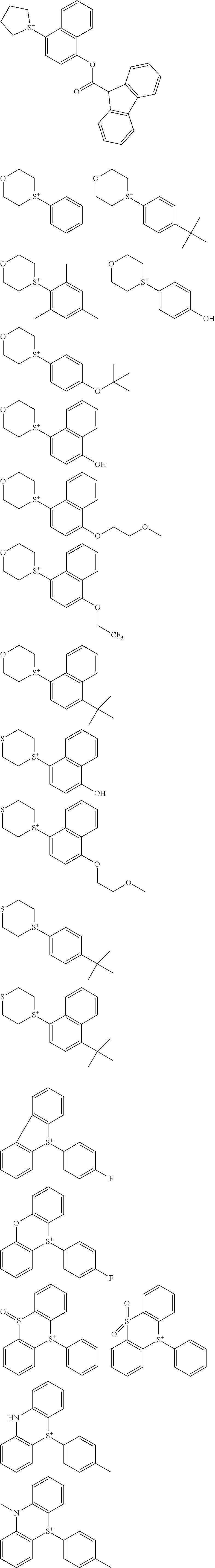

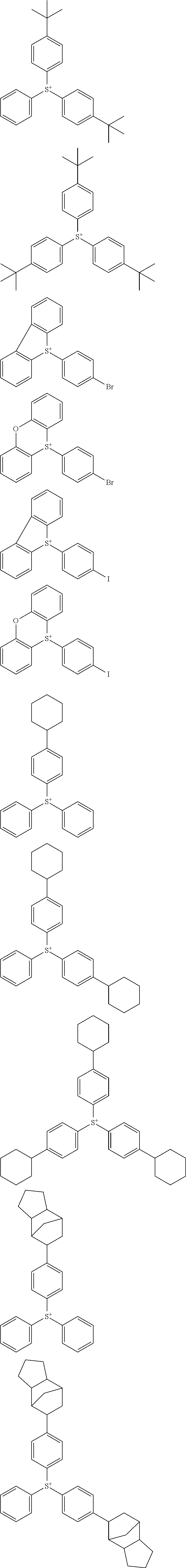

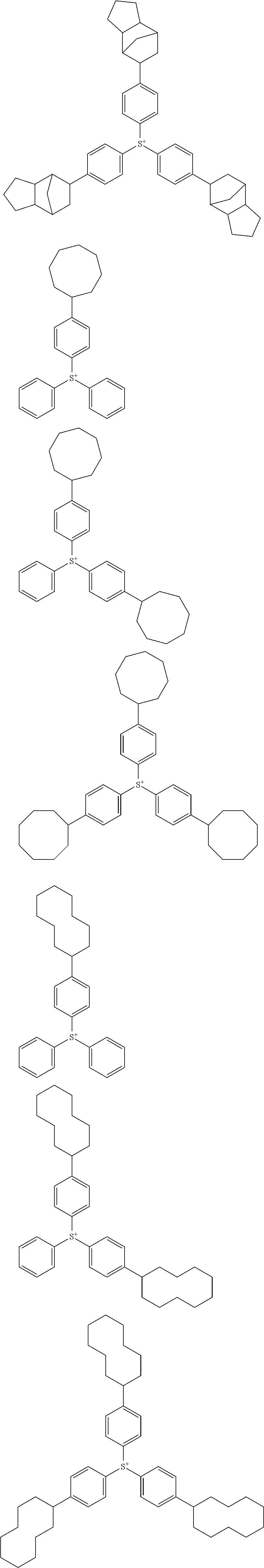

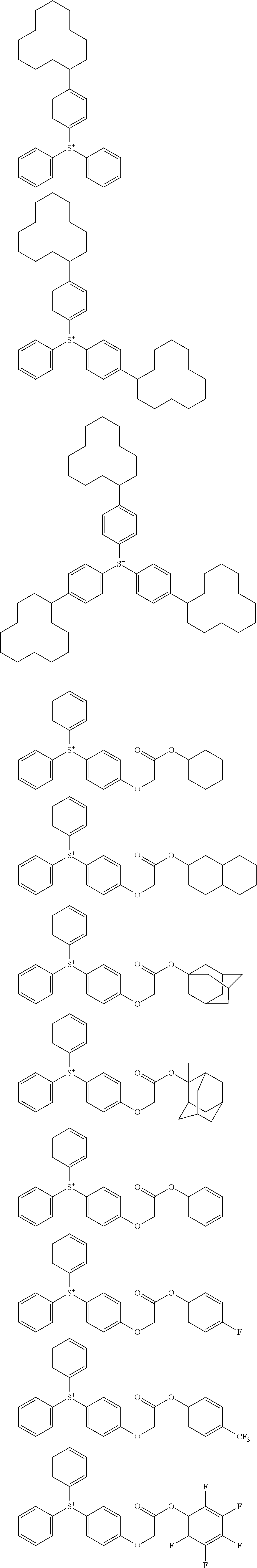

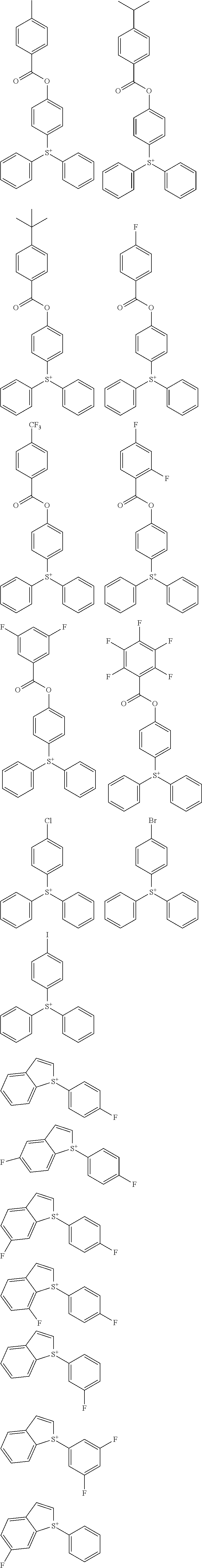

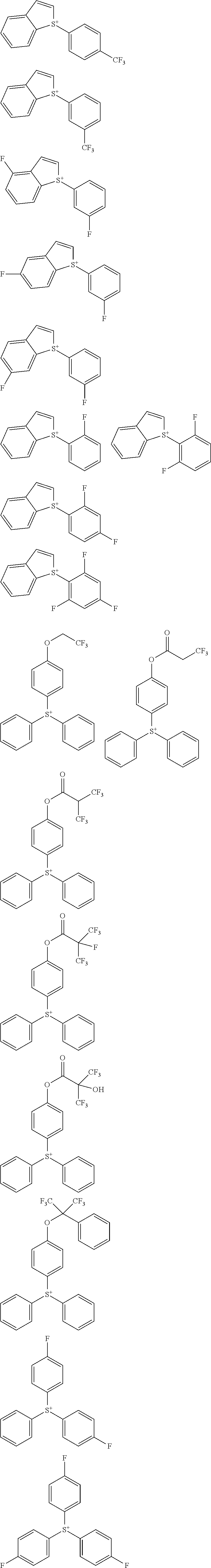

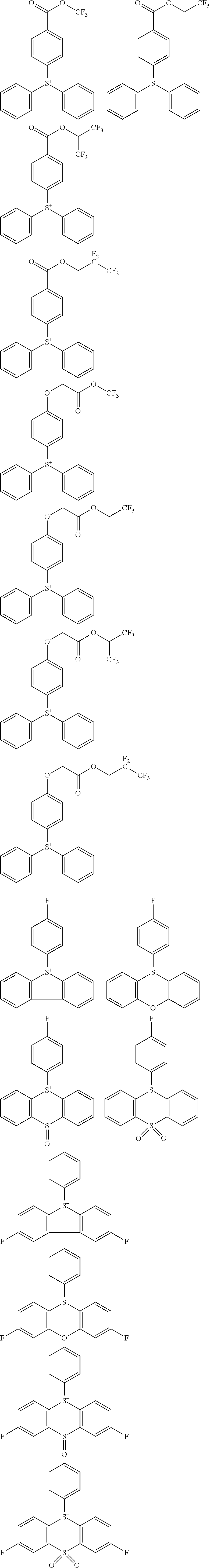

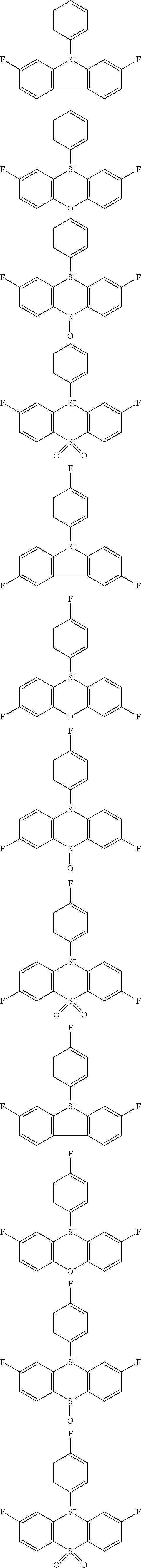

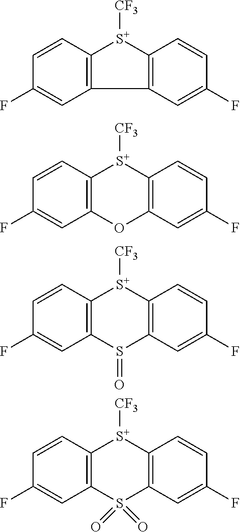 [SMILES: Fc1ccc2c(c1)c1cc(F)ccc1[s+]2C(F)(F)F ; Fc1ccc2c(c1)Oc1cc(F)ccc1[S+]2C(F)(F)F ; O=S1c2cc(F)ccc2[S+](C(F)(F)F)c2ccc(F)cc21 ; O=S1(=O)c2cc(F)ccc2[S+](C(F)(F)F)c2ccc(F)cc21]

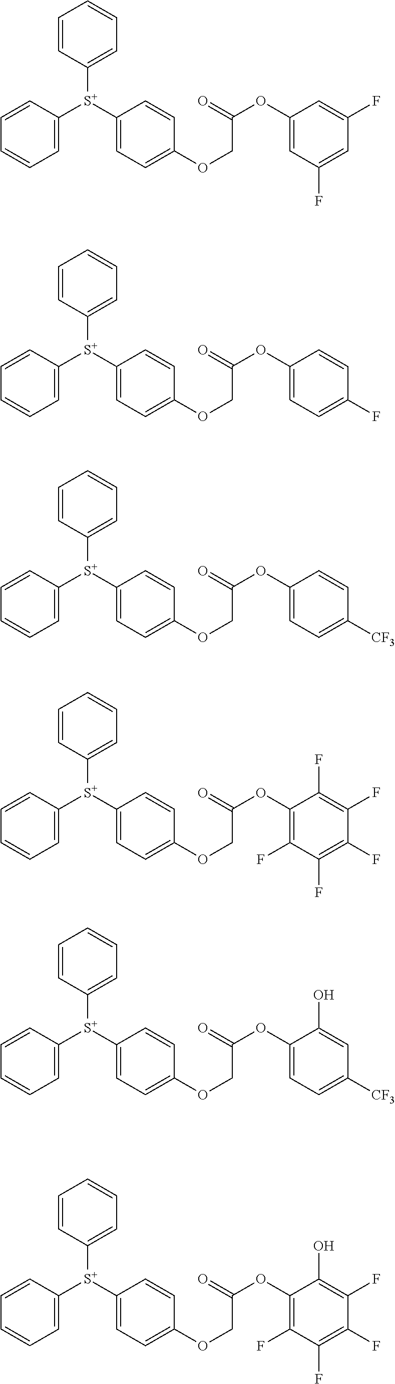 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F]

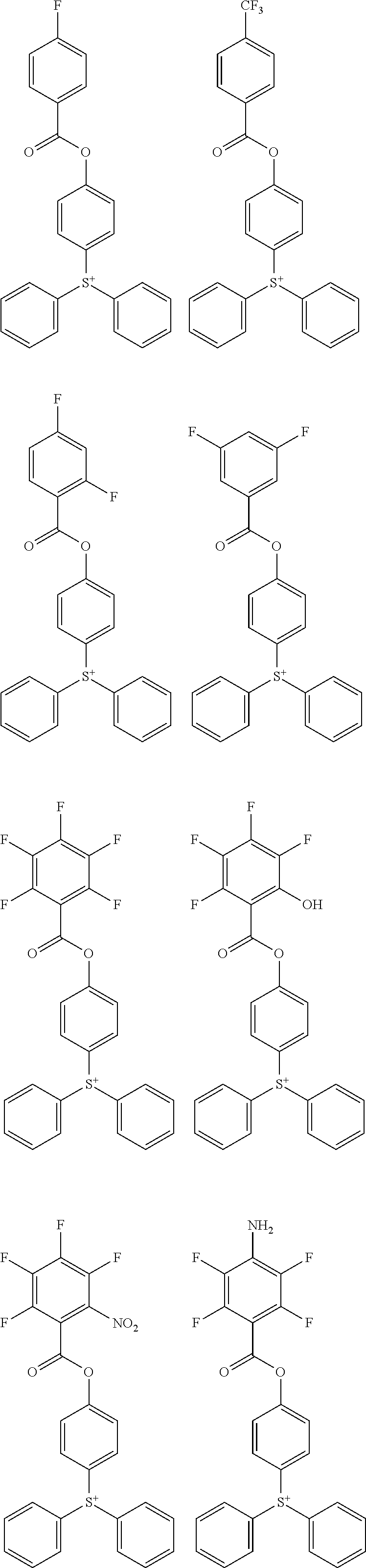

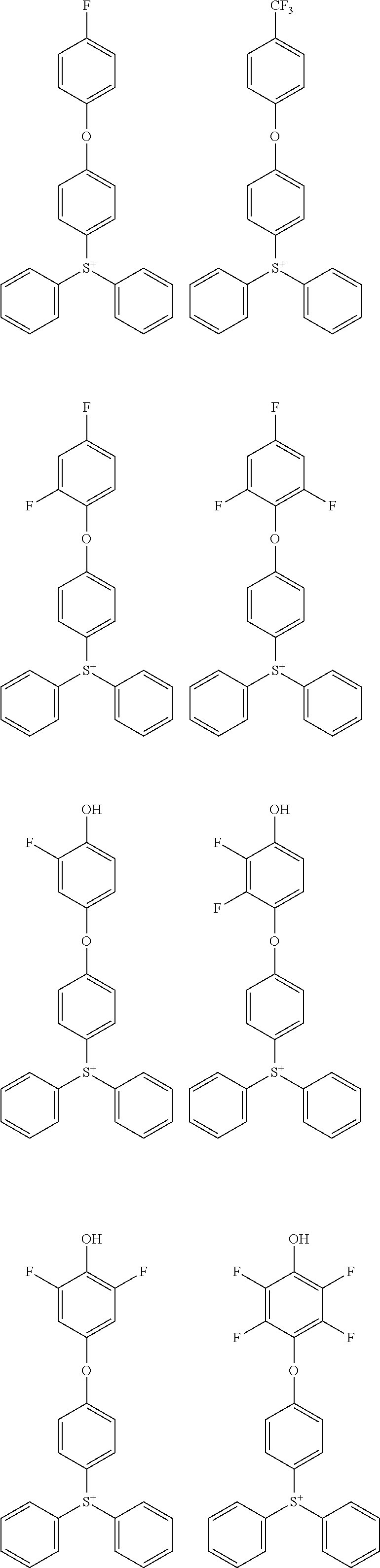

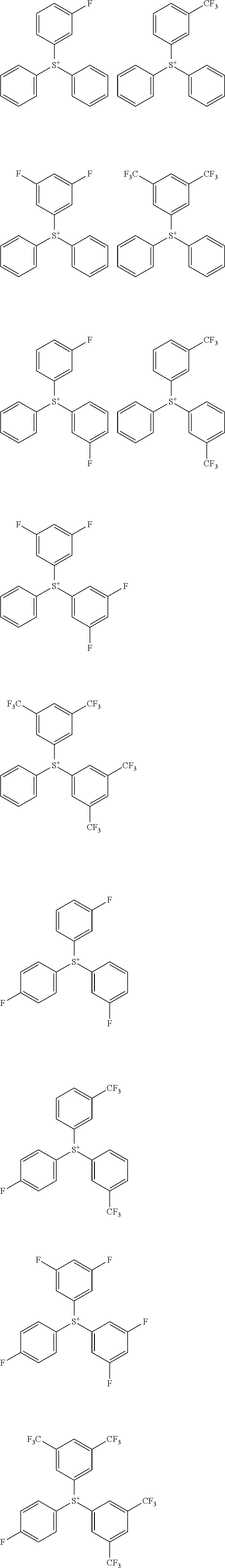

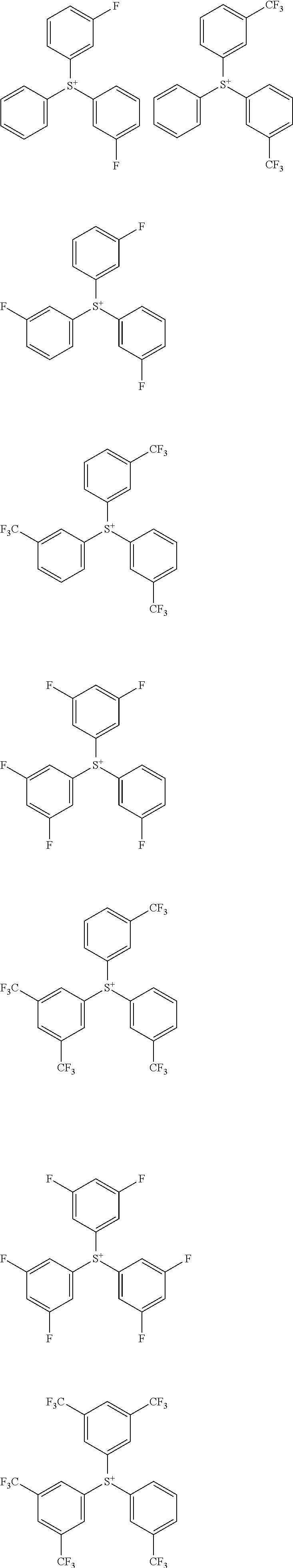

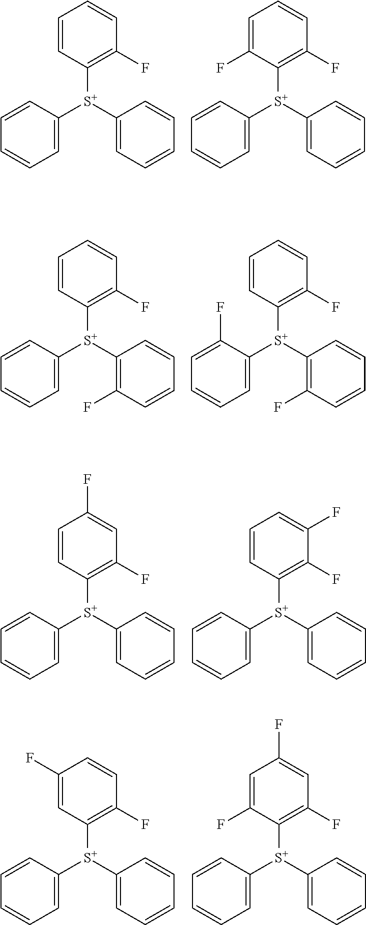

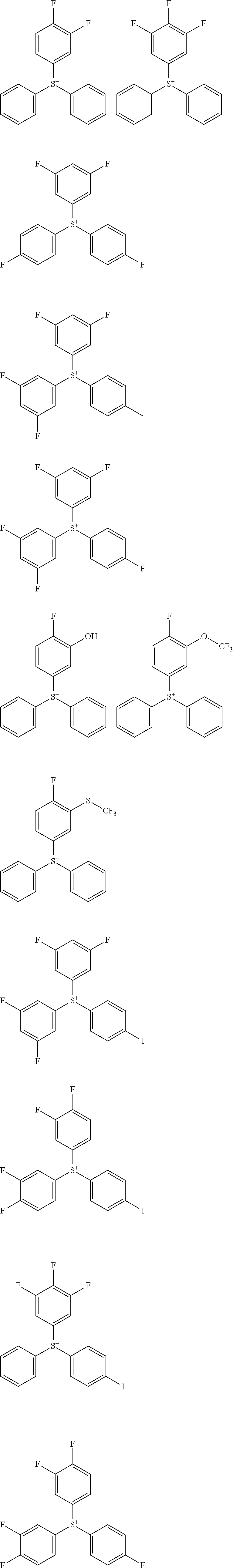

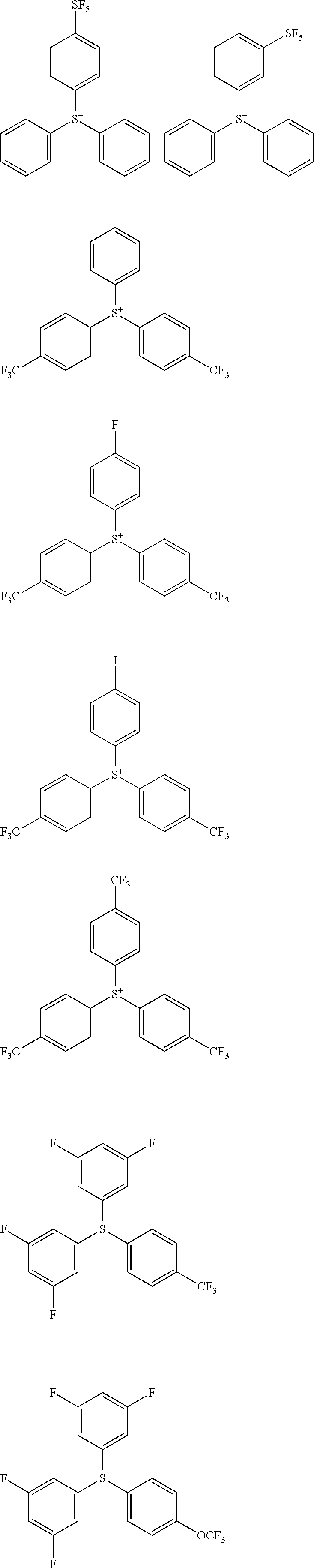

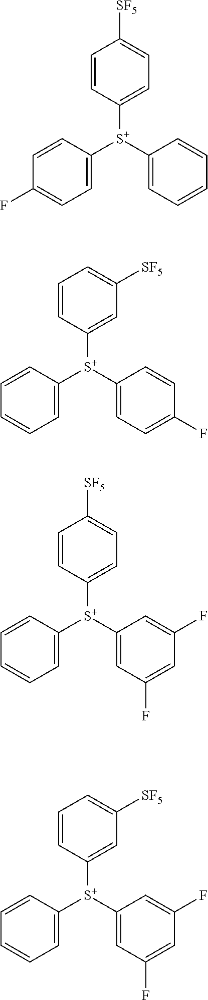

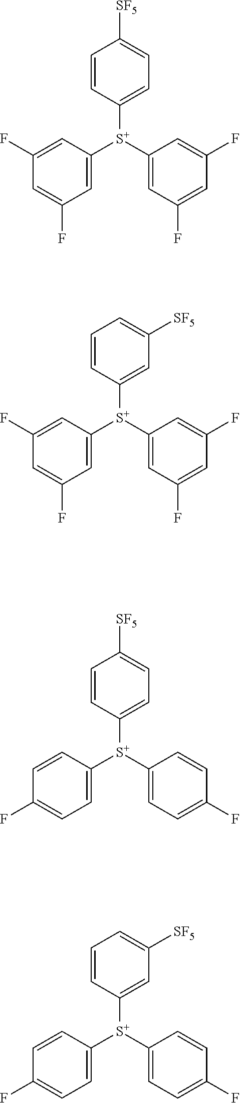

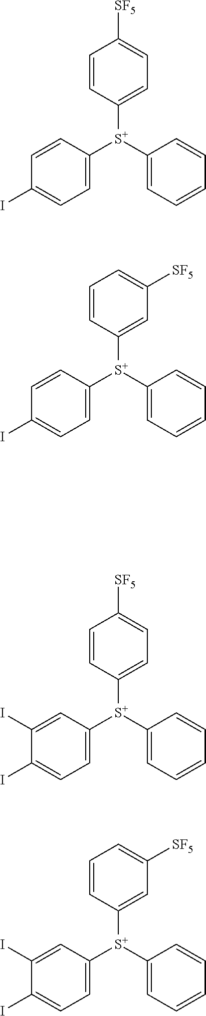

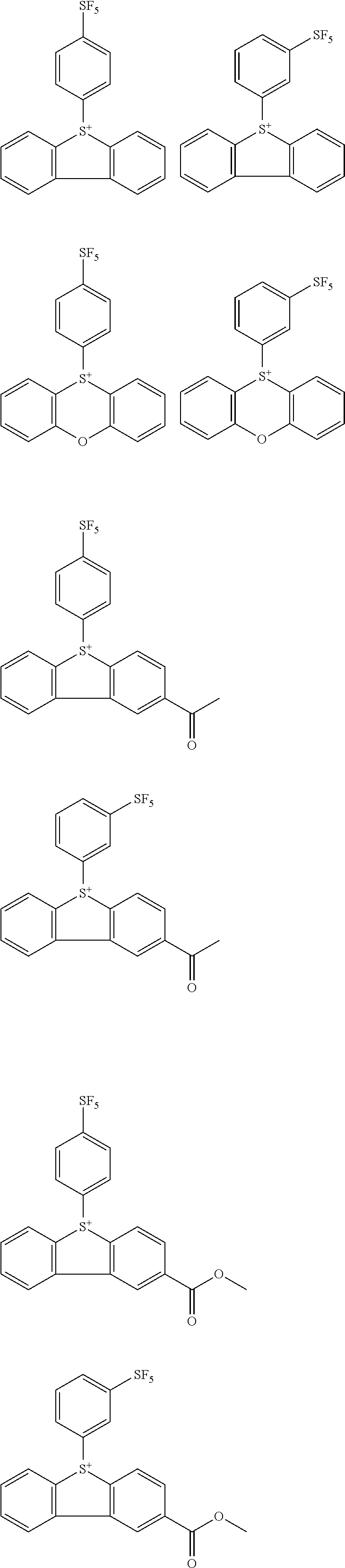

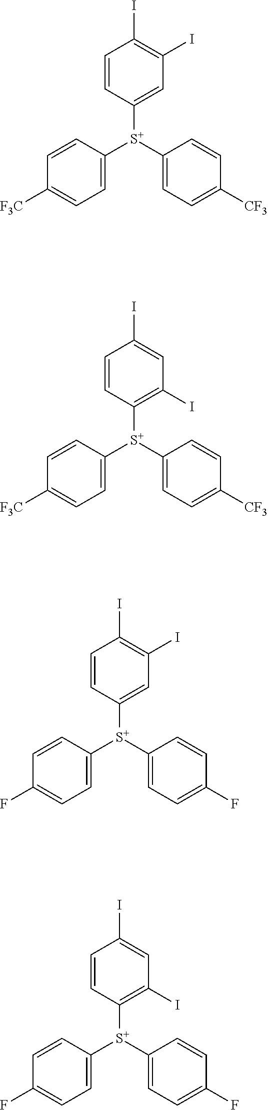 [SMILES: FC(F)(F)c1ccc([S+](c2ccc(C(F)(F)F)cc2)c2ccc(I)c(I)c2)cc1 ; FC(F)(F)c1ccc([S+](c2ccc(C(F)(F)F)cc2)c2ccc(I)cc2I)cc1 ; Fc1ccc([S+](c2ccc(F)cc2)c2ccc(I)c(I)c2)cc1 ; Fc1ccc([S+](c2ccc(F)cc2)c2ccc(I)cc2I)cc1]

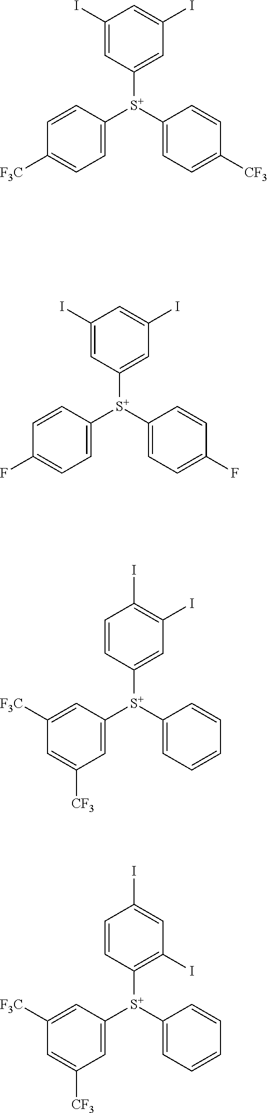 [SMILES: FC(F)(F)c1ccc([S+](c2ccc(C(F)(F)F)cc2)c2cc(I)cc(I)c2)cc1 ; Fc1ccc([S+](c2ccc(F)cc2)c2cc(I)cc(I)c2)cc1 ; FC(F)(F)c1cc([S+](c2ccccc2)c2ccc(I)c(I)c2)cc(C(F)(F)F)c1 ; FC(F)(F)c1cc([S+](c2ccccc2)c2ccc(I)cc2I)cc(C(F)(F)F)c1]

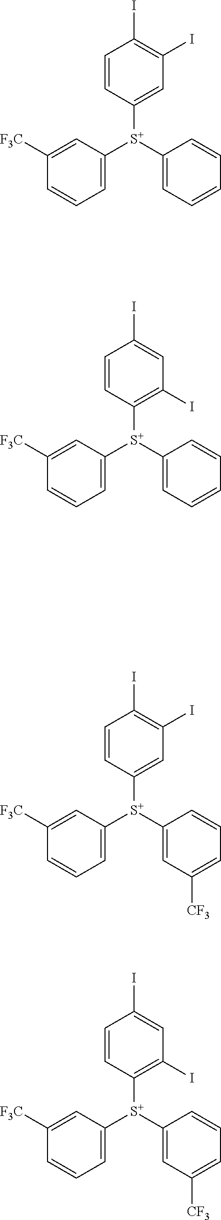 [SMILES: FC(F)(F)c1cccc([S+](c2ccccc2)c2ccc(I)c(I)c2)c1 ; FC(F)(F)c1cccc([S+](c2ccccc2)c2ccc(I)cc2I)c1 ; FC(F)(F)c1cccc([S+](c2cccc(C(F)(F)F)c2)c2ccc(I)c(I)c2)c1 ; FC(F)(F)c1cccc([S+](c2cccc(C(F)(F)F)c2)c2ccc(I)cc2I)c1]

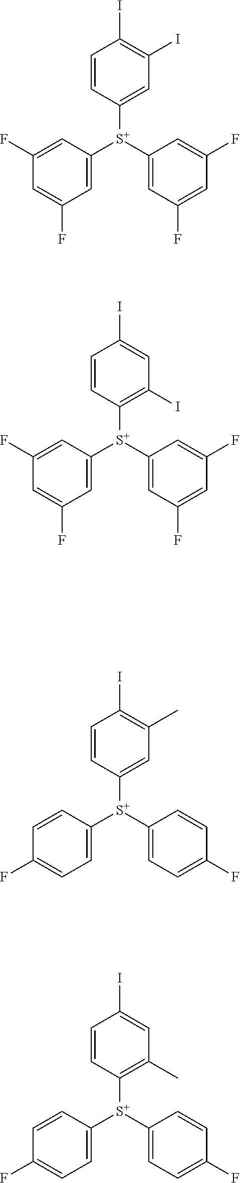 [SMILES: Fc1cc(F)cc([S+](c2cc(F)cc(F)c2)c2ccc(I)c(I)c2)c1 ; Fc1cc(F)cc([S+](c2cc(F)cc(F)c2)c2ccc(I)cc2I)c1 ; Cc1cc([S+](c2ccc(F)cc2)c2ccc(F)cc2)ccc1I ; Cc1cc(I)ccc1[S+](c1ccc(F)cc1)c1ccc(F)cc1]

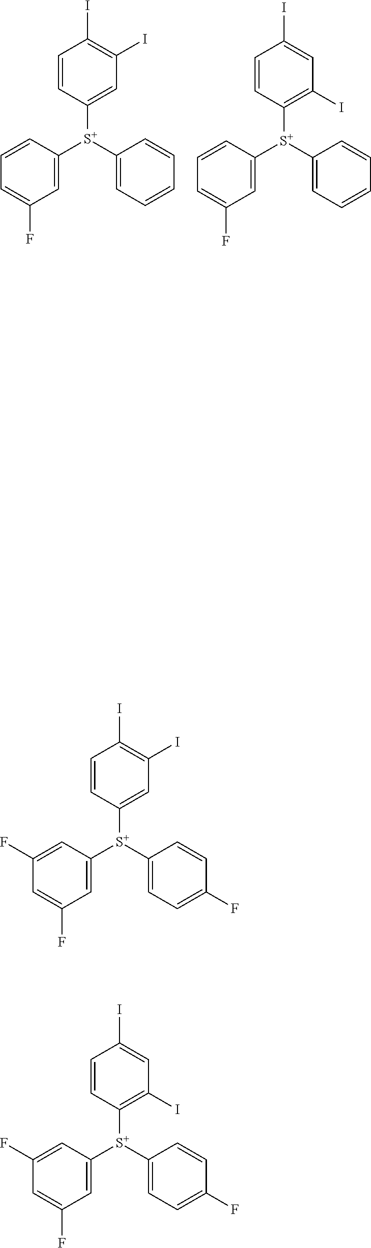 [SMILES: Fc1cccc([S+](c2ccccc2)c2ccc(I)c(I)c2)c1 ; Fc1cccc([S+](c2ccccc2)c2ccc(I)cc2I)c1 ; Fc1ccc([S+](c2cc(F)cc(F)c2)c2ccc(I)c(I)c2)cc1 ; Fc1ccc([S+](c2cc(F)cc(F)c2)c2ccc(I)cc2I)cc1]

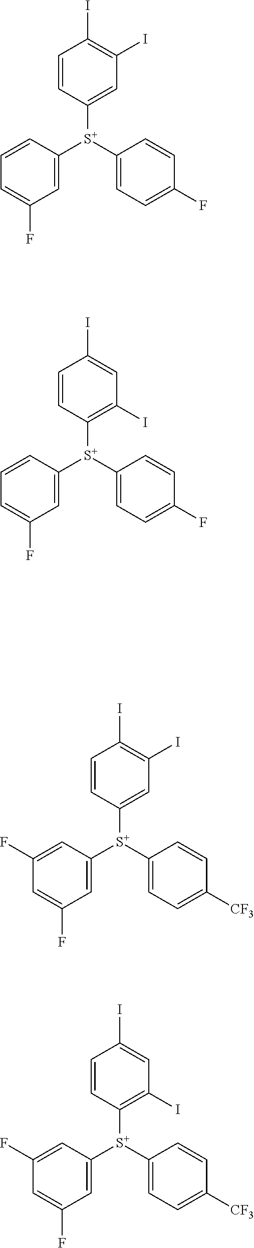 [SMILES: Fc1ccc([S+](c2cccc(F)c2)c2ccc(I)c(I)c2)cc1 ; Fc1ccc([S+](c2cccc(F)c2)c2ccc(I)cc2I)cc1 ; Fc1cc(F)cc([S+](c2ccc(C(F)(F)F)cc2)c2ccc(I)c(I)c2)c1 ; Fc1cc(F)cc([S+](c2ccc(C(F)(F)F)cc2)c2ccc(I)cc2I)c1]

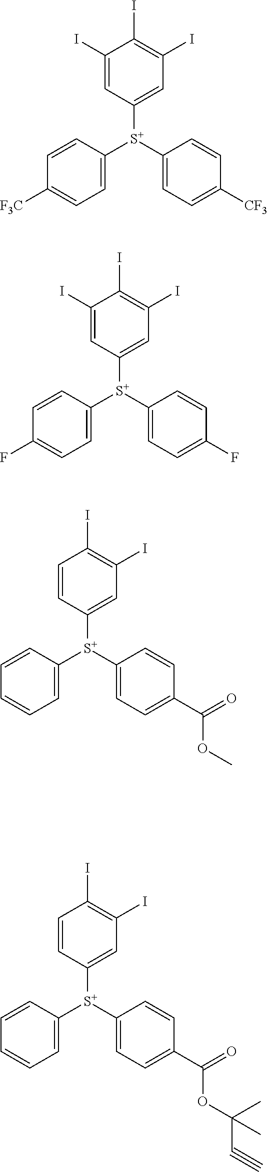 [SMILES: FC(F)(F)c1ccc([S+](c2ccc(C(F)(F)F)cc2)c2cc(I)c(I)c(I)c2)cc1 ; Fc1ccc([S+](c2ccc(F)cc2)c2cc(I)c(I)c(I)c2)cc1 ; COC(=O)c1ccc([S+](c2ccccc2)c2ccc(I)c(I)c2)cc1 ; C#CC(C)(C)OC(=O)c1ccc([S+](c2ccccc2)c2ccc(I)c(I)c2)cc1]

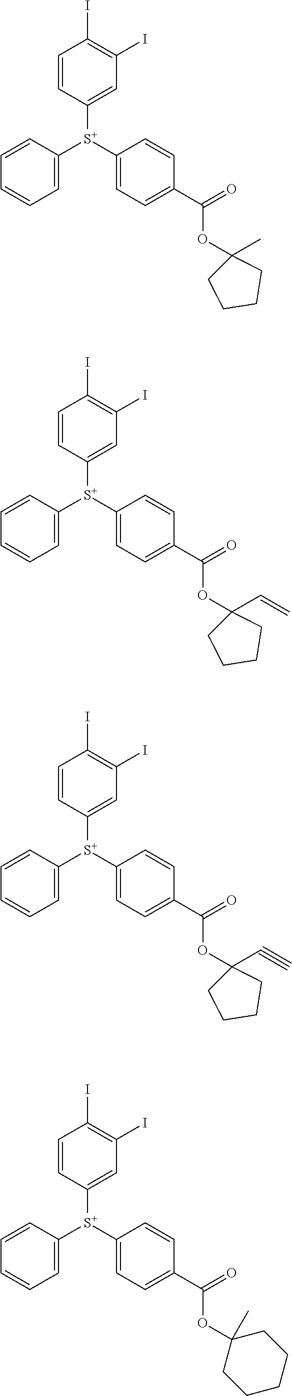 [SMILES: CC1(OC(=O)c2ccc([S+](c3ccccc3)c3ccc(I)c(I)c3)cc2)CCCC1 ; C=CC1(OC(=O)c2ccc([S+](c3ccccc3)c3ccc(I)c(I)c3)cc2)CCCC1 ; C#CC1(OC(=O)c2ccc([S+](c3ccccc3)c3ccc(I)c(I)c3)cc2)CCCC1 ; CC1(OC(=O)c2ccc([S+](c3ccccc3)c3ccc(I)c(I)c3)cc2)CCCCC1]

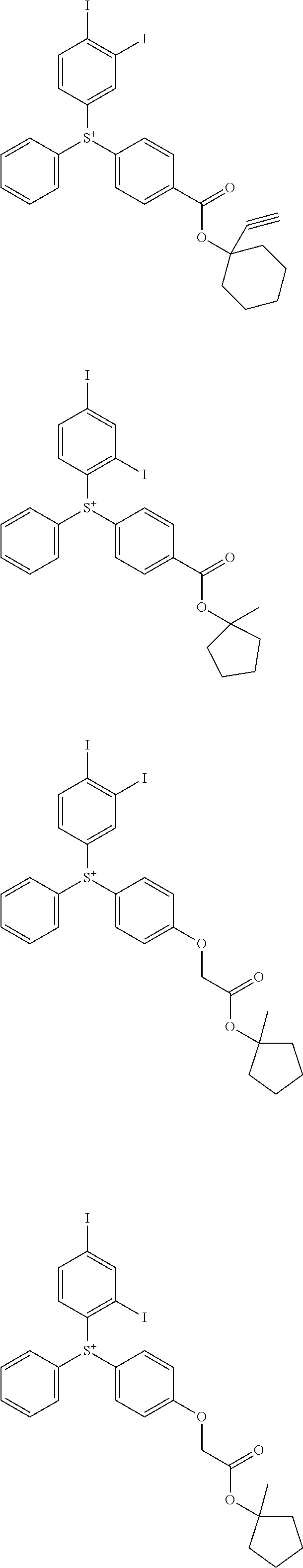 [SMILES: C#CC1(OC(=O)c2ccc([S+](c3ccccc3)c3ccc(I)c(I)c3)cc2)CCCCC1 ; CC1(OC(=O)c2ccc([S+](c3ccccc3)c3ccc(I)cc3I)cc2)CCCC1 ; CC1(OC(=O)COc2ccc([S+](c3ccccc3)c3ccc(I)c(I)c3)cc2)CCCC1 ; CC1(OC(=O)COc2ccc([S+](c3ccccc3)c3ccc(I)cc3I)cc2)CCCC1]

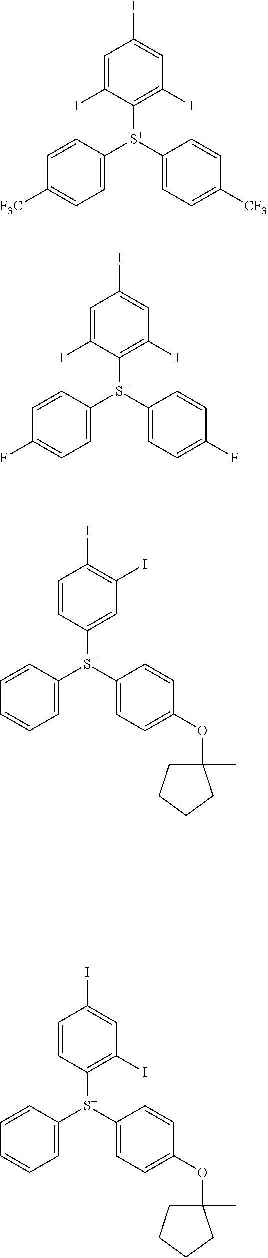 [SMILES: FC(F)(F)c1ccc([S+](c2ccc(C(F)(F)F)cc2)c2c(I)cc(I)cc2I)cc1 ; Fc1ccc([S+](c2ccc(F)cc2)c2c(I)cc(I)cc2I)cc1 ; CC1(Oc2ccc([S+](c3ccccc3)c3ccc(I)c(I)c3)cc2)CCCC1 ; CC1(Oc2ccc([S+](c3ccccc3)c3ccc(I)cc3I)cc2)CCCC1]

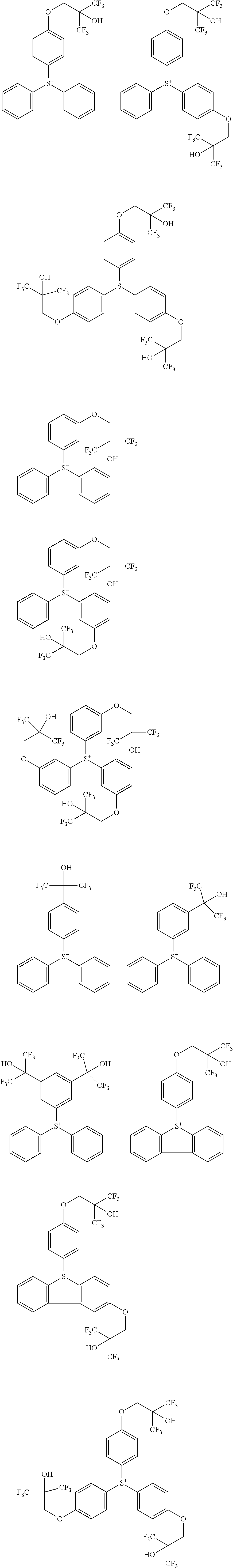

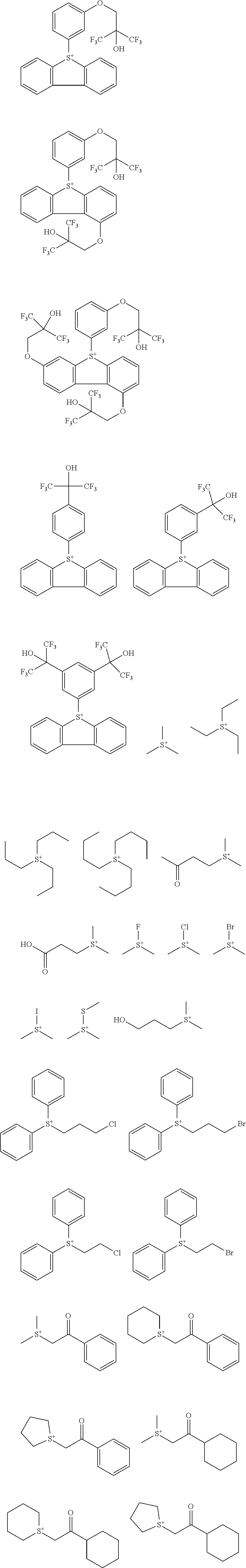

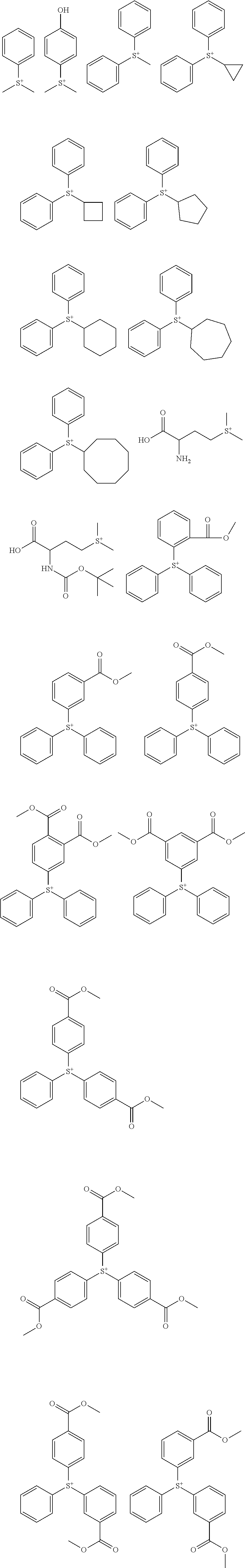

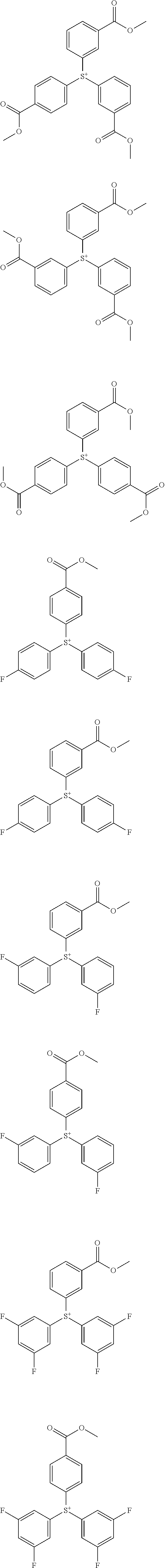

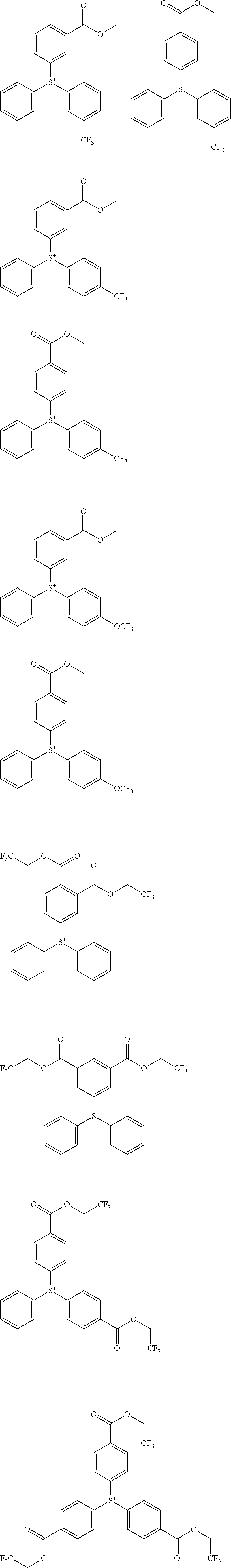

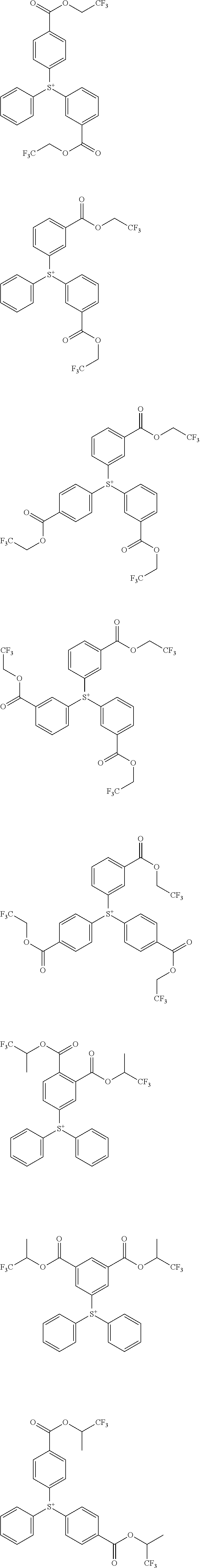

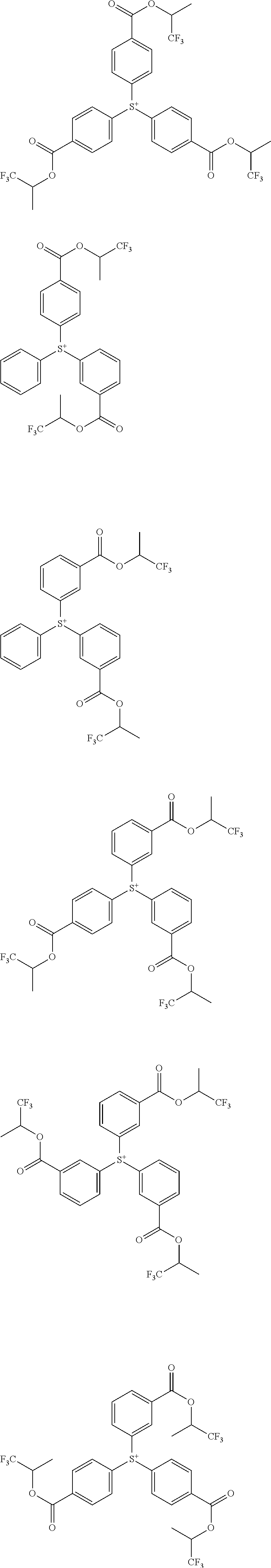

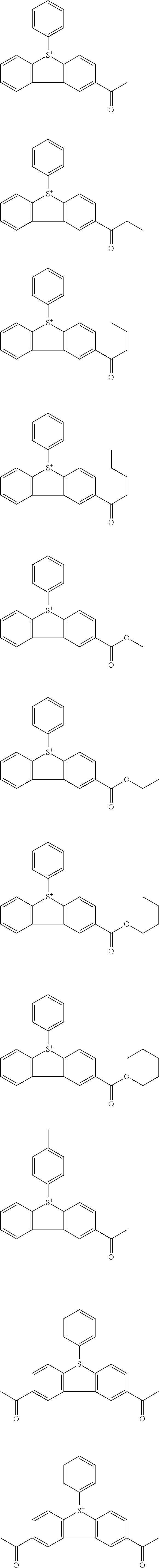

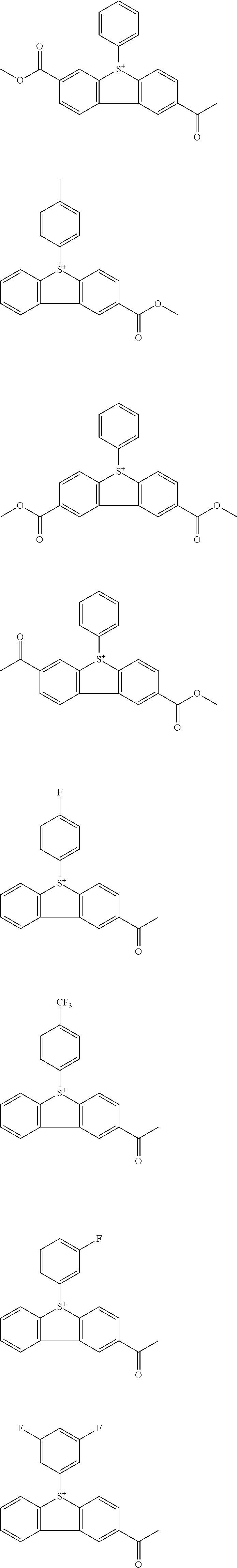

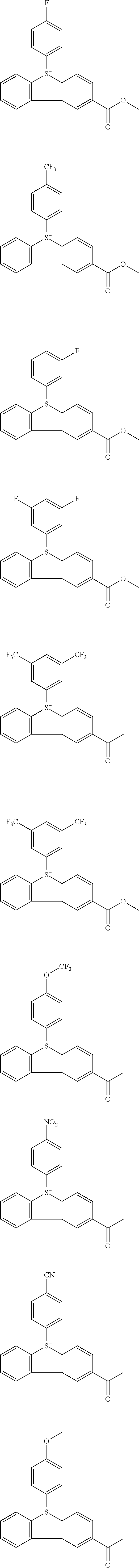

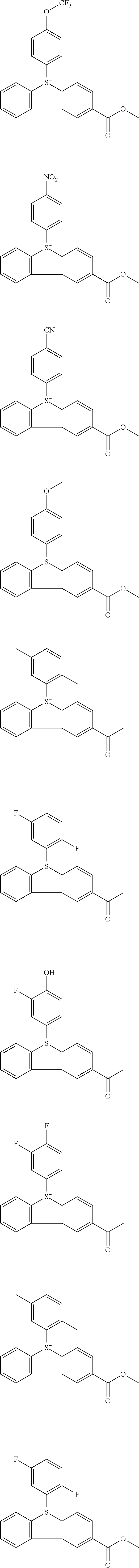

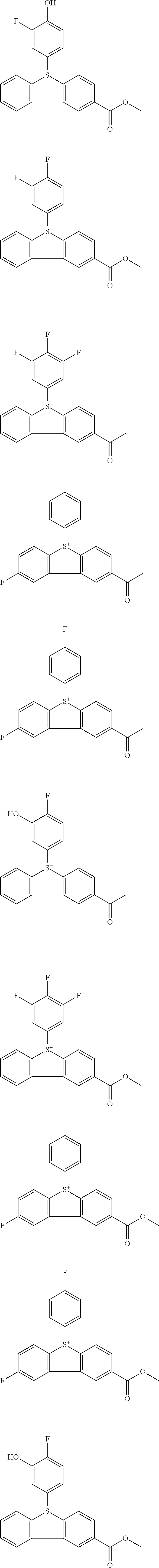

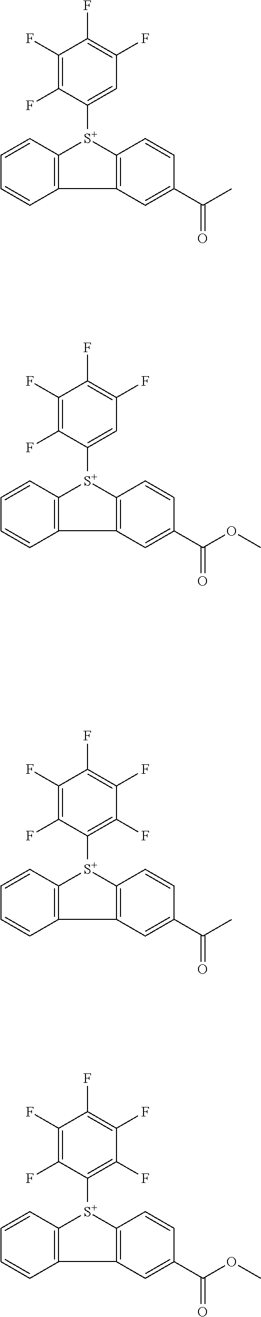 [SMILES: CC(=O)c1ccc2c(c1)c1ccccc1[s+]2-c1cc(F)c(F)c(F)c1F ; COC(=O)c1ccc2c(c1)c1ccccc1[s+]2-c1cc(F)c(F)c(F)c1F ; CC(=O)c1ccc2c(c1)c1ccccc1[s+]2-c1c(F)c(F)c(F)c(F)c1F ; COC(=O)c1ccc2c(c1)c1ccccc1[s+]2-c1c(F)c(F)c(F)c(F)c1F]

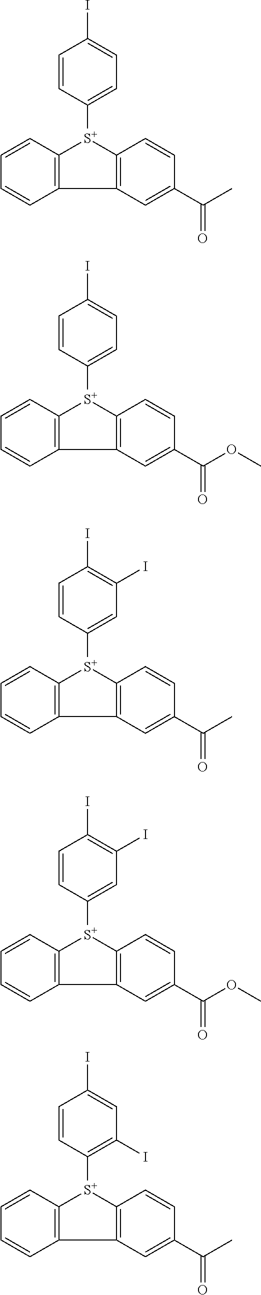 [SMILES: CC(=O)c1ccc2c(c1)c1ccccc1[s+]2-c1ccc(I)cc1 ; COC(=O)c1ccc2c(c1)c1ccccc1[s+]2-c1ccc(I)cc1 ; CC(=O)c1ccc2c(c1)c1ccccc1[s+]2-c1ccc(I)c(I)c1 ; COC(=O)c1ccc2c(c1)c1ccccc1[s+]2-c1ccc(I)c(I)c1 ; CC(=O)c1ccc2c(c1)c1ccccc1[s+]2-c1ccc(I)cc1I]

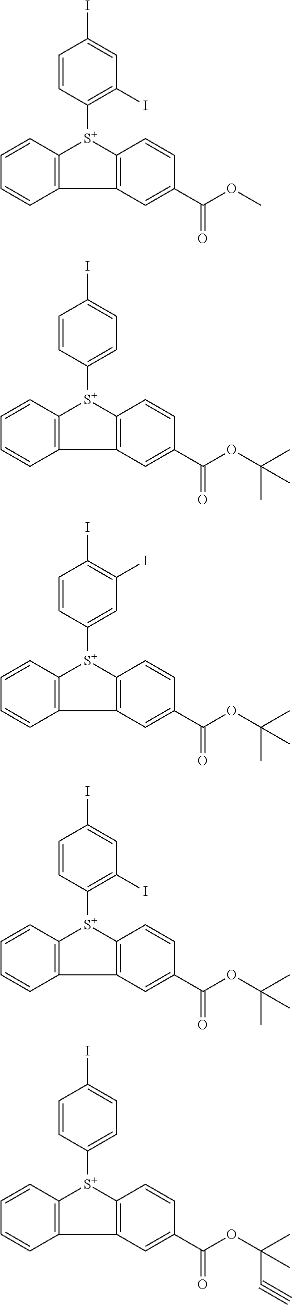 [SMILES: COC(=O)c1ccc2c(c1)c1ccccc1[s+]2-c1ccc(I)cc1I ; CC(C)(C)OC(=O)c1ccc2c(c1)c1ccccc1[s+]2-c1ccc(I)cc1 ; CC(C)(C)OC(=O)c1ccc2c(c1)c1ccccc1[s+]2-c1ccc(I)c(I)c1 ; CC(C)(C)OC(=O)c1ccc2c(c1)c1ccccc1[s+]2-c1ccc(I)cc1I ; C#CC(C)(C)OC(=O)c1ccc2c(c1)c1ccccc1[s+]2-c1ccc(I)cc1]

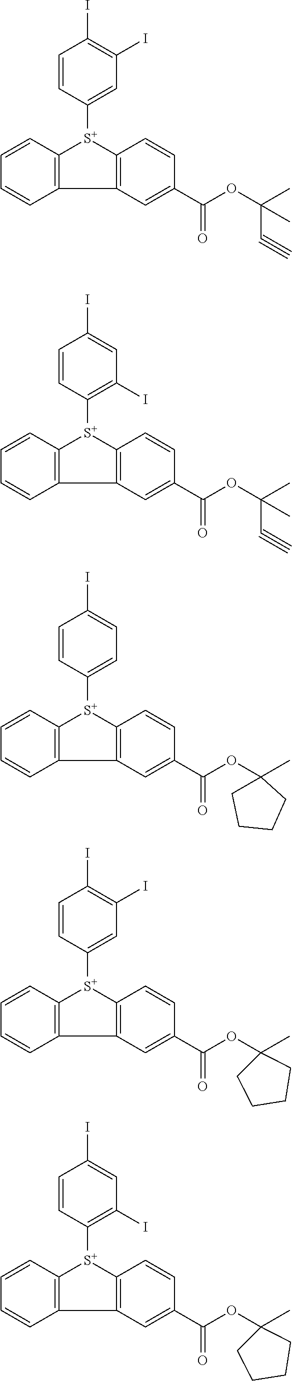 [SMILES: C#CC(C)(C)OC(=O)c1ccc2c(c1)c1ccccc1[s+]2-c1ccc(I)c(I)c1 ; C#CC(C)(C)OC(=O)c1ccc2c(c1)c1ccccc1[s+]2-c1ccc(I)cc1I ; CC1(OC(=O)c2ccc3c(c2)c2ccccc2[s+]3-c2ccc(I)cc2)CCCC1 ; CC1(OC(=O)c2ccc3c(c2)c2ccccc2[s+]3-c2ccc(I)c(I)c2)CCCC1 ; CC1(OC(=O)c2ccc3c(c2)c2ccccc2[s+]3-c2ccc(I)cc2I)CCCC1]

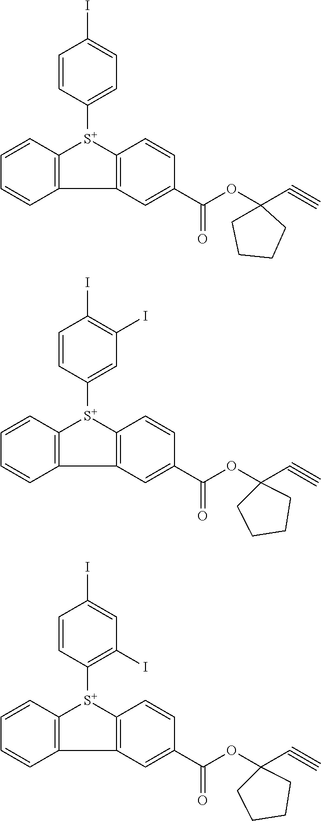 [SMILES: C#CC1(OC(=O)c2ccc3c(c2)c2ccccc2[s+]3-c2ccc(I)cc2)CCCC1 ; C#CC1(OC(=O)c2ccc3c(c2)c2ccccc2[s+]3-c2ccc(I)c(I)c2)CCCC1 ; C#CC1(OC(=O)c2ccc3c(c2)c2ccccc2[s+]3-c2ccc(I)cc2I)CCCC1]

Specific examples of the iodonium cation represented by the formula (Z-2) include ones shown below, but are not limited thereto.

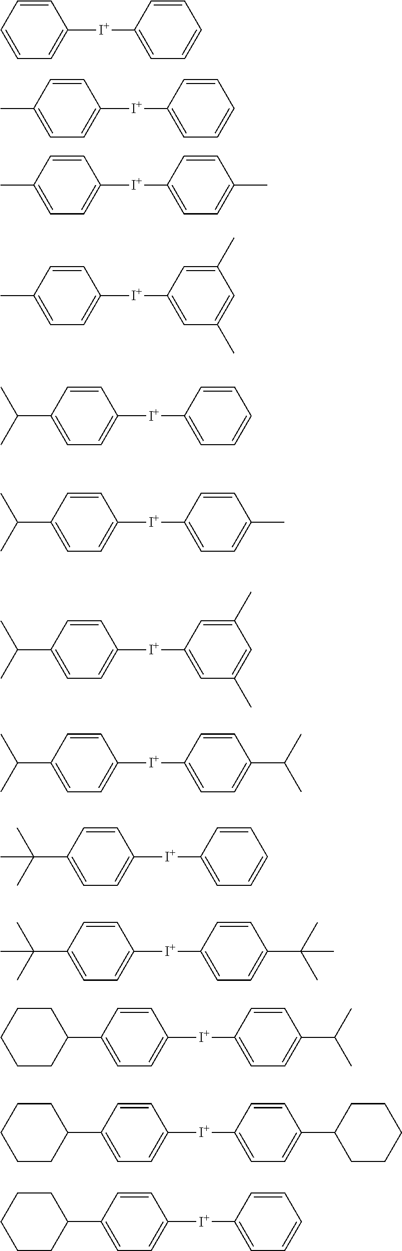

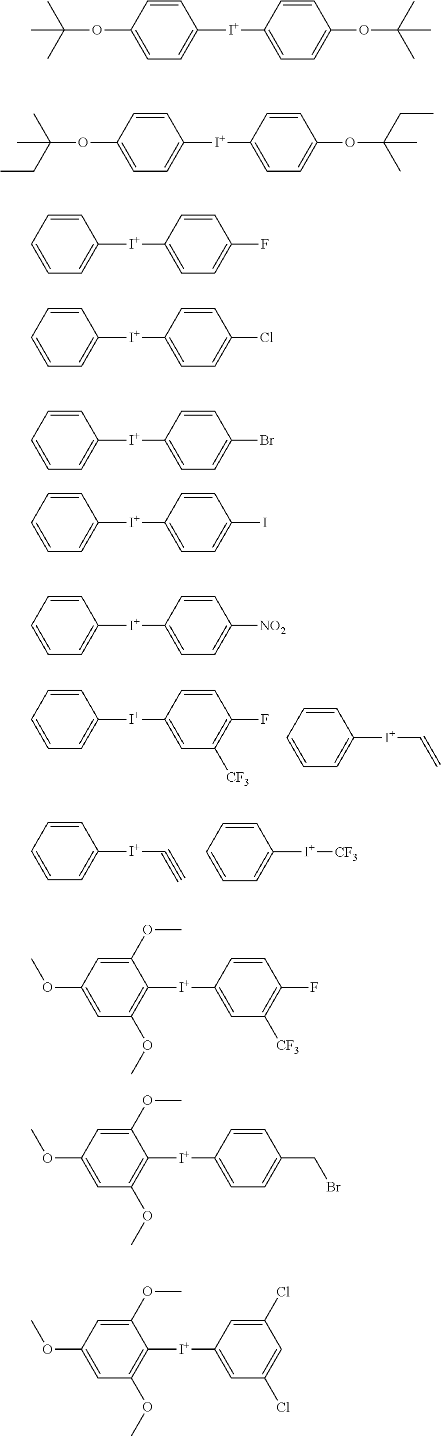

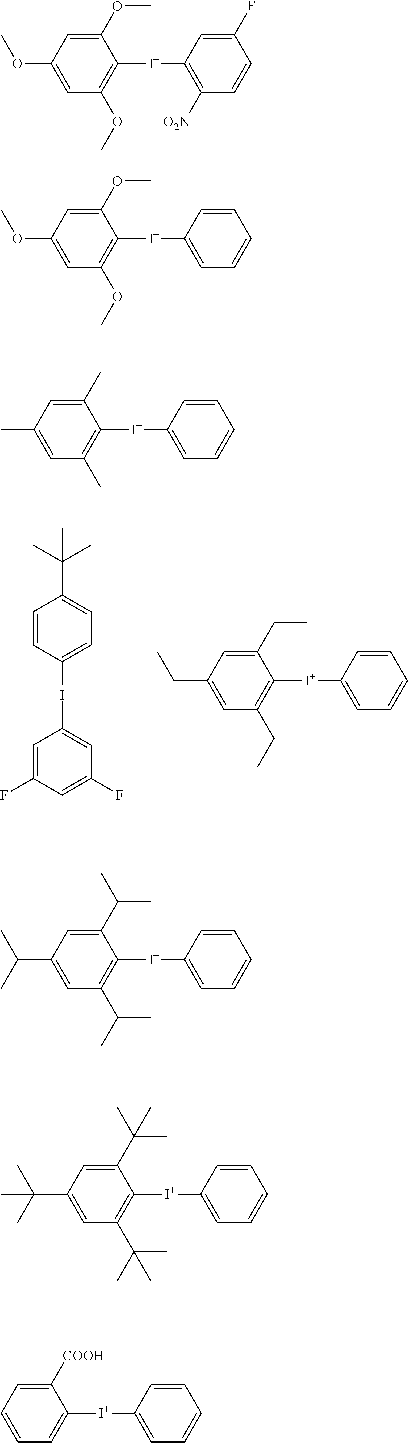

Specific examples of the inventive onium salt monomer include any combination of the above-described anions and the cations.

The inventive onium salt monomer can be synthesized, for example, by the same method as that of the polymerizable anion-containing sulfonium salt disclosed in JP5201363B2, but the method is not limited thereto.

### [Monomeric Photo-Acid Generator]

The present invention also provides a monomeric photo-acid generator including the above-described onium salt monomer. The inventive onium salt monomer can be used suitably as a monomeric photo-acid generator.

### [Polymer]

The inventive polymer contains a repeating unit derived from the onium salt monomer (monomeric photo-acid generator) represented by the formula (A) (hereinafter, also referred to as repeating unit-a).

The inventive polymer is a polymer-bound photo-acid generator that functions as a photo-acid generator in a chemically-amplified resist composition, and also functions as a base polymer. Structural characteristics of the inventive polymer include the fact that the polymer includes a repeating unit derived from a sulfonium salt or an iodonium salt each including an iodobenzenesulfonate anion containing an aromatic ring. Iodine atoms very greatly absorb EUV having a wavelength of 13.5 nm, and therefore, secondary electrons are generated during exposure. Decomposition is promoted by the energy of the secondary electrons being transferred to the acid generator, and thus, high sensitivity is achieved. The iodobenzenesulfonate anion can enhance the acidity of the sulfonic acid, and in particular, acidity can be further enhanced by iodine atoms being substituents in the meta positions or the para position of the carbon atom bonded to the sulfonic acid. That is, there is a characteristic that out of the two carbon atoms present in the ortho positions of the sulfonic acid, one is not bonded to an iodine atom, and therefore, the sulfonic acid is not affected by the sterically bulky electron clouds of iodine atoms, and favorable sensitivity and favorable resolution can be exhibited. It is possible to form a pattern that is excellent in the LWR of a line pattern and the CDU of a hole pattern and that is resistant to pattern collapse, and therefore, the inventive polymer is suitable particularly as a material for a chemically-amplified positive resist composition.

In the case of a positive resist material, the inventive polymer can include a repeating unit containing an acid-labile group. As the repeating unit containing an acid-labile group, a repeating unit represented by the following formula (b1) (hereinafter, also referred to as repeating unit-b1) or a repeating unit represented by the formula (b2) (hereinafter, also referred to as repeating unit-b2) is preferable.

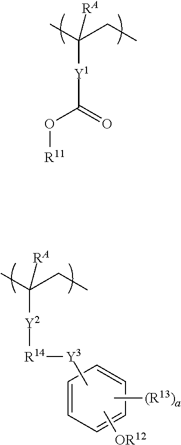

In the formulae (b1) and (b2), each R^{A} independently represents a hydrogen atom or a methyl group;
Y¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally having at least one selected from a hydroxy group, a halogen atom, a saturated hydrocarbyloxy group having 1 to 8 carbon atoms, and a saturated hydrocarbylcarbonyloxy group having 2 to 8 carbon atoms;
Y² represents a single bond or an ester bond;
Y³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.

Specific examples of a monomer to give the repeating unit-b1 include ones shown below, but are not limited thereto. Note that, in the following formulae, R^{A} and R¹¹ are as defined above.

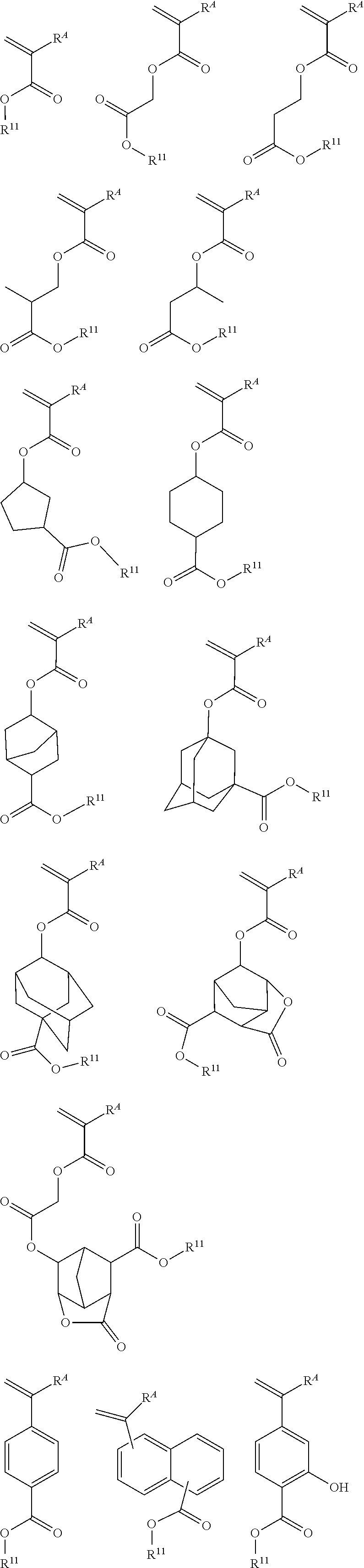

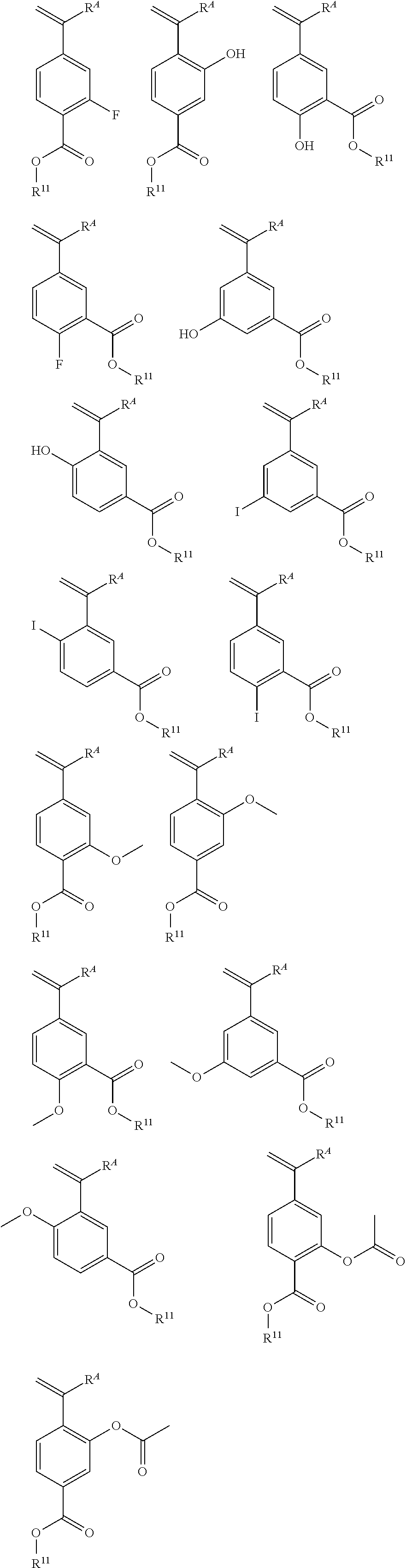

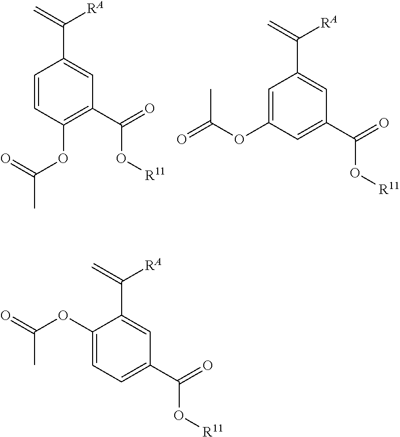

Specific examples of a monomer to give the repeating unit-b2 include ones shown below, but are not limited thereto. Note that, in the following formulae, R^{A} and R¹² are as defined above.

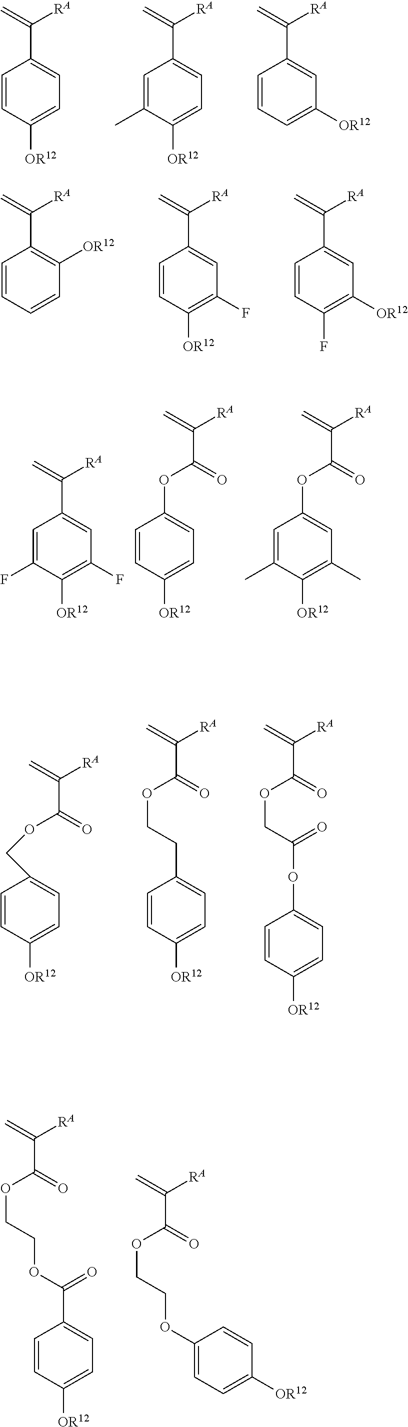

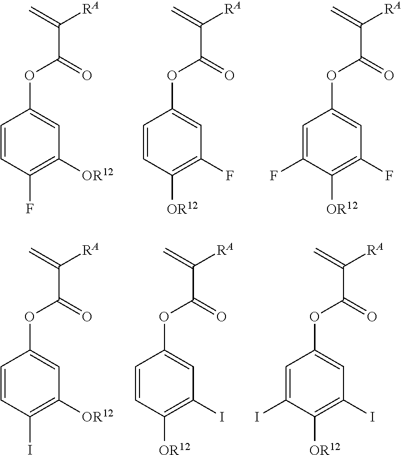

Various acid-labile groups can be selected as the acid-labile groups represented by R¹¹ or R¹². Examples thereof include ones represented by the following formulae (AL-1) to (AL-3).

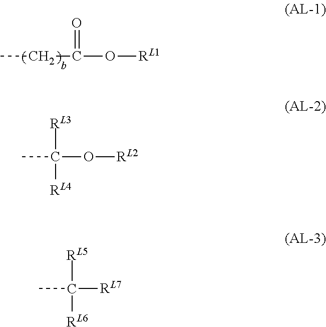

In the formulae, a broken line represents an attachment point.

In the formula (AL-1), “b” represents an integer of 0 to 6. R^{L1} represents: a tertiary hydrocarbyl group having 4 to 20, preferably 4 to 15 carbon atoms; a trihydrocarbylsilyl group in which hydrocarbyl groups are each a saturated hydrocarbyl group having 1 to 6 carbon atoms; a saturated hydrocarbyl group having 4 to 20 carbon atoms and containing a carbonyl group, an ether bond, or an ester bond; or a group represented by the formula (AL-3). Note that a tertiary hydrocarbyl group means a group obtained by a hydrogen atom being removed from a tertiary carbon atom of a hydrocarbon.

The tertiary hydrocarbyl group represented by R^{L}1 may be saturated or unsaturated, and may be branched or cyclic. Specific examples thereof include a tert-butyl group, a tert-pentyl group, a 1,1-diethylpropyl group, a 1-ethylcyclopentyl group, a 1-butylcyclopentyl group, a 1-ethylcyclohexyl group, a 1-butylcyclohexyl group, a 1-ethyl-2-cyclopentenyl group, a 1-ethyl-2-cyclohexenyl group, a 2-methyl-2-adamantyl group, etc. Examples of the trihydrocarbylsilyl group include a trimethylsilyl group, a triethylsilyl group, a dimethyl-tert-butylsilyl group, etc. The saturated hydrocarbyl group containing a carbonyl group, an ether bond, or an ester bond may be linear, branched, or cyclic, and is preferably cyclic. Specific examples thereof include a 3-oxocyclohexyl group, a 4-methyl-2-oxooxan-4-yl group, a 5-methyl-2-oxooxolan-5-yl group, a 2-tetrahydropyranyl group, a 2-tetrahydrofuranyl group, etc.

Examples of the acid-labile group represented by the formula (AL-1) include a tert-butoxycarbonyl group, a tert-butoxycarbonylmethyl group, a tert-pentyloxycarbonyl group, a tert-pentyloxycarbonylmethyl group, a 1,1-diethylpropyloxycarbonyl group, a 1,1-diethylpropyloxycarbonylmethyl group, a 1-ethylcyclopentyloxycarbonyl group, a 1-ethylcyclopentyloxycarbonylmethyl group, a 1-ethyl-2-cyclopentenyloxycarbonyl group, a 1-ethyl-2-cyclopentenyloxycarbonylmethyl group, a 1-ethoxyethoxycarbonylmethyl group, a 2-tetrahydropyranyloxycarbonylmethyl group, a 2-tetrahydrofuranyloxycarbonylmethyl group, etc.

Examples of the acid-labile group represented by the formula (AL-1) further include groups represented by the following formulae (AL-1)-1 to (AL-1)-10.

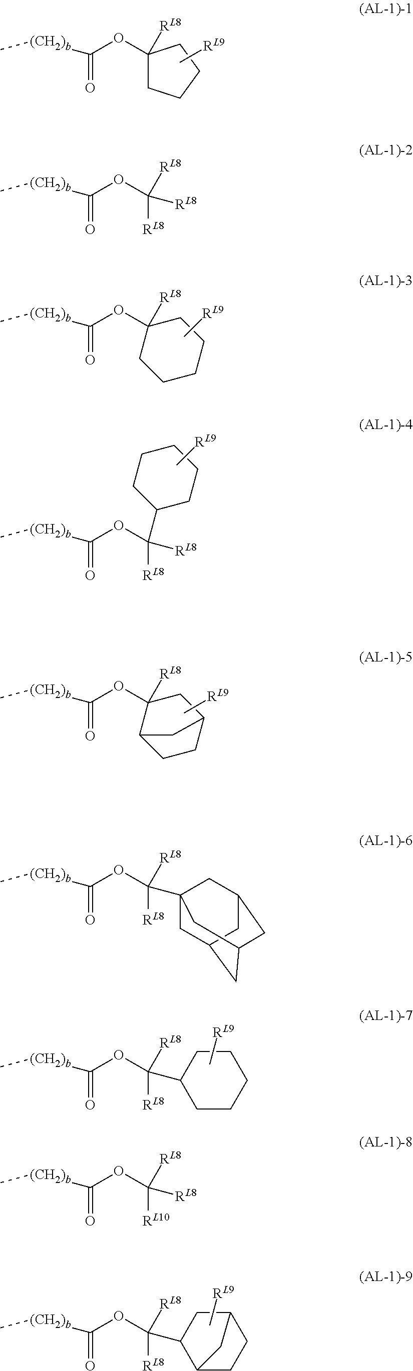

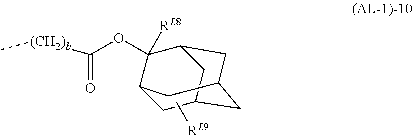

In the formulae, a broken line represents an attachment point.

In the formulae (AL-1)-1 to (AL-1)-10, “b” is as defined above. Each R^{L8} independently represents a saturated hydrocarbyl group having 1 to 10 carbon atoms or an aryl group having 6 to 20 carbon atoms. R^{L9} represents a hydrogen atom or a saturated hydrocarbyl group having 1 to 10 carbon atoms. R^{L10} represents a saturated hydrocarbyl group having 2 to 10 carbon atoms or an aryl group having 6 to 20 carbon atoms. The saturated hydrocarbyl groups may be linear, branched, or cyclic.

In the formula (AL-2), R^{L2} and R^{L3} each independently represent a hydrogen atom or a saturated hydrocarbyl group having 1 to 18, preferably 1 to 10 carbon atoms. The saturated hydrocarbyl group may be linear, branched, or cyclic. Specific examples thereof include a methyl group, an ethyl group, an n-propyl group, an isopropyl group, an n-butyl group, an isobutyl group, a sec-butyl group, a tert-butyl group, a cyclopentyl group, a cyclohexyl group, a 2-ethylhexyl group, an n-octyl group, etc.

In the formula (AL-2), R^{L4} represents a hydrocarbyl group having 1 to 18, preferably 1 to 10 carbon atoms, and optionally contains a heteroatom. The hydrocarbyl group may be saturated or unsaturated, and may be linear, branched, or cyclic. Examples of the hydrocarbyl group include saturated hydrocarbyl groups having 1 to 18 carbon atoms, etc., and part of the hydrogen atoms thereof may be substituted with a hydroxy group, an alkoxy group, an oxo group, an amino group, an alkylamino group, etc. Examples of such substituted saturated hydrocarbyl groups include ones shown below, etc.

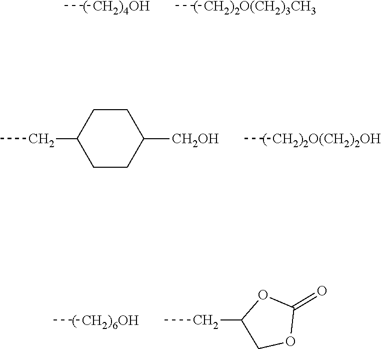

In the formulae, a broken line represents an attachment point.
R^{L2} and R^{L3}, R^{L2} and R^{L4}, or R^{L3} and R^{L4} optionally bond with each other to form a ring together with the carbon atom bonded therewith, or together with the carbon atom and the oxygen atom. In this case, R^{L2} and R^{L3}, R^{L2} and R^{L4}, or R^{L3} and R^{L4}, involved in the ring formation, each independently represent an alkanediyl group having 1 to 18, preferably 1 to 10 carbon atoms. The number of carbon atoms in the ring obtained by bonding these is preferably 3 to 10, more preferably 4 to 10.

Examples of the linear or branched ones among the acid-labile groups shown by the formula (AL-2) include ones represented by the following formulae (AL-2)-1 to (AL-2)-69, but are not limited thereto. Note that, in the following formulae, a broken line represents an attachment point.

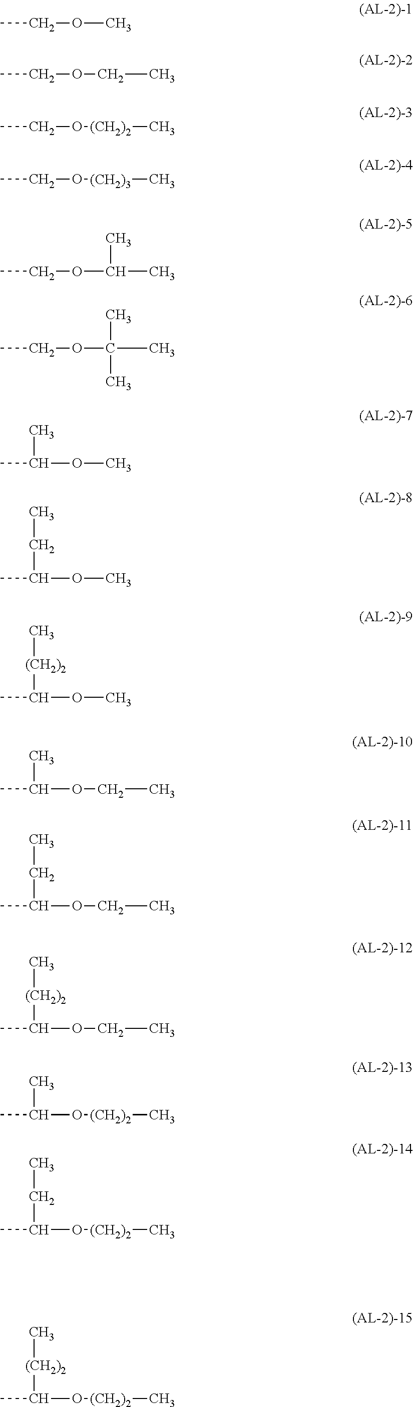

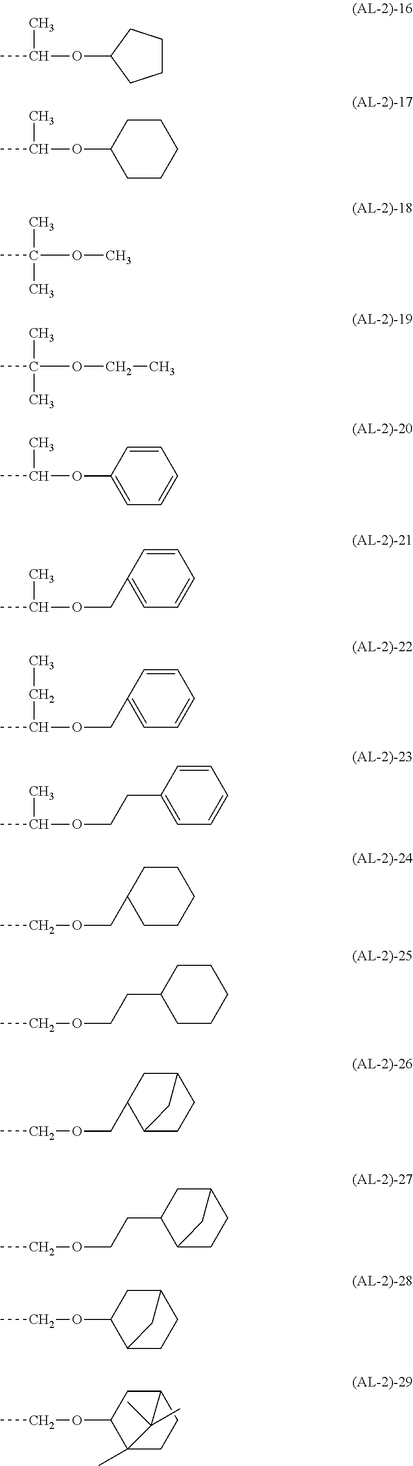

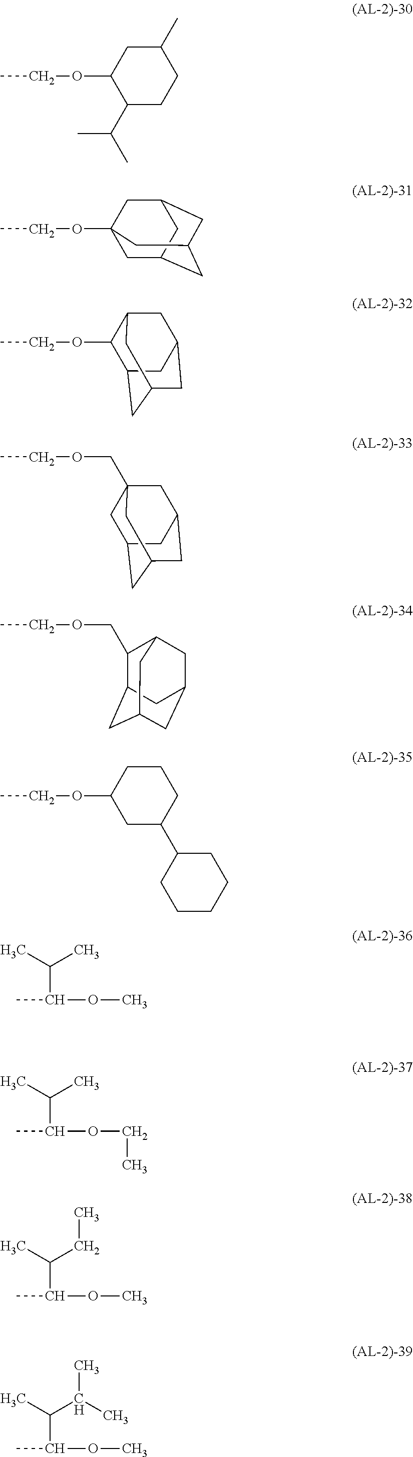

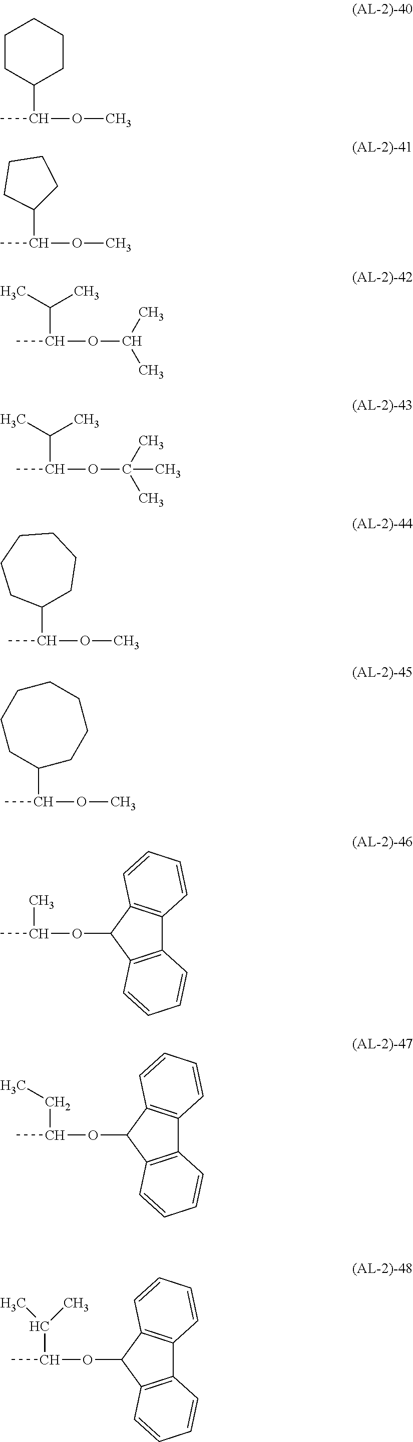

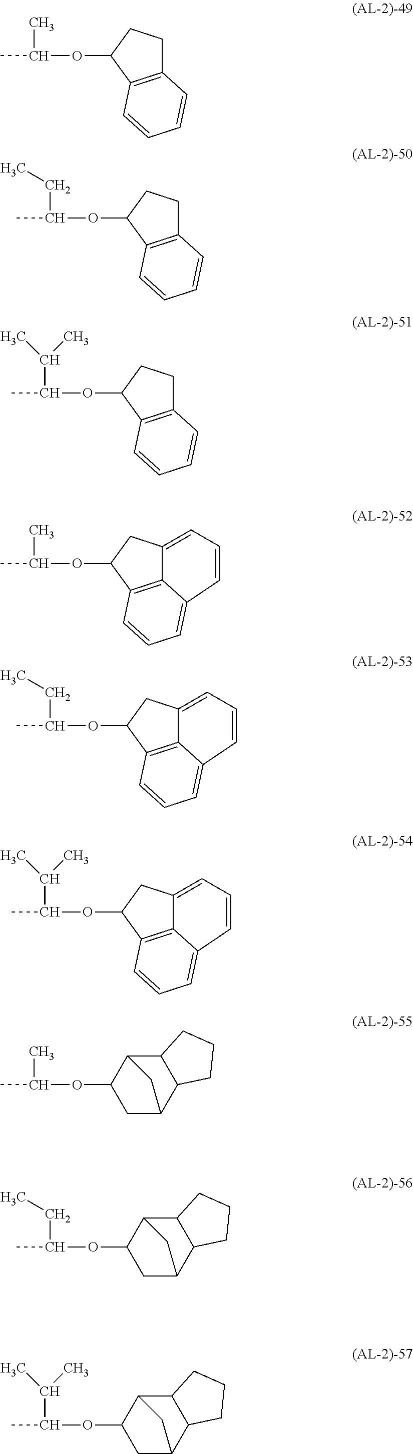

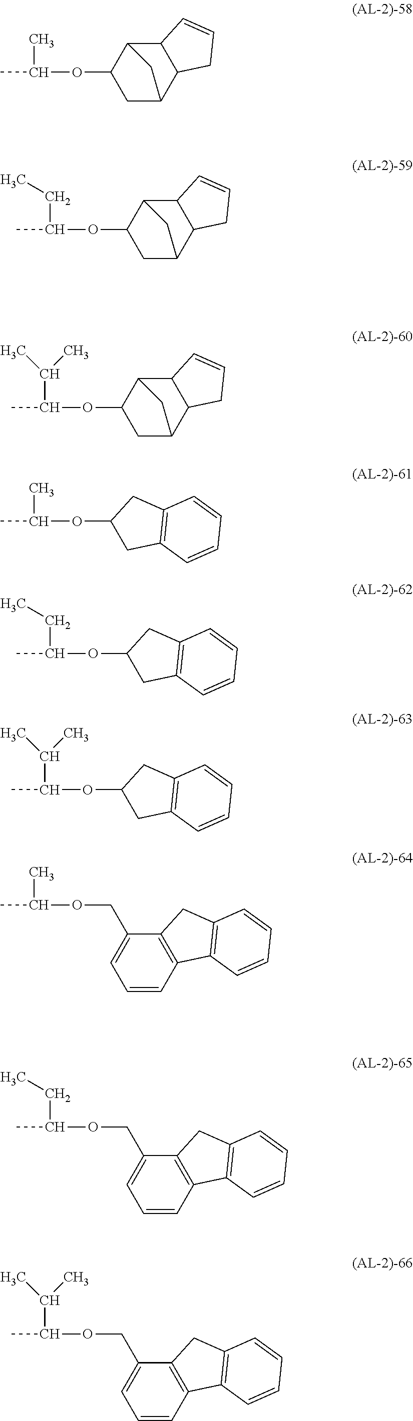

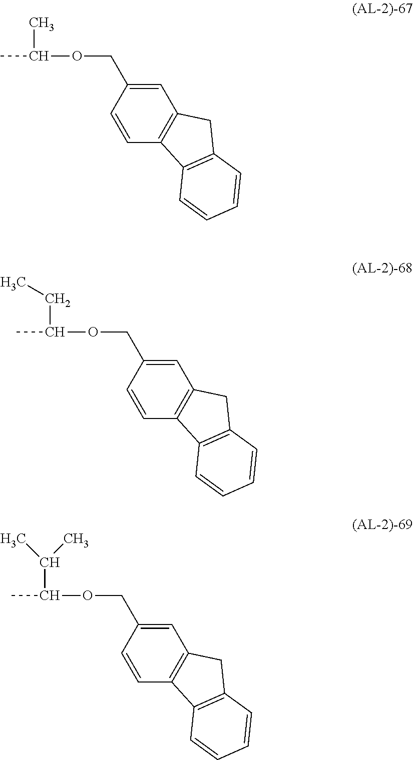

Examples of the cyclic ones among the acid-labile groups represented by the formula (AL-2) include a tetrahydrofuran-2-yl group, a 2-methyltetrahydrofuran-2-yl group, a tetrahydropyran-2-yl group, a 2-methyltetrahydropyran-2-yl group, etc.

In addition, examples of the acid-labile groups include groups represented by the following formula (AL-2a) or (AL-2b). The acid-labile group may crosslink the base polymer intermolecularly or intramolecularly.

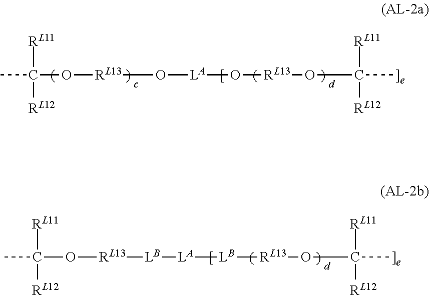

In the formulae, a broken line represents an attachment point.

In the formula (AL-2a) or (AL-2b), R^{L11} and R^{L12} each independently represent a hydrogen atom or a saturated hydrocarbyl group having 1 to 8 carbon atoms. The saturated hydrocarbyl group may be linear, branched, or cyclic. Alternatively, R^{L11} and R^{L12} may bond with each other to form a ring together with the carbon atom bonded therewith. In this case, R^{L11} and R^{L12} each independently represent an alkanediyl group having 1 to 8 carbon atoms. Each R^{L13} independently represents a saturated hydrocarbylene group having 1 to 10 carbon atoms. The saturated hydrocarbylene group may be linear, branched, or cyclic. “c” and “d” each independently represent an integer of 0 to 10, preferably an integer of 0 to 5, and “e” represents an integer of 1 to 7, preferably an integer of 1 to 3.

In the formula (AL-2a) or (AL-2b), L^{A} represents an aliphatic saturated hydrocarbon group having a valency of e+1 and having 1 to 50 carbon atoms, an alicyclic saturated hydrocarbon group having a valency of e+1 and having 3 to 50 carbon atoms, an aromatic hydrocarbon group having a valency of e+1 and having 6 to 50 carbon atoms, or a heterocyclic group having a valency of e+1 and having 3 to 50 carbon atoms. Furthermore, part of the —CH₂— of these groups may be substituted with a group containing a heteroatom, and part of the hydrogen atoms of these groups may be substituted with a hydroxy group, a carboxy group, an acyl group, or a fluorine atom. L^{A} is preferably a saturated hydrocarbon group, such as a saturated hydrocarbylene group, a trivalent saturated hydrocarbon group, and a tetravalent saturated hydrocarbon group, each of which has 1 to 20 carbon atoms, an arylene group having 6 to 30 carbon atoms, etc. The saturated hydrocarbon groups may be linear, branched, or cyclic. L^{B} represents —C(═O)—O—, —NH—C(═O)—O—, or —NH—C(═O)—NH—.

Examples of the crosslinking acetal group represented by the formula (AL-2a) or (AL-2b) include groups represented by the following formulae (AL-2)-70 to (AL-2)-77, etc.

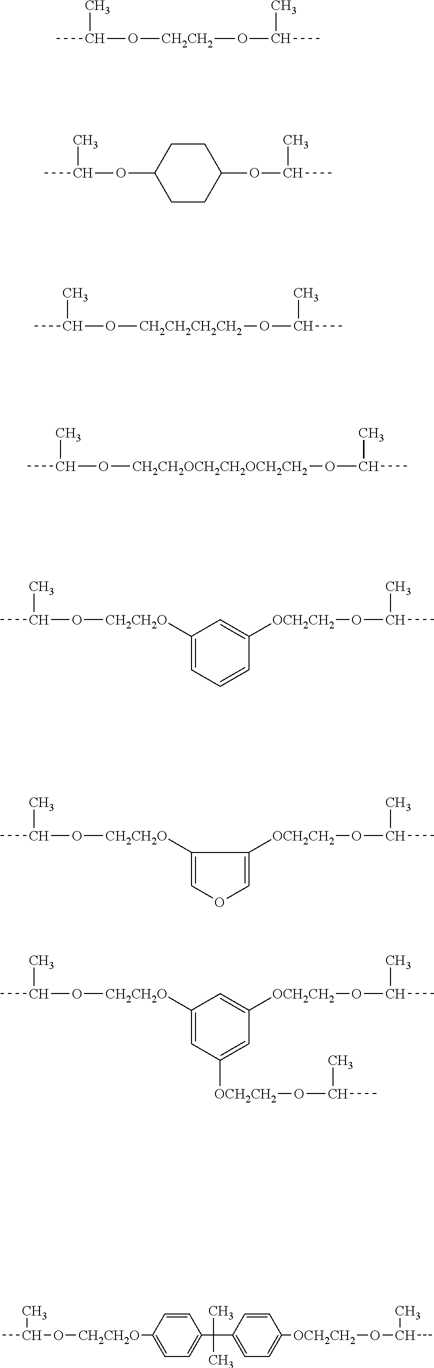

In the formulae, a broken line represents an attachment point.

In the formula (AL-3), R^{L5} represents a hydrogen atom or a hydrocarbyl group having 1 to 20 carbon atoms, and the hydrocarbyl group optionally contains a heteroatom, such as an oxygen atom, a sulfur atom, a nitrogen atom, and a fluorine atom. R^{L6} and R^{L7} each independently represent a hydrocarbyl group having 1 to 20 carbon atoms, and optionally contain a heteroatom, such as an oxygen atom, a sulfur atom, a nitrogen atom, and a fluorine atom. The hydrocarbyl group may be saturated or unsaturated, and may be linear, branched, or cyclic. Specific examples thereof include alkyl groups having 1 to 20 carbon atoms, cyclic saturated hydrocarbyl groups having 3 to 20 carbon atoms, alkenyl groups having 2 to 20 carbon atoms, cyclic unsaturated hydrocarbyl groups having 3 to 20 carbon atoms, aryl groups having 6 to 10 carbon atoms, etc. Alternatively, R^{L5} and R^{L6}, R^{L5} and R^{L7}, or R^{L6} and R^{L7} may bond with each other to form an alicyclic group having 3 to 20 carbon atoms, together with the carbon atom bonded therewith.

Examples of the group represented by the formula (AL-3) include a tert-butyl group, a 1,1-diethylpropyl group, a 1-ethylnorbornyl group, a 1-methylcyclopentyl group, a 1-ethylcyclopentyl group, a 1-isopropylcyclopentyl group, a 1-methylcyclohexyl group, a 2-(2-methyl)adamantyl group, a 2-(2-ethyl)adamantyl group, a tert-pentyl group, etc.

Examples of the group represented by the formula (AL-3) also include groups represented by the following formulae (AL-3)-1 to (AL-3)-22.

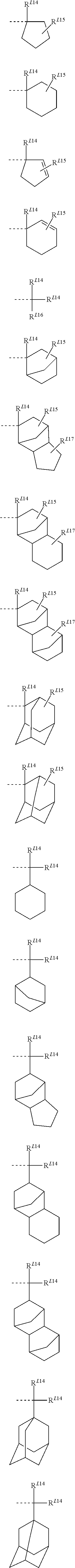

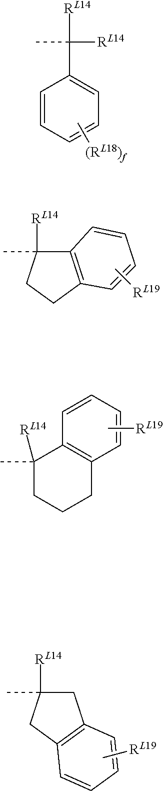

In the formulae, a broken line represents an attachment point.

In the formulae (AL-3)-1 to (AL-3)-22, each R^{L14} independently represents a hydrogen atom, an aliphatic hydrocarbyl group having 1 to 8 carbon atoms, or an aryl group having 6 to 20 carbon atoms. R^{L5} and R^{L17} each independently represent a hydrogen atom or a saturated hydrocarbyl group having 1 to 20 carbon atoms. R^{L16} represents an aryl group having 6 to 20 carbon atoms. The saturated hydrocarbyl groups may be linear, branched, or cyclic. Furthermore, as the aryl group, a phenyl group etc. are preferable. R^{L18} represents a fluorine atom, an iodine atom, a nitro group, or a trifluoromethyl group. Each R^{L19} independently represents a hydrogen atom, a fluorine atom, an iodine atom, a nitro group, a saturated hydrocarbyl group having 1 to 8 carbon atoms, or a hydrocarbyloxy group having 1 to 8 carbon atoms. “f” represents an integer of 1 to 5.

Examples of the acid-labile group further include groups represented by the following formula (AL-3)-23 or (AL-3)-24. The acid-labile group may crosslink the polymer intramolecularly or intermolecularly.

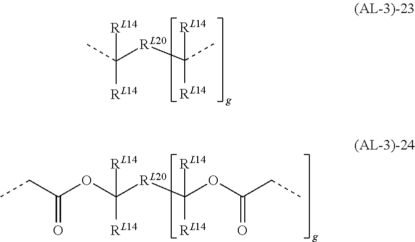

In the formulae, a broken line represents an attachment point.

In the formulae (AL-3)-23 and (AL-3)-24, R^{L14} is as defined above. R^{L20} represents a saturated or unsaturated hydrocarbylene group having a valency of g+1 and having 1 to 20 carbon atoms or represents an arylene group having a valency of g+1 and having 6 to 20 carbon atoms, and optionally contains a heteroatom such as an oxygen atom, a sulfur atom, and a nitrogen atom. The saturated or unsaturated hydrocarbylene group may be linear, branched, or cyclic. “g” represents an integer of 1 to 3.

Besides these acid-labile groups, it is also possible to use aromatic-group-containing acid-labile groups disclosed in JP5565293B2, JP5434983B2, JP5407941B2, JP5655756B2, and JP5655755B2.

The above-described polymer may include a repeating unit-c containing a phenolic hydroxy group as an adhesive group. Specific examples of a monomer to give the repeating unit-c include ones shown below, but are not limited thereto. Note that, in the following formulae, R^{A} is as defined above.

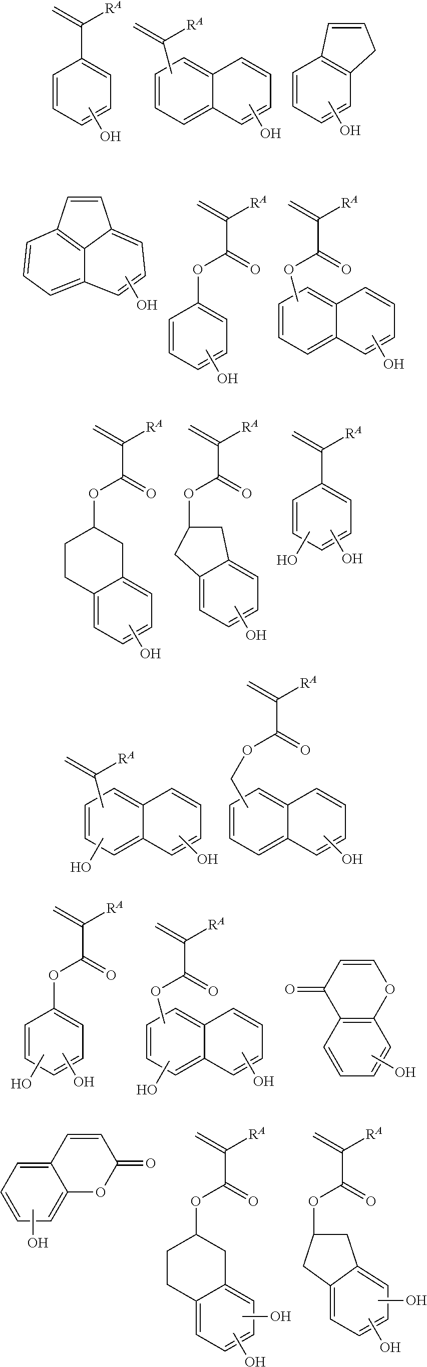

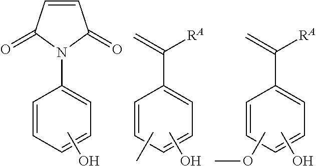

The polymer may include a repeating unit-d containing, as a different adhesive group, a hydroxy group other than a phenolic hydroxy group, a lactone ring, a sultone ring, an ether bond, an ester bond, a sulfonic acid ester bond, a carbonyl group, a sulfonyl group, a cyano group, or a carboxy group. Specific examples of a monomer to give the repeating unit-d include ones shown below, but are not limited thereto. Note that, in the following formulae, R^{A} is as defined above.

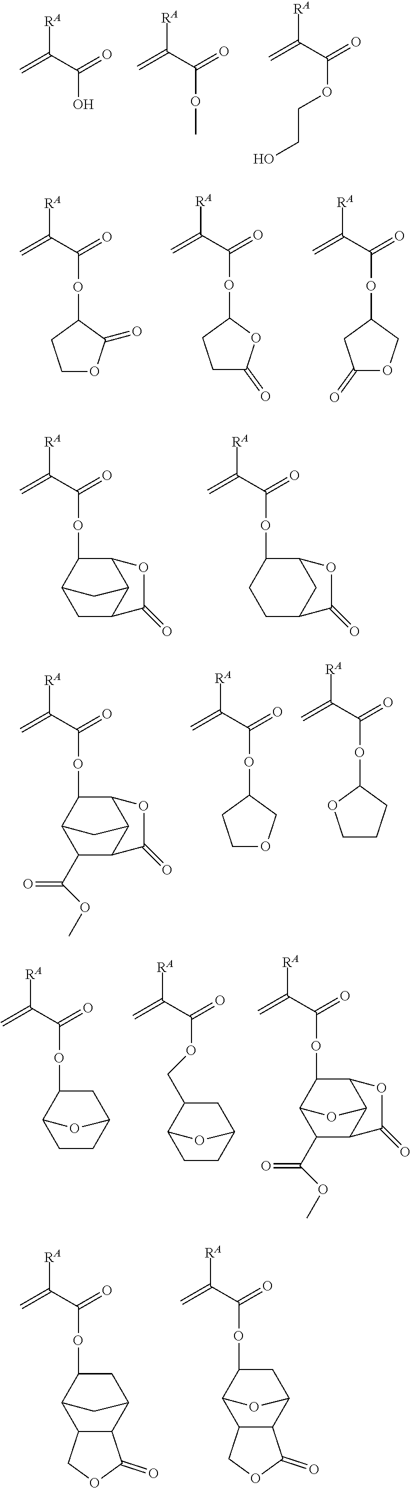

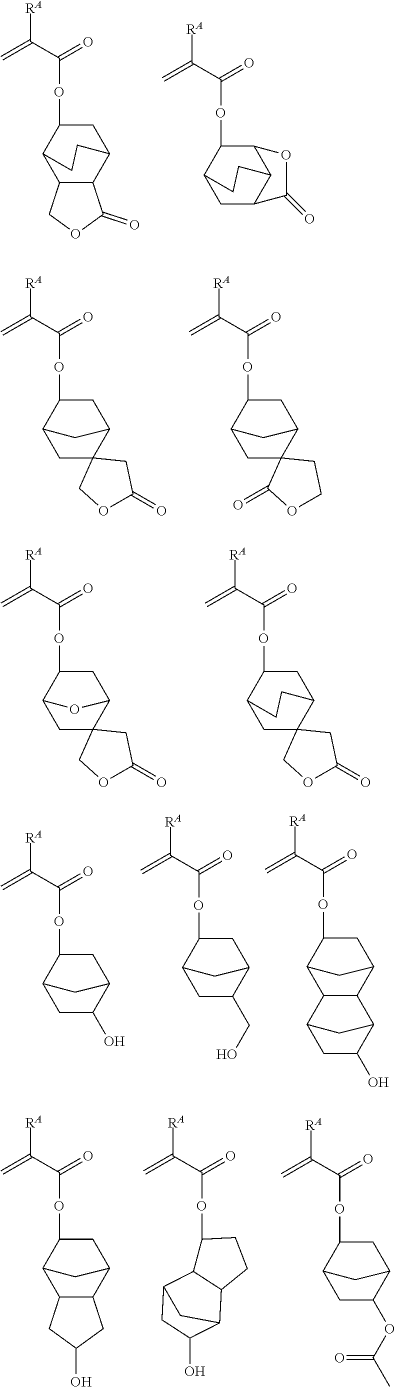

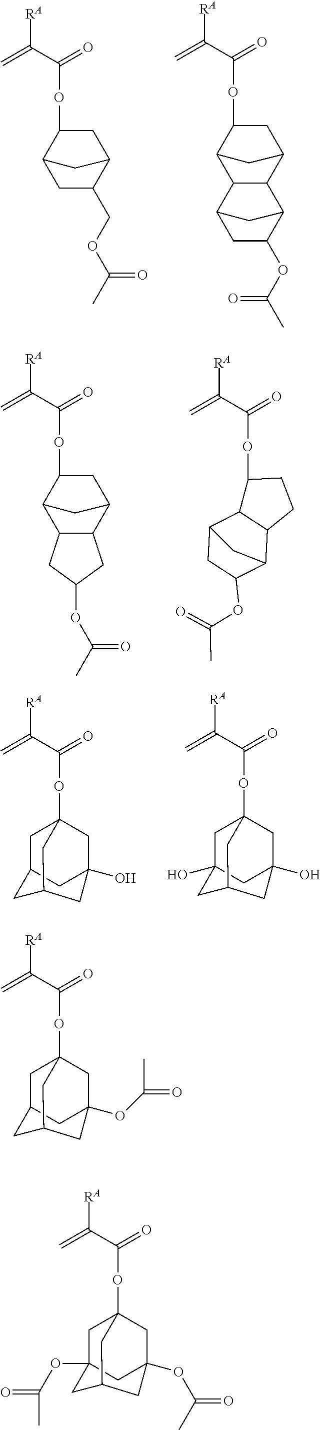

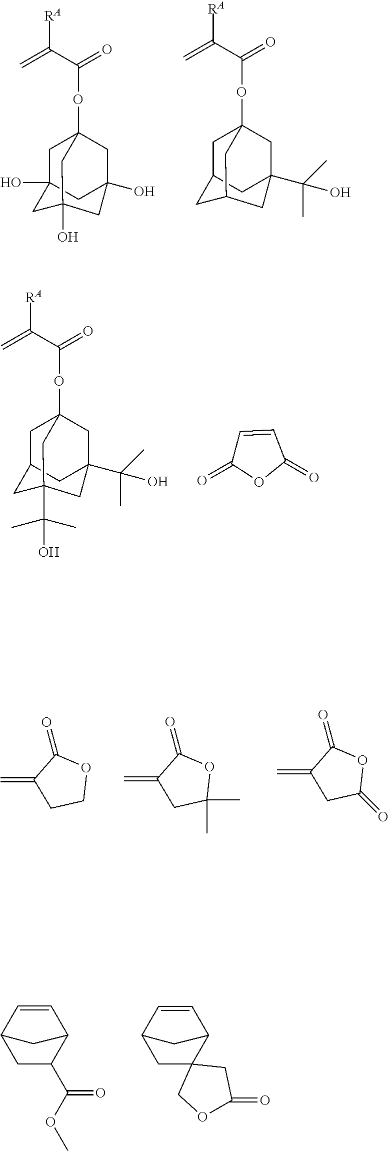

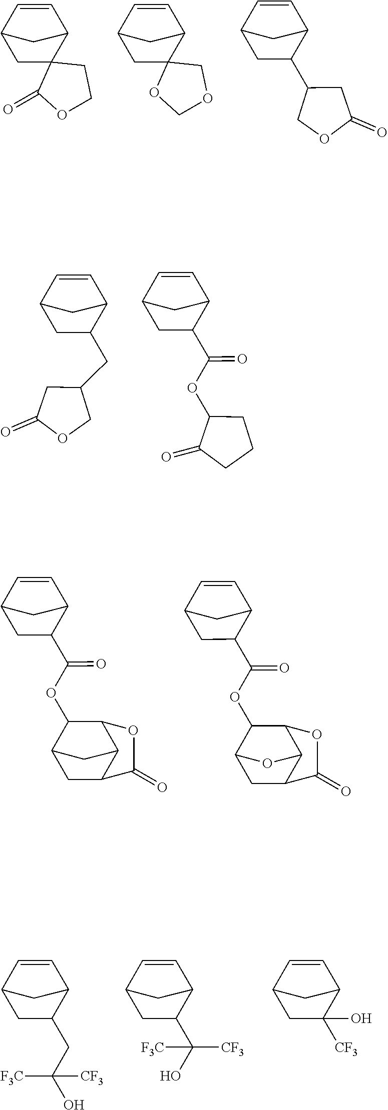

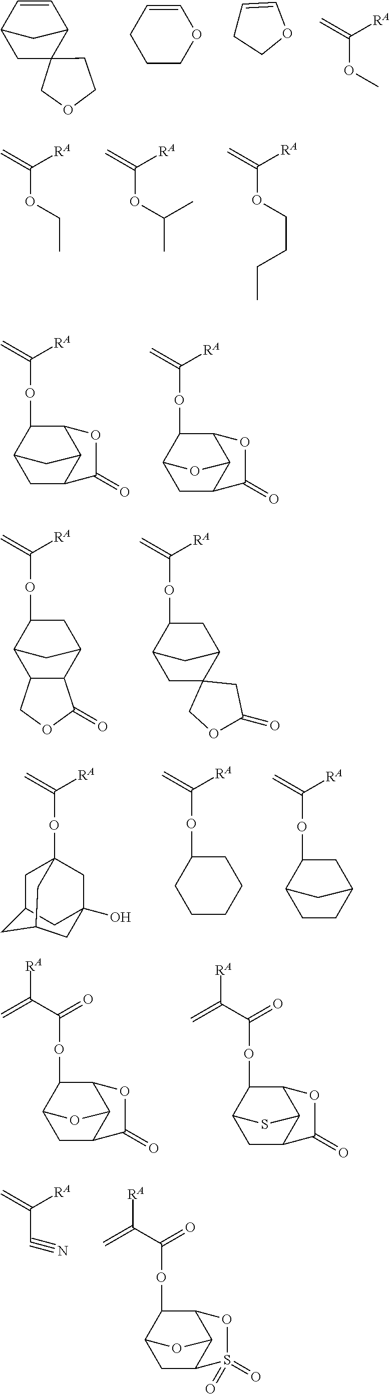

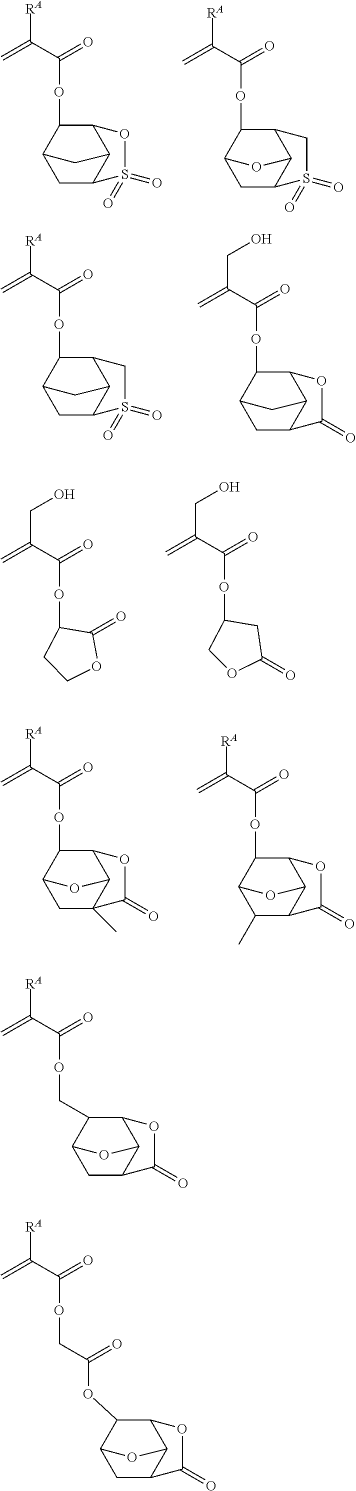

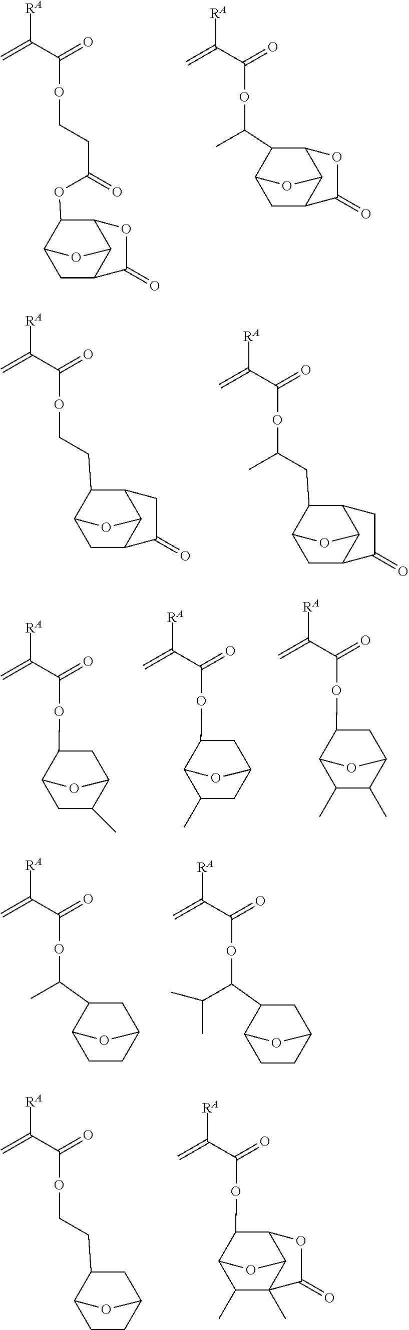

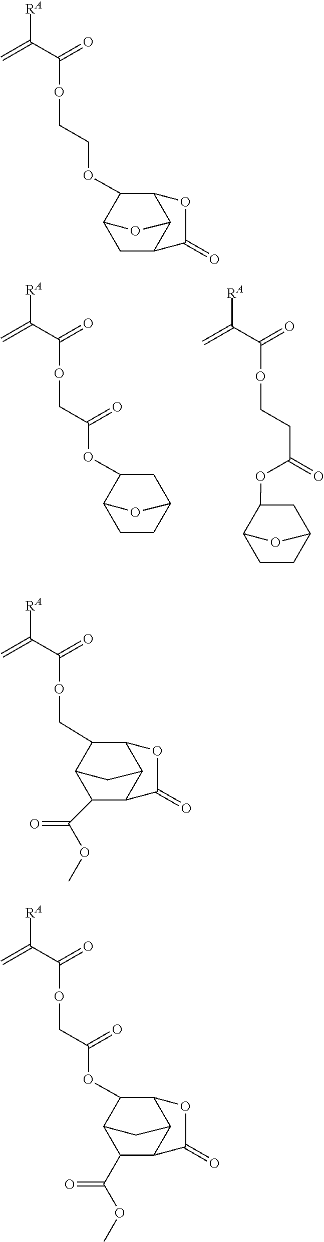

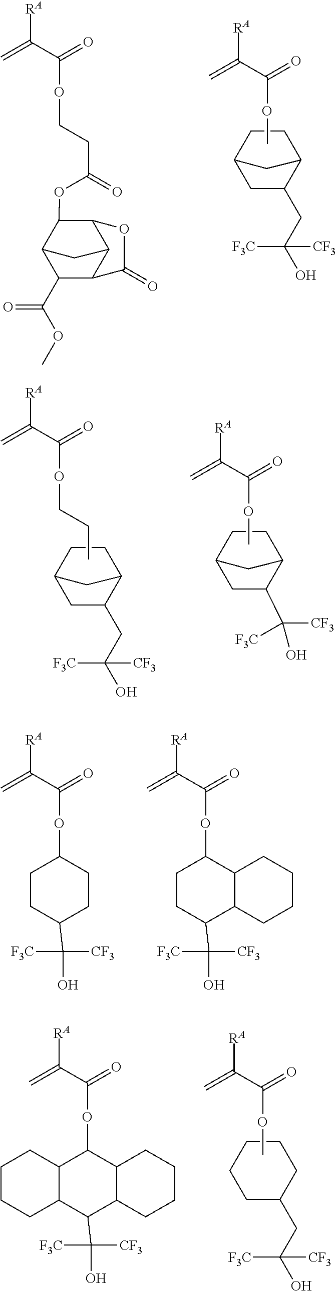

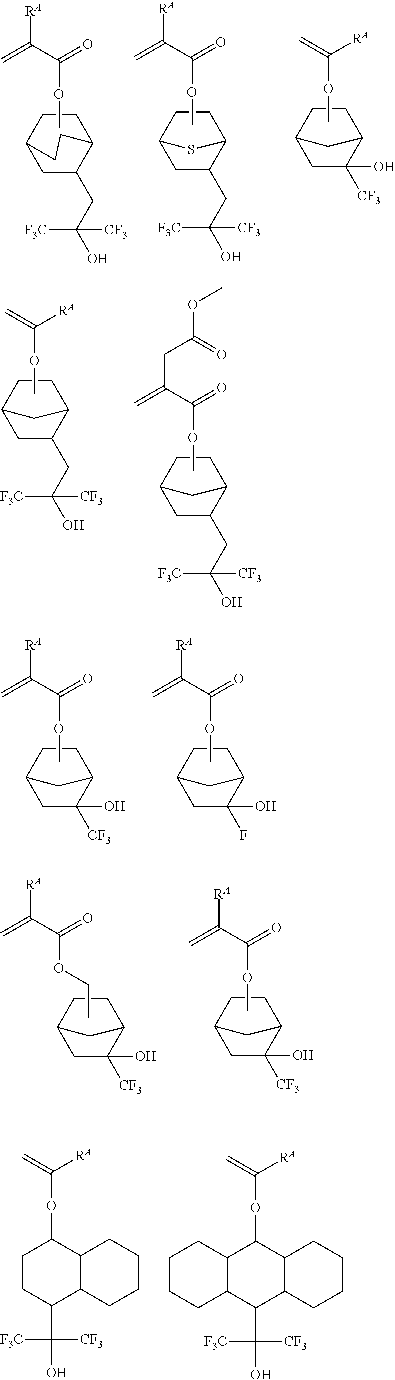

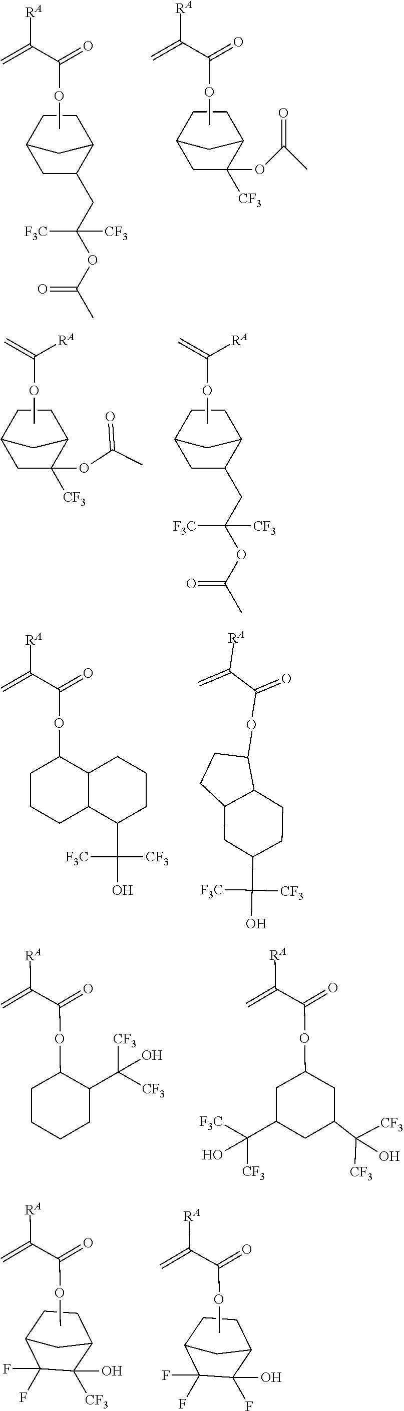

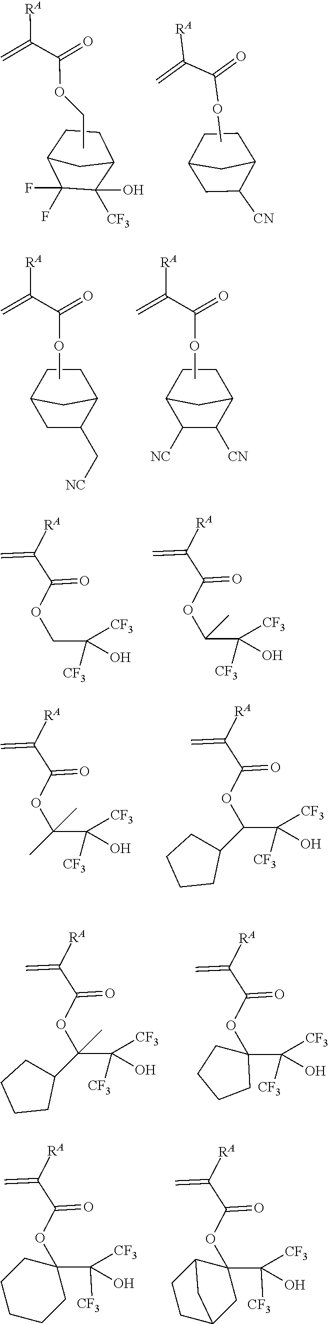

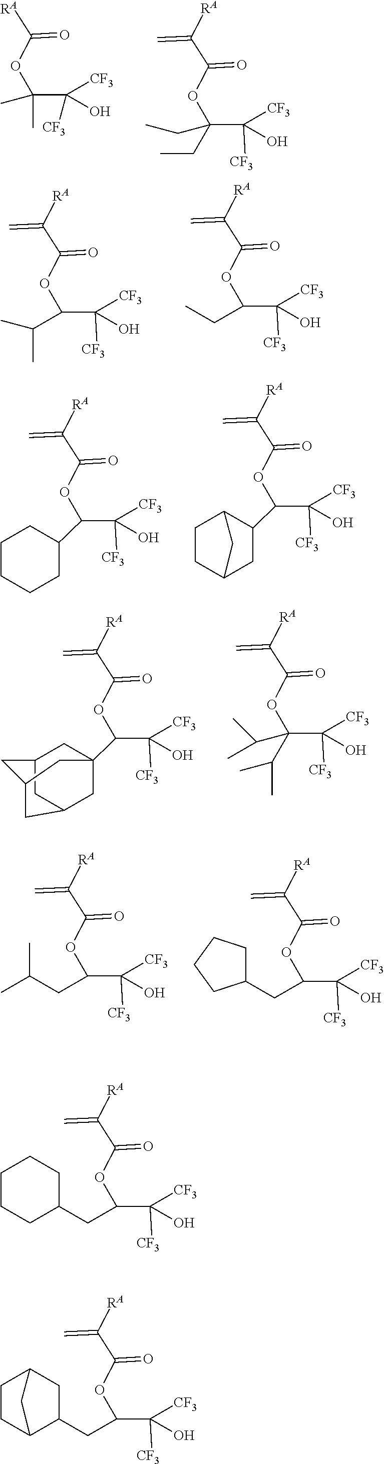

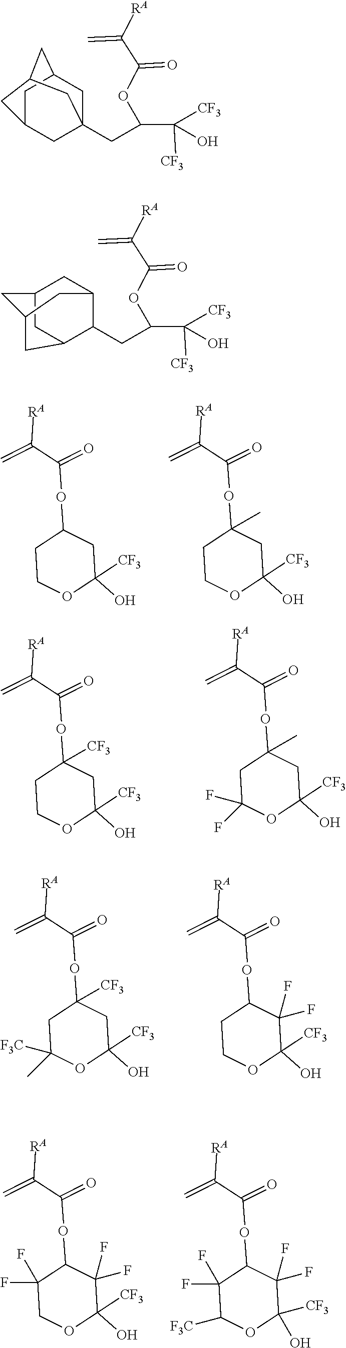

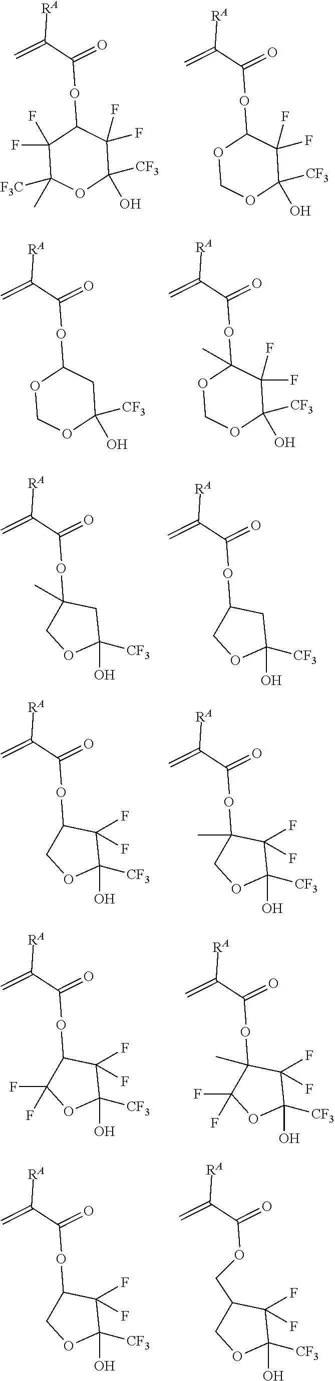

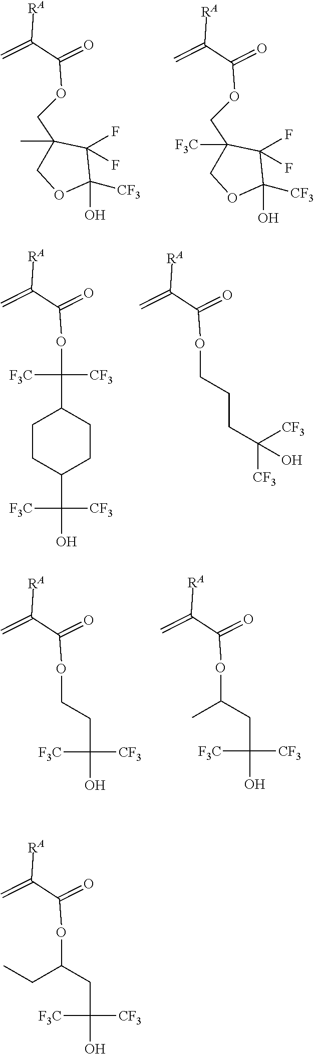

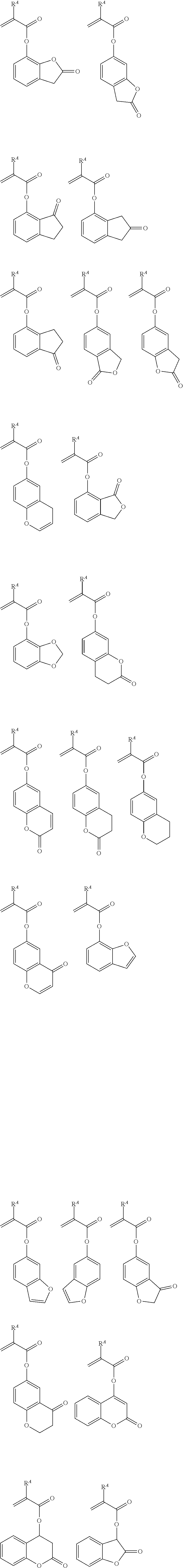

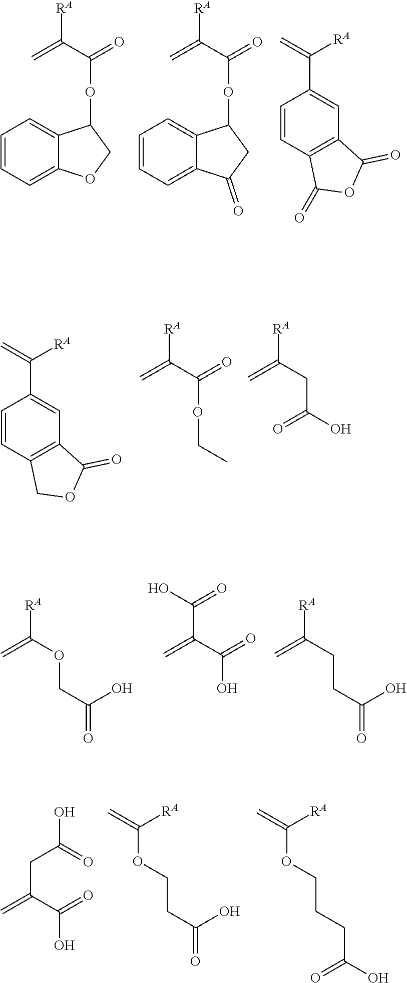

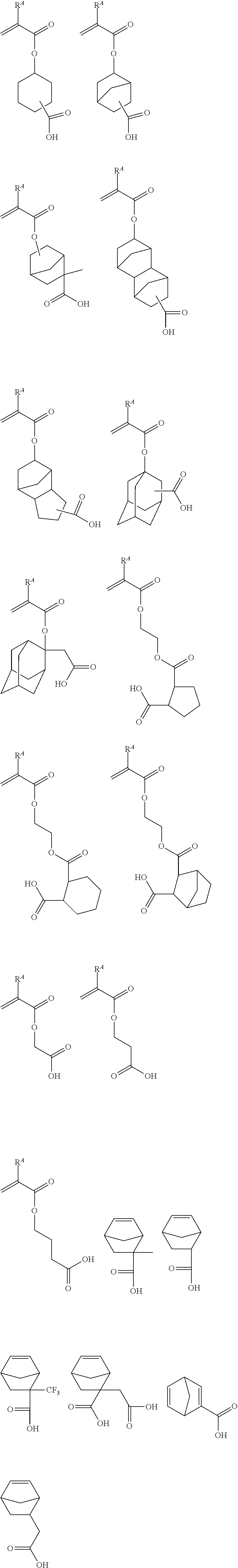

The polymer may include a repeating unit-e derived from indene, benzofuran, benzothiophene, acenaphthylene, chromone, coumarin, norbornadiene, or a derivative thereof. Specific examples of a monomer to give the repeating unit-e include ones shown below, but are not limited thereto.

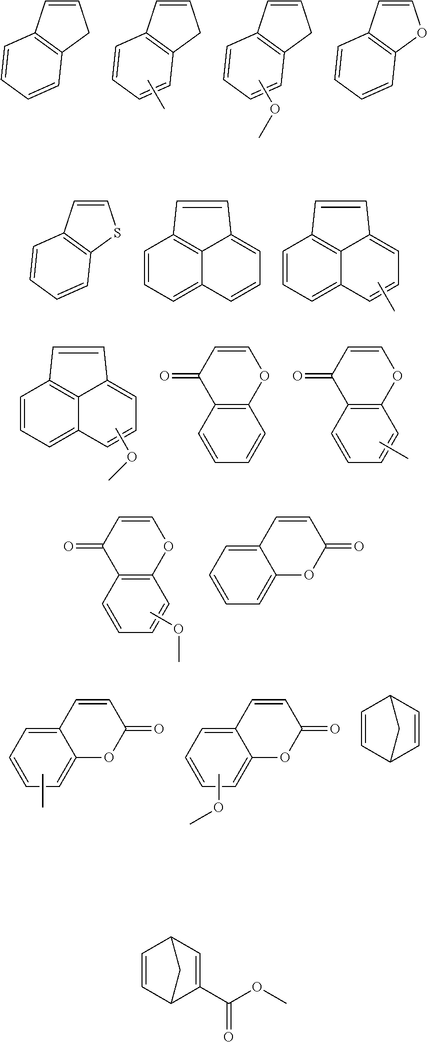

The polymer may include a repeating unit-f derived from styrene, vinylnaphthalene, vinylanthracene, vinylpyrene, methyleneindan, vinylpyridine, or vinylcarbazole.

In the inventive polymer, the content ratios of the repeating units-a, -b1, -b2, -c, -d, -e, and -f are preferably 0<a≤0.4, 0<b1≤0.8, 0≤b2≤0.8, 0<c≤0.6, 0≤d≤0.6, 0≤e≤0.3, and 0≤f≤0.3; more preferably 0<a≤0.3, 0<b1≤0.7, 0≤b2≤0.7, 0<c≤0.5, 0≤d≤0.5, 0≤e≤0.2, and 0≤f≤0.2, provided that a+b1+b2+c+d+e+f≤1.0.

When the inventive polymer is used as a base polymer for a positive resist material, the repeating unit-b1 or -b2, containing an acid-labile group, is essential to the inventive polymer in addition to the repeating unit-a. In this case, the content ratios of the repeating units-a, -b1, -b2, -c, -d, -e, and -f are preferably 0<a≤0.5, 0≤b1<1.0, 0≤b2<1.0, 0<b1+b2<1.0, 0≤c≤0.9, 0≤d≤0.9, 0≤e≤0.8, and 0≤f≤0.8; more preferably 0.02≤a≤0.4, 0≤b1≤0.9, 0≤b2≤0.9, 0.1≤b1+b2≤0.9, 0≤c≤0.8, 0≤d≤0.8, 0≤e≤0.7, and 0≤f≤0.7; and further preferably 0.05≤a≤0.35, 0≤b1≤0.8, 0≤b2≤0.8, 0.1≤b1+b2≤0.8, 0≤c≤0.75, 0≤d≤0.75, 0≤e≤0.6, and 0≤f≤0.6. In addition, a+b1+b2+c+d+e+f=1.0.

On the other hand, when the inventive polymer is used as a base polymer for a negative resist material, an acid-labile group is not necessarily needed. As such a base polymer, examples include ones including the repeating unit-c in addition to the repeating unit-a and further including the repeating unit-d, -e, and/or -f as necessary. The content ratios of these repeating units are preferably 0<a≤0.5, 0<c≤1.0, 0≤d≤0.9, 0≤e≤0.8, and 0≤f≤0.8; more preferably 0.02≤a≤0.4, 0.2≤c≤1.0, 0≤d≤0.8, 0≤e≤0.7, and 0≤f≤0.7; and further preferably 0.05≤a≤0.35, 0.3≤c≤1.0, 0≤d≤0.75, 0≤e≤0.6, and 0≤f≤0.6. In addition, a+c+d+e+f=1.0.

To synthesize the base polymer, for example, the monomers to give the repeating units described above can be subjected to heat polymerization in an organic solvent to which a radical polymerization initiator has been added.

Specific examples of the organic solvent used in the polymerization include toluene, benzene, tetrahydrofuran (THF), diethyl ether, and dioxane. Specific examples of the polymerization initiator include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. The temperature during the polymerization is preferably 50 to 80° C. The reaction time is preferably 2 to 100 hours, more preferably 5 to 20 hours.

In a case where a monomer containing a hydroxy group is copolymerized, it is possible to substitute the hydroxy group with an acetal group susceptible to deprotection with acid, such as an ethoxyethoxy group, prior to the polymerization and perform the deprotection with weak acid and water after the polymerization. Alternatively, it is possible to substitute the hydroxy group with an acetyl group, a formyl group, a pivaloyl group, or the like prior to the polymerization and perform alkaline hydrolysis after the polymerization.

In a case where hydroxystyrene or hydroxyvinylnaphthalene is copolymerized, acetoxystyrene or acetoxyvinylnaphthalene may be used in place of hydroxystyrene or hydroxyvinylnaphthalene; after the polymerization, the acetoxy group may be deprotected by the alkaline hydrolysis as described above to convert the acetoxystyrene or acetoxyvinylnaphthalene to hydroxystyrene or hydroxyvinylnaphthalene.

As a base in the alkaline hydrolysis, it is possible to use ammonia water, triethylamine, etc. In addition, the reaction temperature is preferably −20 to 100° C., more preferably 0 to 60° C. The reaction time is preferably 0.2 to 100 hours, more preferably 0.5 to 20 hours.

The above-described polymer preferably has a weight-average molecular weight (Mw) of 1000 to 500000, more preferably 2000 to 30000, and further preferably 5000 to 20000 in terms of polystyrene as measured by gel permeation chromatography (GPC) using THF or DMF as an eluent. When the Mw is in the above range, the heat resistance of the resist film and the solubility of the resist film in an alkaline developer are favorable. When GPC is measured, GPC is normally performed at a room temperature of around 23° C., but may be performed at a temperature higher or lower than this.

Furthermore, if the polymer has a wide molecular weight distribution (Mw/Mn), there are risks that foreign matters may be found on the pattern after the exposure and that pattern profile may be degraded due to low-molecular-weight and high-molecular-weight polymers being present. The finer the pattern rule, the stronger the influences of Mw and Mw/Mn. Hence, in order to obtain a resist material suitably used for finer pattern dimensions, the polymer preferably has a narrow dispersity Mw/Mn of 1.0 to 2.0, particularly preferably 1.0 to 1.5.

The polymer may include two or more polymers having different composition ratios, Mw, and Mw/Mn.

### [Chemically-Amplified Resist Composition]

The present invention also provides a chemically-amplified resist composition containing the above-described polymer as a base polymer. The proportion of the base polymer contained in the composition is not particularly limited, but can be, for example, 0.1 to 10 parts by mass, preferably 1 to 5 parts by mass, based on 100 parts by mass of the composition. In the following, components other than the base polymer contained in the inventive chemically-amplified resist composition will be described.

### [Organic Solvent]

The inventive chemically-amplified resist composition may contain an organic solvent. This organic solvent is not particularly limited, as long as it is capable of dissolving each of the above-described components and each of the components described below. Specific examples of the organic solvent include ones disclosed in paragraphs [0144] and [0145] of JP 2008-111103 A: ketones, such as cyclohexanone, cyclopentanone, methyl-2-n-pentyl ketone, and 2-heptanone; alcohols, such as 3-methoxybutanol, 3-methyl-3-methoxybutanol, 1-methoxy-2-propanol, 1-ethoxy-2-propanol, and diacetone alcohol; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones, such as γ-butyrolactone.

In the inventive chemically-amplified resist composition, the contained amount of the organic solvent is preferably 100 to 10000 parts by mass, more preferably 200 to 8000 parts by mass based on 100 parts by mass of the base polymer. One kind of the organic solvent may be used, or two or more kinds thereof may be used in mixture.

### [Quencher]

The inventive chemically-amplified resist composition may contain a quencher as necessary. In the present invention, a quencher means a compound that is capable of trapping an acid that is generated from a photo-acid generator to prevent the acid from diffusing to unexposed portions.

Specific examples of the quencher include amine compounds, sulfonate salts, or carboxylate salts. As the amine compounds, preferable are primary, secondary, or tertiary amine compounds disclosed in paragraphs [0146] to [0164] of JP2008-111103A; especially amine compounds having any of a hydroxy group, an ether bond, an ester bond, a lactone ring, a cyano group, a sulfonic acid ester bond, etc. In addition, a compound in which a primary or secondary amine is protected as a carbamate group, like the compound disclosed in JP3790649B2, is also preferable. Such a protected amine compound is effective when there is, in the resist composition, a component that is unstable to a base.

Specific examples of the sulfonate salts include compounds represented by the following formula (E1). Meanwhile, specific examples of the carboxylate salts include compounds represented by the following formula (E2).

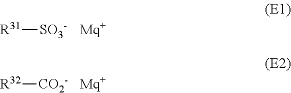

In the formula (E1), R³¹ represents a hydrogen atom or a hydrocarbyl group having 1 to 40 carbon atoms and optionally containing a heteroatom, but excludes groups in which a hydrogen atom bonded to the carbon atom at the a position of the sulfo group is substituted with a fluorine atom or a fluoroalkyl group.

The hydrocarbyl group having 1 to 40 carbon atoms represented by R³¹ may be saturated or unsaturated, and may be linear, branched, or cyclic. Specific examples thereof include: alkyl groups having 1 to 40 carbon atoms, such as a methyl group, an ethyl group, a propyl group, an isopropyl group, an n-butyl group, a sec-butyl group, a tert-butyl group, a tert-pentyl group, an n-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, an adamantyl group, and an adamantylmethyl group; alkenyl groups having 2 to 40 carbon atoms, such as a vinyl group, a 1-propenyl group, a 2-propenyl group, a butenyl group, and a hexenyl group; unsaturated aliphatic cyclic hydrocarbyl groups having 3 to 40 carbon atoms, such as a cyclohexenyl group; aryl groups having 6 to 40 carbon atoms, such as a phenyl group, a naphthyl group, alkylphenyl groups (a 2-methylphenyl group, a 3-methylphenyl group, a 4-methylphenyl group, a 4-ethylphenyl group, a 4-tert-butylphenyl group, a 4-n-butylphenyl group, etc.), dialkylphenyl groups (a 2,4-dimethylphenyl group a 2,4,6-triisopropylphenyl group, etc.), alkylnaphthyl groups (a methylnaphthyl group, an ethylnaphthyl group, etc.), and dialkylnaphthyl groups (a dimethylnaphthyl group, a diethylnaphthyl group, etc.); aralkyl groups having 7 to 40 carbon atoms, such as a benzyl group, a 1-phenylethyl group, and a 2-phenylethyl group; etc.

Moreover, part or all of the hydrogen atoms of the hydrocarbyl groups may be substituted with a group containing a heteroatom, such as an oxygen atom, a sulfur atom, a nitrogen atom, or a halogen atom, and part of the —CH₂— of the hydrocarbyl groups may be substituted with a group containing a heteroatom, such as an oxygen atom, a sulfur atom, or a nitrogen atom. The resulting hydrocarbyl groups may contain a hydroxy group, a fluorine atom, a chlorine atom, a bromine atom, an iodine atom, a cyano group, a carbonyl group, an ether bond, a thioether bond, an ester bond, a sulfonic acid ester bond, a carbonate bond, a lactone ring, a sultone ring, carboxylic anhydride (—C(═O)—O—C(═O)—), a haloalkyl group, etc. Examples of the hydrocarbyl group containing a heteroatom include: heteroaryl groups, such as a thienyl group; alkoxyphenyl groups, such as a 4-hydroxyphenyl group, a 4-methoxyphenyl group, a 3-methoxyphenyl group, a 2-methoxyphenyl group, a 4-ethoxyphenyl group, a 4-tert-butoxyphenyl group, and a 3-tert-butoxyphenyl group; alkoxynaphthyl groups, such as a methoxynaphthyl group, an ethoxynaphthyl group, an n-propoxynaphthyl group, and an n-butoxynaphthyl group; dialkoxynaphthyl groups, such as a dimethoxynaphthyl group and a diethoxynaphthyl group; aryloxoalkyl groups, such as 2-aryl-2-oxoethyl groups including a 2-phenyl-2-oxoethyl group, a 2-(1-naphthyl)-2-oxoethyl group, and a 2-(2-naphthyl)-2-oxoethyl group; etc.

R³² represents a hydrocarbyl group having 1 to 40 carbon atoms and optionally containing a heteroatom. Examples of the hydrocarbyl group represented by R³² include those given as examples of the hydrocarbyl group represented by R³¹. Furthermore, other specific examples thereof include fluorine-containing alkyl groups, such as a trifluoromethyl group, a trifluoroethyl group, a 2,2,2-trifluoro-1-methyl-1-hydroxyethyl group, and a 2,2,2-trifluoro-1-(trifluoromethyl)-1-hydroxyethyl group; fluorine-containing aryl groups, such as a pentafluorophenyl group and a 4-trifluoromethylphenyl group; etc.

Specific examples of the anion of the sulfonate salt represented by the formula (E1) include ones shown below, but are not limited thereto.

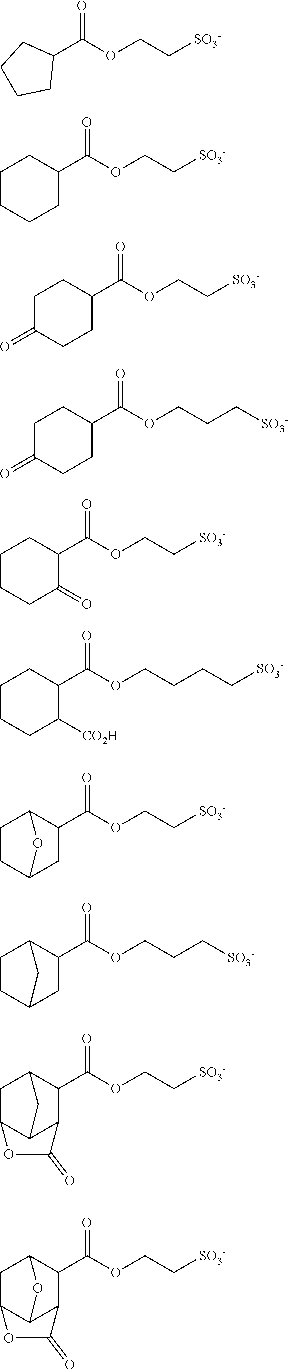

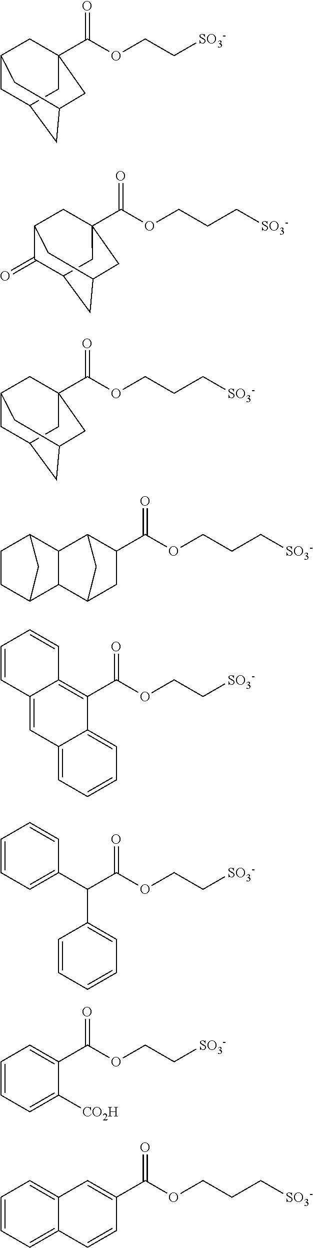

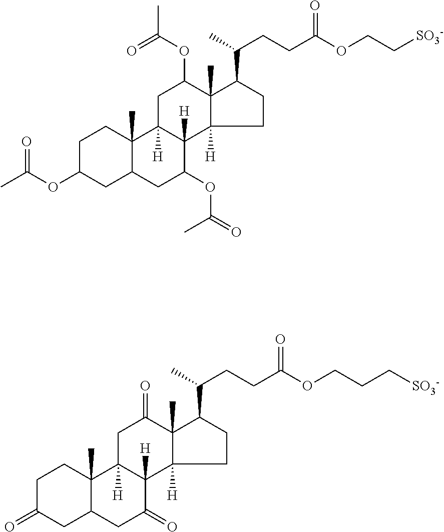

Specific examples of the anion of the carboxylate salt represented by the formula (E2) include ones shown below, but are not limited thereto.

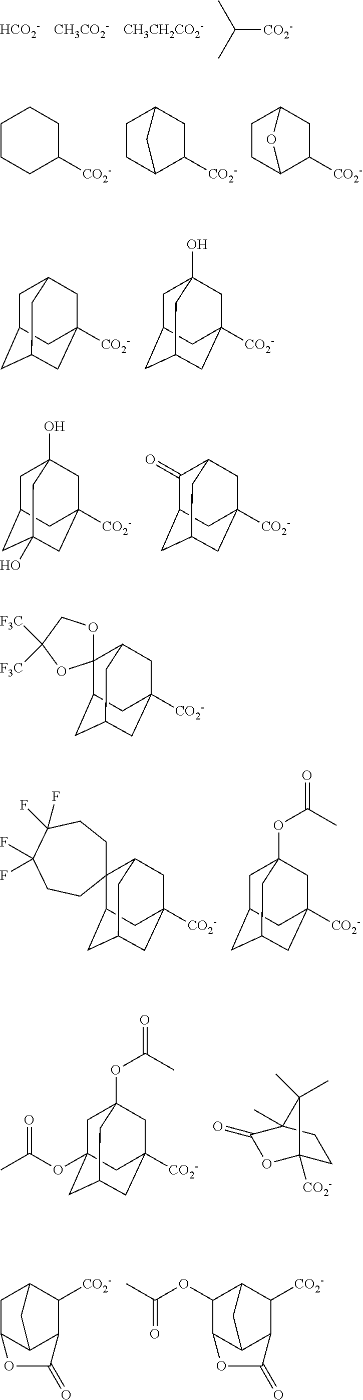

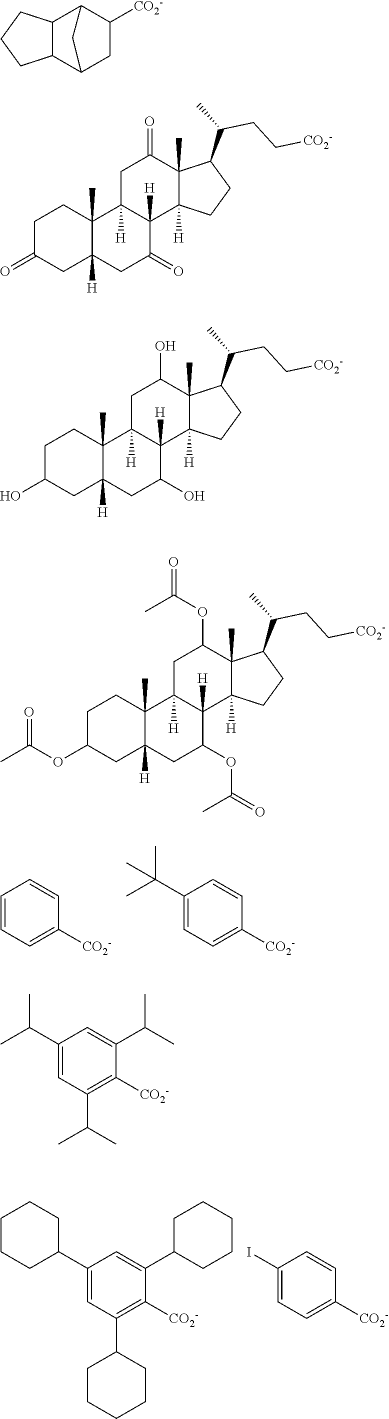

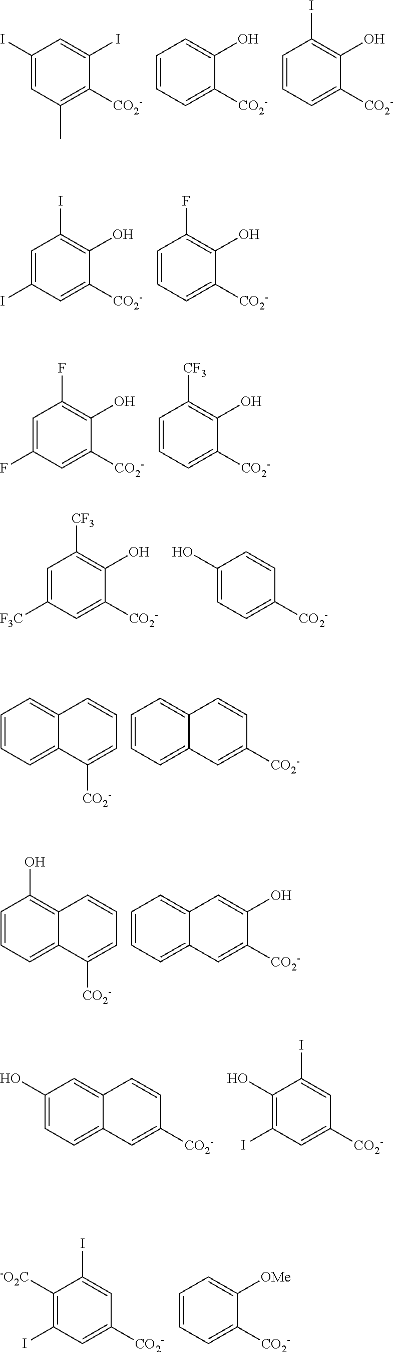

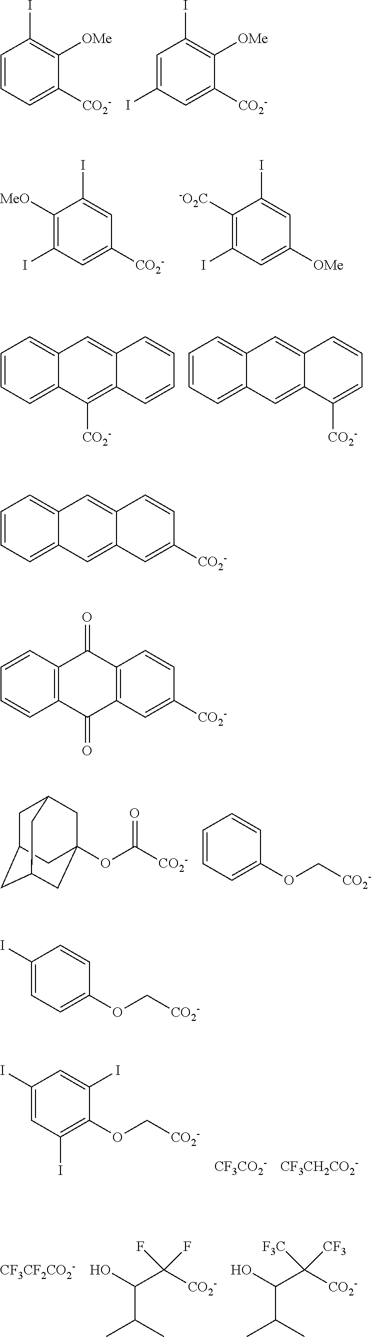

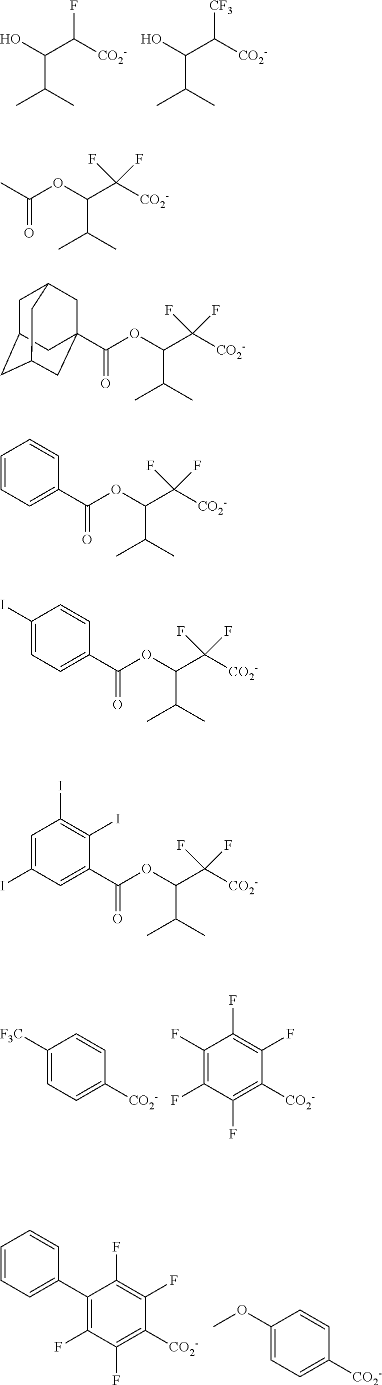

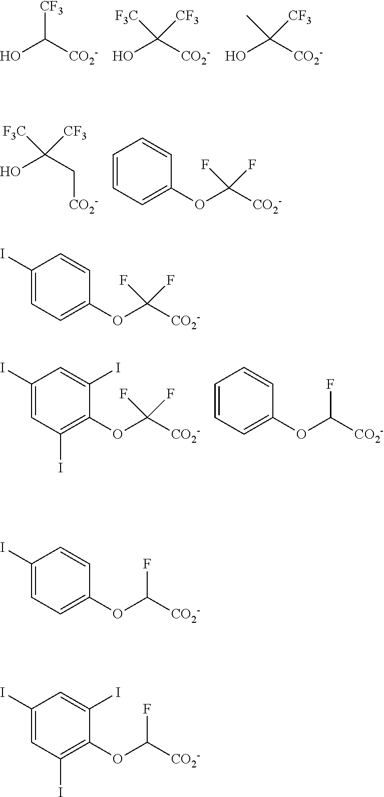

In the formulae (E1) and (E2), Mq⁺ represents an onium cation. Examples of the onium cation include a sulfonium cation, an iodonium cation, an ammonium cation, etc. As the sulfonium cation, ones represented by the formula (Z-1) are preferable, and specific examples thereof include ones given as specific examples of the sulfonium cation represented by the formula (Z-1). As the iodonium cation, ones represented by the formula (Z-2) are preferable, and specific examples thereof include ones given as specific examples of the iodonium cation represented by the formula (Z-2).

Other examples of the quencher include polymer-type quenchers disclosed in JP2008-239918A. The quenchers enhance the rectangularity of a resist pattern by being oriented on the resist surface. The polymer-type quencher also has effects of preventing rounding of a pattern top and film thickness loss of a pattern when a top coat for immersion exposure is applied.

Furthermore, as the quencher, it is also possible to use: an onium salt disclosed in JP6848776B2, having a sulfonium cation and a phenoxide anion moiety within a single molecule; furthermore, an onium salt disclosed in JP6583136B2 or JP2020-200311A, having a sulfonium cation and a carboxylate anion moiety within a single molecule; or an onium salt disclosed in JP6274755B2, having an iodonium cation and a carboxylate anion moiety within a single molecule.

When the inventive chemically-amplified resist composition contains the quencher, the contained amount is preferably 0.001 to 12 parts by mass, more preferably 0.01 to 8 parts by mass, based on 80 parts by mass of the base polymer. By the quencher being contained, adjustment of the sensitivity of the resist film is facilitated, and in addition, diffusion of acid in the resist film is suppressed and resolution is enhanced. Thus, it is possible to suppress change in sensitivity after exposure and reduce dependency on the substrate and the environment, making it possible to enhance exposure latitude, pattern profile, etc. Furthermore, by adding the quencher, it is also possible to enhance substrate adhesiveness. One kind of the quencher may be used, or two or more kinds thereof may be used in combination.

### [Other Components]

In addition to the above-described components, an acid generator, a surfactant, a dissolution inhibitor, a crosslinking agent, a water-repellency enhancer, an acetylene alcohol, etc. may be contained.

Examples of the acid generator include compounds that generate acids in response to actinic light or radiation (photo-acid generators). The photo-acid generator component is not particularly limited, as long as the compound generates an acid upon irradiation with a high-energy beam. An acid generator that generates a sulfonic acid, imide acid, or methide acid is preferable. Specific examples of suitable photo-acid generators include sulfonium salt, iodonium salt, sulfonyldiazomethane, N-sulfonyloxyimide, oxime-O-sulfonate type acid generators, etc. Specific examples of the acid generator include ones disclosed in paragraphs [0122] to [0142] of JP2008-111103A, JP2018-5224A, and JP2018-25789A. When the inventive chemically-amplified resist composition contains the acid generator, the contained amount is preferably 0 to 200 parts by mass, preferably 0.1 to 100 parts by mass, based on 100 parts by mass of the base polymer. Note that, since the inventive polymer includes a base polymer that functions as a photo-acid generator, it is not essential for the other acid generators to be additionally contained.

Specific examples of the surfactant include ones disclosed in paragraphs [0165] and [0166] of JP2008-111103A. Adding a surfactant can further enhance or control the coatability of the chemically-amplified resist composition. When the inventive chemically-amplified resist composition contains the surfactant, the contained amount is preferably 0.0001 to 10 parts by mass based on 100 parts by mass of the base polymer. One kind of the surfactant may be used, or two or more kinds thereof may be used in combination.

When the inventive chemically-amplified resist composition is a positive type, blending a dissolution inhibitor can further increase the difference in dissolution rate between exposed and unexposed portions, and further enhance the resolution. Specific examples of the dissolution inhibitor include a compound which contains two or more phenolic hydroxy groups in a molecule, and in which 0 to 100 mol % of all the hydrogen atoms of the phenolic hydroxy groups are substituted with acid-labile groups; or a compound which contains a carboxy group in a molecule, and in which 50 to 100 mol % of all the hydrogen atoms of such carboxy groups are substituted with acid-labile groups on average. In addition, the compounds each have a molecular weight of preferably 100 to 1000, more preferably 150 to 800. Specific examples include compounds obtained by substituting acid-labile groups for hydrogen atoms of hydroxy groups or carboxy groups of bisphenol A, trisphenol, phenolphthalein, cresol novolak, naphthalenecarboxylic acid, adamantanecarboxylic acid, or cholic acid; etc. Examples are disclosed in paragraphs [0155] to [0178] of JP 2008-122932 A.

When the inventive chemically-amplified resist composition is a positive type and contains the dissolution inhibitor, the contained amount is preferably 0 to 50 parts by mass, more preferably 5 to 40 parts by mass based on 100 parts by mass of the base polymer. One kind of the dissolution inhibitor can be used, or two or more kinds thereof can be used in combination.

Meanwhile, when the inventive chemically-amplified resist composition is a negative type, a negative pattern can be obtained by reducing the dissolution rate in exposed portions by adding a crosslinking agent. Specific examples of the crosslinking agent include: epoxy compounds, melamine compounds, guanamine compounds, and glycoluril compounds, each substituted with at least one group selected from a methylol group, an alkoxymethyl group, and an acyloxymethyl group; or compounds including a double bond, such as urea compounds, isocyanate compounds, azide compounds, and alkenyloxy groups. These may be contained as additives, but may also be introduced as a pendant group in a polymer side chain. A compound containing a hydroxy group can also be contained as a crosslinking agent.

Specific examples of the epoxy compounds include tris(2,3-epoxypropyl) isocyanurate, trimethylolmethane triglycidyl ether, trimethylolpropane triglycidyl ether, and triethylolethane triglycidyl ether.

Specific examples of the melamine compounds include hexamethylolmelamine, hexamethoxymethylmelamine, and compounds in which 1 to 6 methylol groups of hexamethylolmelamine have been methoxymethylated or mixtures thereof; hexamethoxymethylmelamine, hexaacyloxymethylmelamine, and compounds in which 1 to 6 methylol groups of hexamethylolmelamine have been acyloxymethylated or mixtures thereof.

Specific examples of the guanamine compounds include: tetramethylolguanamine, tetramethoxymethylguanamine, and compounds in which 1 to 4 methylol groups of tetramethylolguanamine have been methoxymethylated or mixtures thereof; and tetramethoxyethylguanamine, tetraacyloxyguanamine, and compounds in which 1 to 4 methylol groups of tetramethylolguanamine have been acyloxymethylated or mixtures thereof.

Specific examples of the glycoluril compounds include: tetramethylolglycoluril, tetramethoxyglycoluril, tetramethoxymethylglycoluril, and compounds in which 1 to 4 methylol groups of tetramethylolglycoluril have been methoxymethylated or mixtures thereof; and compounds in which 1 to 4 methylol groups of tetramethylolglycoluril have been acyloxymethylated or mixtures thereof. Specific examples of the urea compounds include: tetramethylurea, tetramethoxymethylurea, and compounds in which 1 to 4 methylol groups of tetramethylurea have been methoxymethylated or mixtures thereof; and tetramethoxyethylurea.

Specific examples of the isocyanate compounds include tolylenediisocyanate, diphenylmethanediisocyanate, hexamethylenediisocyanate, and cyclohexanediisocyanate.

Specific examples of the azide compounds include 1,1′-biphenyl-4,4′-bisazide, 4,4′-methylidenebisazide, and 4,4′-oxybisazide.

Specific examples of the compounds containing an alkenyloxy group include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylolpropane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylolpropane trivinyl ether.

When the inventive chemically-amplified resist composition is a negative type and contains the crosslinking agent, the contained amount is preferably 0.1 to 50 parts by mass, more preferably 1 to 40 parts by mass, based on 100 parts by mass of the base polymer. One kind of the crosslinking agent may be used, or two or more kinds thereof may be used in combination.

The water-repellency enhancer improves the water-repellency of the resist film surface, and can be employed in immersion lithography with no top coat. The water-repellency enhancer is preferably a polymer containing a fluorinated alkyl group, a polymer containing a 1,1,1,3,3,3-hexafluoro-2-propanol residue with a particular structure, etc., preferably ones exemplified in JP2007-297590A, JP2008-111103A, etc. The water-repellency enhancer needs to be dissolved in an alkaline developer or an organic solvent developer. The water-repellency enhancer having a particular 1,1,1,3,3,3-hexafluoro-2-propanol residue mentioned above has favorable solubility to developers. A polymer including a repeating unit with an amino group or amine salt as a water-repellency enhancer exhibits high effects of preventing acid evaporation during PEB to prevent opening failure of a hole pattern after development. When the inventive chemically-amplified resist composition contains the water-repellency enhancer, the contained amount is preferably 0 to 20 parts by mass, more preferably 0.5 to 10 parts by mass based on 100 parts by mass of the base polymer. One kind of the water-repellency enhancer may be used, or two or more kinds thereof may be used in combination.

Specific examples of the acetylene alcohol include ones disclosed in paragraphs [0179] to [0182] of JP2008-122932A. When the inventive chemically-amplified resist composition contains the acetylene alcohol, the contained amount is preferably 0 to 5 parts by mass based on 100 parts by mass of the base polymer. One kind of the acetylene alcohol may be used, or two or more kinds thereof may be used in combination.

### [Patterning Process]

When the inventive chemically-amplified resist composition is used for manufacturing various integrated circuits, known lithography techniques can be applied. Examples of the patterning process include a method including the steps of: forming a resist film on a substrate by using the above-described chemically-amplified resist composition; exposing the resist film by using a high-energy beam; and developing the exposed resist film by using a developer.

Firstly, the inventive chemically-amplified resist composition is applied onto a substrate (Si, SiO₂, SiN, SiON, TiN, WSi, BPSG, SOG, organic antireflective film, etc.) for manufacturing an integrated circuit or a substrate (Cr, CrO, CrON, MoSi₂, SiO₂, etc.) for manufacturing a mask circuit by an appropriate coating process, such as spin coating, roll coating, flow coating, dip coating, spray coating, and doctor coating, so that the coating film has a thickness of 0.01 to 2 μm. The coating film is prebaked on a hot plate preferably at 60 to 150° C. for 10 seconds to 30 minutes, more preferably at 80 to 120° C. for 30 seconds to 20 minutes. Thus, a resist film is formed.

Then, the resist film is exposed using a high-energy beam. Specific examples of the high-energy beam include ultraviolet ray, deep ultraviolet ray, EB (electron beam), EUV (extreme ultraviolet ray) having a wavelength of 3 to 15 nm, X-ray, soft X-ray, excimer laser beam, γ-ray, synchrotron radiation, etc. When ultraviolet ray, deep ultraviolet ray, EUV, X-ray, soft X-ray, excimer laser beam, γ-ray, synchrotron radiation, or the like is employed as the high-energy beam, the irradiation is performed directly or while using a mask for forming a target pattern at an exposure dose of preferably about 1 to 200 mJ/cm², more preferably about 10 to 100 mJ/cm². When EB is employed as the high-energy beam, the exposure dose is preferably about 0.1 to 300 μC/cm², more preferably about 0.5 to 200 μC/cm², and the writing is performed directly or while using a mask for forming a target pattern. Incidentally, the inventive chemically-amplified resist composition is particularly suitable for fine patterning with a KrF excimer laser beam, having a wavelength of 248 nm, an ArF excimer laser beam, having a wavelength of 193 nm, an EB, EUV, X-ray, soft X-ray, γ-ray, or synchrotron radiation among the high-energy beams, and is particularly suitable for fine patterning with an EB or EUV.

After the exposure, PEB may be performed on a hot plate or in an oven, preferably at 30 to 150° C. for 10 seconds to 30 minutes, more preferably 50 to 120° C. for 30 seconds to 20 minutes. Alternatively, PEB may not be performed.

After the exposure or PEB, the exposed resist film can be developed using a developer of 0.1 to 10 mass %, preferably 2 to 5 mass %, aqueous alkaline solution of a material, such as tetramethylammonium hydroxide (TMAH), tetraethylammonium hydroxide, tetrapropylammonium hydroxide, and tetrabutylammonium hydroxide, for 3 seconds to 3 minutes, preferably 5 seconds to 2 minutes, by a conventional technique, such as a dip method, a puddle method, or a spray method. Thereby, the target pattern is formed. In the case of a positive resist material, the portion irradiated with the light is dissolved by the developer, while the unexposed portion remains undissolved. Thus, the target positive pattern is formed on the substrate. A case with a negative resist material is the reverse of the case with the positive resist material, and the portion irradiated with the light becomes insoluble in a developer, while the unexposed portion dissolves.

It is also possible to obtain a negative pattern by organic solvent development using a positive resist material containing a base polymer containing an acid-labile group. Specific examples of the developer used in this event include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, benzyl formate, phenylethyl formate, methyl 3-phenylpropionate, benzyl propionate, phenylethyl acetate, 2-phenylethyl acetate, etc. One of these organic solvents can be used, or two or more thereof can be used in mixture.

When the development is completed, rinsing is performed. The rinsing liquid is preferably a solvent that is miscible with the developer but does not dissolve the resist film. As such a solvent, it is preferable to use an alcohol having 3 to 10 carbon atoms, an ether compound having 8 to 12 carbon atoms, an alkane, alkene, alkyne, or aromatic solvent, each having 6 to 12 carbon atoms, etc.

Specific examples of the alcohol having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, 1-octanol, etc.

Specific examples of the ether compound having 8 to 12 carbon atoms include di-n-butyl ether, diisobutyl ether, di-sec-butyl ether, di-n-pentyl ether, diisopentyl ether, di-sec-pentyl ether, di-tert-pentyl ether, di-n-hexyl ether, etc.

Specific examples of the alkane having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, cyclononane, etc. Specific examples of the alkene having 6 to 12 carbon atoms include hexene, heptene, octene, cyclohexene, methylcyclohexene, dimethylcyclohexene, cycloheptene, cyclooctene, etc. Specific examples of the alkyne having 6 to 12 carbon atoms include hexyne, heptyne, octyne, etc.

Specific examples of the aromatic solvent include toluene, xylene, ethylbenzene, isopropylbenzene, tert-butylbenzene, mesitylene, etc.

The rinsing can reduce resist pattern collapse and defect formation. Meanwhile, the rinsing is not necessarily essential, and the amount of the solvent used can be reduced by not performing the rinsing.

After the development, a hole pattern or trench pattern can be shrunk by thermal flow, RELACS process, or DSA process. A shrink agent is applied onto the hole pattern, and the shrink agent undergoes crosslinking on the resist film surface by diffusion of the acid catalyst from the resist film during baking, so that the shrink agent is attached to sidewalls of the hole pattern. The baking temperature is preferably 70 to 180° C., more preferably 80 to 170° C. The baking time is preferably 10 to 300 seconds. The extra shrink agent is removed, and thus, the hole pattern is shrunk.

### EXAMPLES

Hereinafter, the present invention will be specifically described with reference to Synthesis Examples, Examples, and Comparative Examples, but the present invention is not limited to the following Examples. Incidentally, the apparatus used was as follows.
MALDI TOF-MS: S3000 manufactured by JEOL Ltd.

### [1] Synthesis of Onium Salt Monomers

### [Example 1-1] Synthesis of Onium Salt Monomer a-1

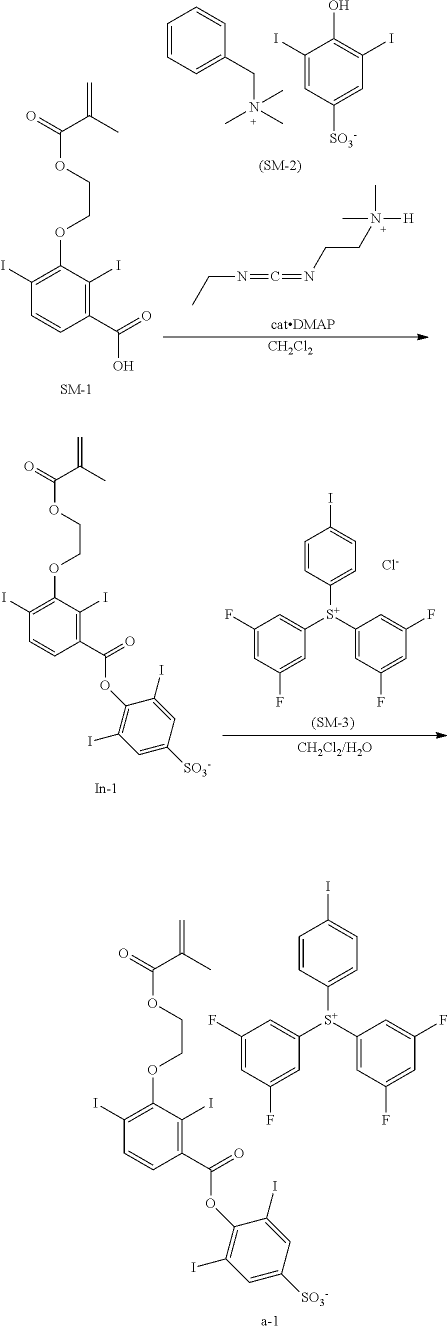

### (1) Synthesis of Intermediate In-1

Under a nitrogen atmosphere, a raw material SM-1 (25.1 g), a raw material SM-2 (31.6 g), DMAP (0.75 g), and methylene chloride (300 g) were added to a reaction container and cooled in an ice bath. While maintaining the temperature inside the reaction container at 20° C. or lower, 1-ethyl-3-(3-dimethylaminopropyl)carbodiimide hydrochloride (13.3 g) was added to the mixture while still in the state of a powder. After the addition, the temperature was raised to room temperature, and the mixture was aged for 12 hours. After the aging, water was added to the reaction product to terminate the reaction, a common aqueous work-up was performed, the solvent was distilled off, and then diisopropyl ether was added to wash the residue. Thus, 51.9 g of intermediate In-1 was obtained as a brown solid (98% yield).

### (2) Synthesis of Onium Salt Monomer a-1

Under a nitrogen atmosphere, the intermediate In-1 (51.9 g), a raw material SM-3 (23.2 g), methylene chloride (200 g), and water (150 g) were added to a reaction container and stirred for 30 minutes. Then, the organic layer was collected, washed with water, and then concentrated under reduced pressure. The concentrated liquid was purified by silica gel chromatography, and diisopropyl ether was added to perform recrystallization. Thus, 61.0 g of onium salt monomer a-1, being the target product, was obtained as a brown crystal (89% yield).

### MALDI TOF-MS:

POSITIVE M⁺461 (C₁₈H₁₀F₄IS⁺ equivalent)
NEGATIVE M⁻909 (C₁₉H₁₃I₄O₈S⁻ equivalent)

### [Examples 1-2 to 1-8] Synthesis of Onium Salt Monomers a-2 to a-8

Onium salt monomers a-2 to a-8, represented by the following formulae, were synthesized using corresponding raw materials and known organic synthesis reactions.

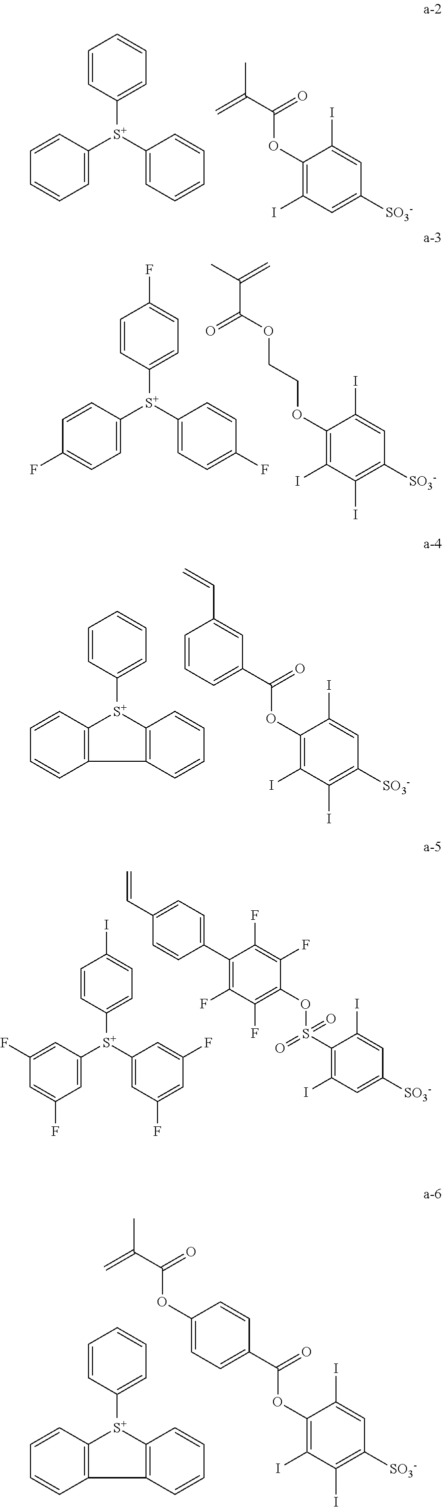

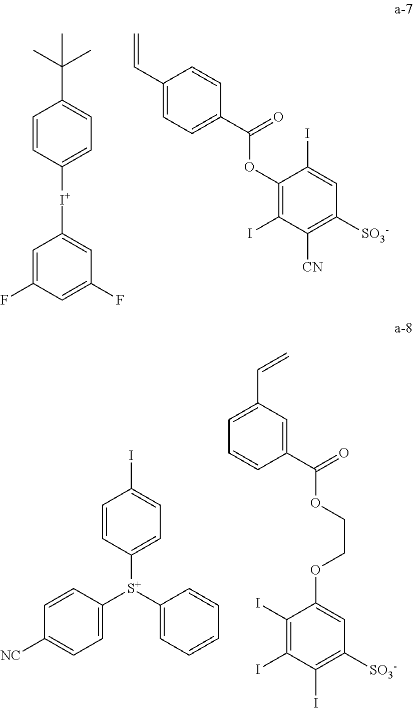

### [Comparative Examples 1-1 to 1-6] Synthesis of Comparative Onium Salt Monomers ca-1 to ca-6

Comparative onium salt monomers ca-1 to ca-6, represented by the following formulae, were synthesized using corresponding raw materials and known organic synthesis reactions.

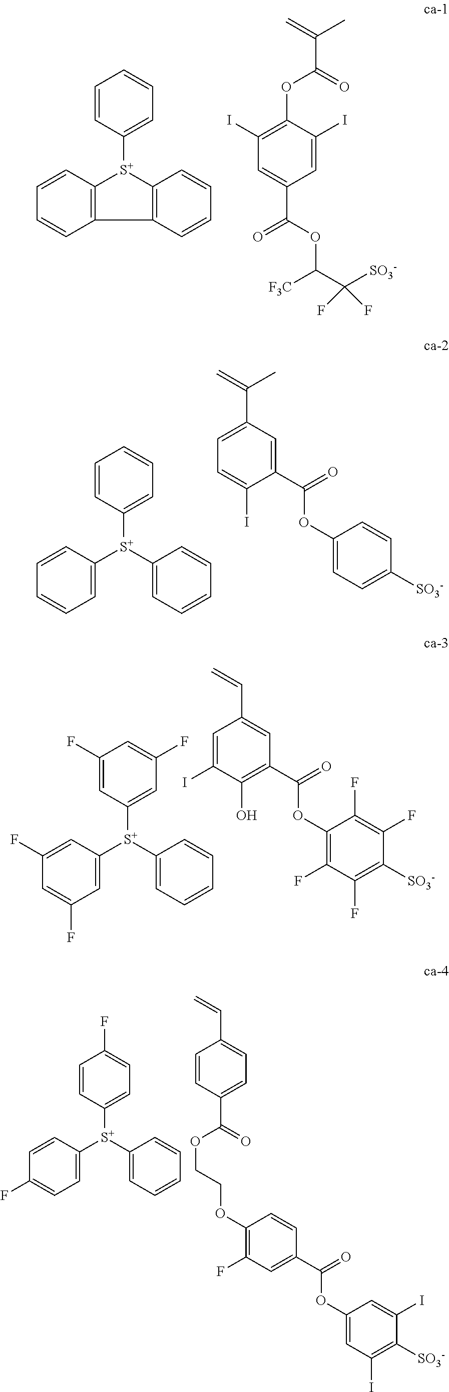

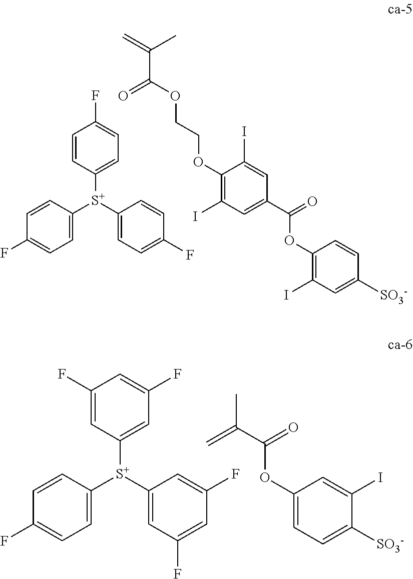

### [2] Synthesis of Base Polymers

Among the monomers used for synthesizing base polymers, monomers other than monomer a-1 to monomer a-8 and comparative monomer ca-1 to comparative monomer ca-6 are as follows.

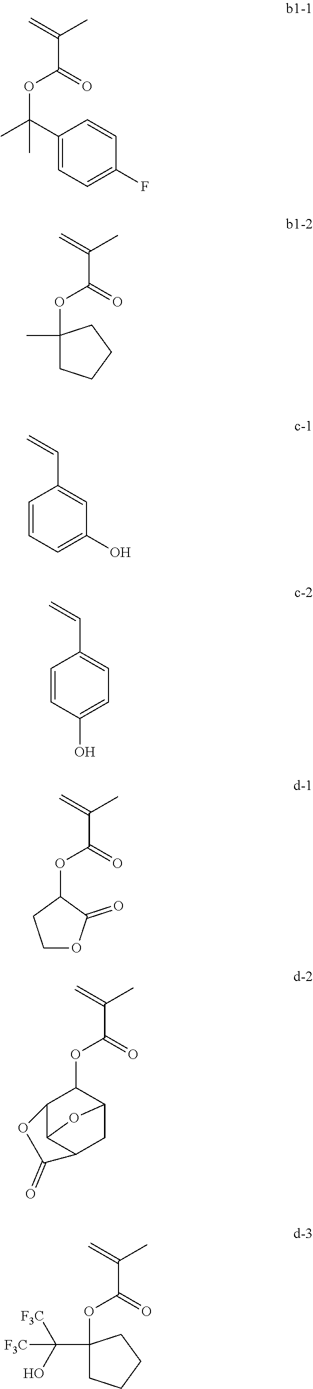

### [Example 2-1] Synthesis of Polymer P-1

Under a nitrogen atmosphere, monomer a-1 (29.7 g), monomer b1-1 (35.6 g), monomer c-1 (10.4 g), 3.34 g of V-601 (manufactured by FUJIFILM Wako Pure Chemical Corporation), and 139 g of MEK were added to a flask to prepare a monomer-polymerization initiator solution. Into another flask with a nitrogen atmosphere, 46 g of MEK was added and heated to 80° C. with stirring, and then the monomer-polymerization initiator solution was added dropwise over 4 hours. After the dropwise addition, the polymerization liquid was further stirred for 2 hours with maintaining the temperature at 80° C., and then cooled to room temperature. The obtained polymerization liquid was added dropwise to 3000 g of vigorously stirred hexane, and a precipitated polymer was filtered. The obtained polymer was washed twice with 600 g of hexane, and then dried in vacuo at 50° C. for 20 hours to obtain a white powder polymer P-1 (68.9 g, 91% yield). The polymer P-1 had Mw of 9200 and Mw/Mn of 1.73. Incidentally, Mw is a value measured in terms of polystyrene, obtained by GPC using DMF as an eluent.

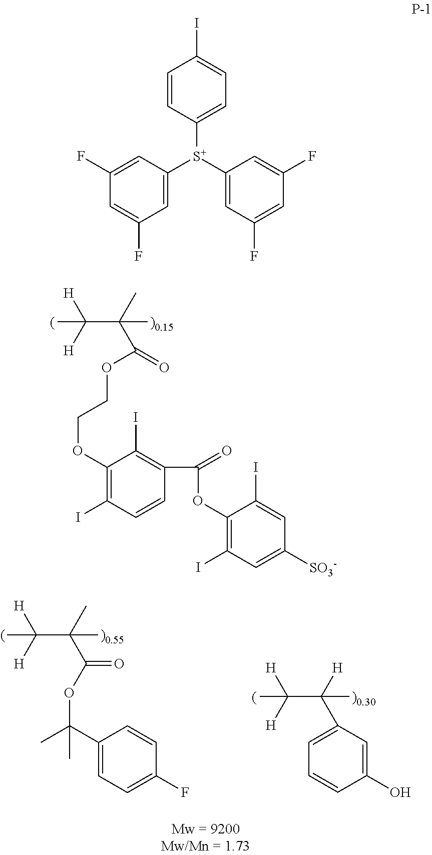

### [Examples 2-2 to 2-15 and Comparative Examples 2-1 to 2-12] Synthesis of Polymers P-2 to P-15 and Comparative Polymers CP-1 to CP-12

The polymers shown in Tables 1 and 2 were produced in the same manner as in Example 2-1, except that the kinds of the monomers and the introduction rates were changed.

TABLE 1

| | | Intro- | | Intro- | | Intro- | | Intro- | | |
|---|---|---|---|---|---|---|---|---|---|---|
| | | duction | | duction | | duction | | duction | | |
| | | rate | | rate | | rate | | rate | | |
| Polymer | Unit 1 | (mol %) | Unit 2 | (mol %) | Unit 3 | (mol %) | Unit 4 | (mol %) | Mw | Mw/Mn |
| P-1 | a-1 | 15 | b1-1 | 55 | c-1 | 30 | — | — | 9200 | 1.73 |
| P-2 | a-2 | 15 | b1-1 | 55 | c-1 | 30 | — | — | 9300 | 1.74 |
| P-3 | a-3 | 15 | b1-1 | 55 | c-1 | 30 | — | — | 9100 | 1.78 |
| P-4 | a-4 | 15 | b1-1 | 55 | c-1 | 30 | — | — | 9400 | 1.74 |
| P-5 | a-5 | 15 | b1-1 | 55 | c-1 | 30 | — | — | 9100 | 1.71 |
| P-6 | a-6 | 15 | b1-1 | 55 | c-1 | 30 | — | — | 9100 | 1.71 |
| P-7 | a-7 | 15 | b1-1 | 55 | c-1 | 30 | — | — | 9100 | 1.69 |
| P-8 | a-8 | 15 | b1-1 | 55 | c-1 | 30 | — | — | 9100 | 1.75 |
| P-9 | a-1 | 15 | b1-2 | 55 | c-1 | 30 | — | — | 9200 | 1.81 |
| P-10 | a-2 | 15 | b1-2 | 55 | c-1 | 30 | — | — | 9300 | 1.71 |
| P-11 | a-1 | 15 | b1-1 | 55 | c-2 | 30 | — | — | 9100 | 1.73 |
| P-12 | a-2 | 10 | b1-1 | 50 | c-1 | 30 | d-1 | 10 | 9200 | 1.66 |
| P-13 | a-2 | 10 | b1-1 | 50 | c-1 | 30 | d-2 | 10 | 9200 | 1.68 |
| P-14 | a-2 | 10 | b1-2 | 50 | c-1 | 30 | d-3 | 10 | 9300 | 1.85 |
| P-15 | a-3 | 12 | b1-1 | 50 | c-1 | 38 | — | — | 8900 | 1.81 |

TABLE 2

| | | Intro- | | Intro- | | Intro- | | Intro- | | Intro- | | |
|---|---|---|---|---|---|---|---|---|---|---|---|---|
| | | duction | | duction | | duction | | duction | | duction | | |
| | | rate | | rate | | rate | | rate | | rate | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | ca-1 | 15 | b1-1 | 55 | c-1 | 30 | — | — | — | — | 9100 | 1.71 |
| CP-2 | ca-2 | 15 | b1-1 | 55 | c-1 | 30 | — | — | — | — | 9100 | 1.72 |
| CP-3 | ca-3 | 15 | b1-1 | 55 | c-1 | 30 | — | — | — | — | 9100 | 1.71 |
| CP-4 | ca-4 | 15 | b1-1 | 55 | c-1 | 30 | — | — | — | — | 9200 | 1.69 |
| CP-5 | ca-1 | 15 | b1-2 | 55 | c-1 | 30 | — | — | — | — | 9500 | 1.69 |
| CP-6 | ca-2 | 15 | b1-1 | 55 | c-2 | 30 | — | — | — | — | 9200 | 1.68 |
| CP-7 | ca-3 | 10 | b1-1 | 50 | c-1 | 30 | d-1 | 10 | — | — | 9200 | 1.75 |
| CP-8 | ca-4 | 10 | b1-1 | 50 | c-1 | 30 | d-2 | 10 | — | — | 9200 | 1.74 |
| CP-9 | ca-1 | 10 | b1-1 | 50 | c-1 | 20 | d-2 | 10 | d-3 | 10 | 9100 | 1.76 |
| CP-10 | ca-2 | 12 | b1-1 | 50 | c-1 | 38 | — | — | — | — | 9000 | 1.75 |
| CP-11 | ca-5 | 15 | b1-1 | 55 | c-1 | 30 | — | — | — | — | 9200 | 1.70 |
| CP-12 | ca-6 | 15 | b1-1 | 55 | c-1 | 30 | — | — | — | — | 9300 | 1.65 |

### [3] Preparation of Chemically-Amplified Resist Compositions

### Examples 3-1 to 3-15 and Comparative Examples 3-1 to 3-12

Prepared was a solution of predetermined components selected from the base polymers (P-1 to P-15) of the present invention, the comparative base polymers (CP-1 to CP-12), acid generators (PAG-1 and PAG-2), and quenchers (SQ-1 to SQ-3 and AQ-1) at the composition shown in the following Tables 3 and 4 by dissolving the components in a solvent containing 0.01 mass % of FC-4430, manufactured by 3M Company, as a surfactant. The solution was filtered with a 0.2-μm Teflon (registered trademark) type filter to prepare chemically-amplified resist compositions (R-1 to R-15 and CR-1 to CR-12).

TABLE 3

| | | | | Acid | | |
|---|---|---|---|---|---|---|
| | | Base polymer | Quencher | generator | Solvent 1 | Solvent 2 |
| | Resist | (parts by | (parts by | (parts by | (parts by | (parts by |
| | composition | mass) | mass) | mass) | mass) | mass) |
| Example 3-1 | R-1 | P-1 (80) | SQ-1 (8.0) | — | PGMEA (2,200) | DAA (900) |
| Example 3-2 | R-2 | P-2 (80) | SQ-1 (8.0) | — | PGMEA (2,200) | DAA (900) |
| Example 3-3 | R-3 | P-3 (80) | SQ-1 (4.0) | — | PGMEA (2,200) | DAA (900) |
| | | | AQ-1 (4.0) | | | |
| Example 3-4 | R-4 | P-4 (80) | SQ-1 (8.0) | — | PGMEA (2,200) | DAA (900) |
| Example 3-5 | R-5 | P-5 (80) | SQ-1 (8.0) | — | PGMEA (2,200) | DAA (900) |
| Example 3-6 | R-6 | P-6 (80) | SQ-1 (8.0) | — | PGMEA (2,200) | DAA (900) |
| Example 3-7 | R-7 | P-7 (80) | SQ-2 (8.0) | — | PGMEA (2,200) | DAA (900) |
| Example 3-8 | R-8 | P-8 (80) | SQ-3 (7.8) | — | PGMEA (2,200) | DAA (900) |
| Example 3-9 | R-9 | P-9 (80) | SQ-1 (8.0) | — | PGMEA (2,200) | DAA (900) |
| Example 3-10 | R-10 | P-10 (80) | SQ-1 (8.0) | PAG-1 (10) | PGMEA (2,200) | DAA (900) |
| Example 3-11 | R-11 | P-11 (80) | SQ-1 (8.0) | PAG-2 (15) | PGMEA (2,200) | DAA (900) |
| Example 3-12 | R-12 | P-12 (80) | SQ-1 (8.0) | — | PGMEA (2,200) | DAA (900) |
| Example 3-13 | R-13 | P-13 (80) | SQ-1 (8.0) | — | PGMEA (2,200) | DAA (900) |
| Example 3-14 | R-14 | P-14 (80) | SQ-2 (8.0) | — | PGMEA (2,200) | DAA (900) |
| Example 3-15 | R-15 | P-15 (80) | SQ-2 (8.0) | — | PGMEA (2,200) | DAA (900) |

TABLE 4

| | | | | Acid | | |
|---|---|---|---|---|---|---|
| | | Base polymer | Quencher | generator | Solvent 1 | Solvent 2 |
| | Resist | (parts by | (parts by | (parts by | (parts by | (parts by |
| | composition | mass) | mass) | mass) | mass) | mass) |
| Comparative | CR-1 | CP-1 (80) | SQ-1 (8.0) | — | PGMEA (2,200) | DAA (900) |
| Example 3-1 | | | | | | |
| Comparative | CR-2 | CP-2 (80) | SQ-1 (8.0) | — | PGMEA (2,200) | DAA (900) |
| Example 3-2 | | | | | | |
| Comparative | CR-3 | CP-3 (80) | SQ-1 (8.0) | — | PGMEA (2,200) | DAA (900) |
| Example 3-3 | | | | | | |
| Comparative | CR-4 | CP-4 (80) | SQ-1 (8.0) | — | PGMEA (2,200) | DAA (900) |
| Example 3-4 | | | | | | |
| Comparative | CR-5 | CP-5 (80) | SQ-2 (4.0) | PAG-1 (10) | PGMEA (2,200) | DAA (900) |
| Example 3-5 | | | AQ-1 (4.0) | | | |
| Comparative | CR-6 | CP-6 (80) | SQ-1 (8.0) | — | PGMEA (2,200) | DAA (900) |
| Example 3-6 | | | | | | |
| Comparative | CR-7 | CP-7 (80) | SQ-1 (8.2) | — | PGMEA (2,200) | DAA (900) |
| Example 3-7 | | | | | | |
| Comparative | CR-8 | CP-8 (80) | SQ-2 (7.8) | — | PGMEA (2,200) | DAA (900) |
| Example 3-8 | | | | | | |
| Comparative | CR-9 | CP-9 (80) | SQ-3 (4.0) | PAG-2 (15) | PGMEA (2,200) | DAA (900) |
| Example 3-9 | | | AQ-1 (4.0) | | | |
| Comparative | CR-10 | CP-10 (80) | SQ-3 (7.8) | — | PGMEA (2,200) | DAA (900) |
| Example 3-10 | | | | | | |
| Comparative | CR-11 | CP-11 (80) | SQ-1 (8.0) | — | PGMEA (2,200) | DAA (900) |
| Example 3-11 | | | | | | |
| Comparative | CR-12 | CP-12 (80) | SQ-1 (8.0) | — | PGMEA (2,200) | DAA (900) |
| Example 3-12 | | | | | | |

In Tables 3 and 4, the solvents, the quenchers (SQ-1 to SQ-3 and AQ-1), and the acid generators (PAG-1 and PAG-2) are as follows.
Solvents:
   PGMEA (propylene glycol monomethyl ether acetate)
   DAA (diacetone alcohol)
Quenchers: SQ-1 to SQ-3 and AQ-1

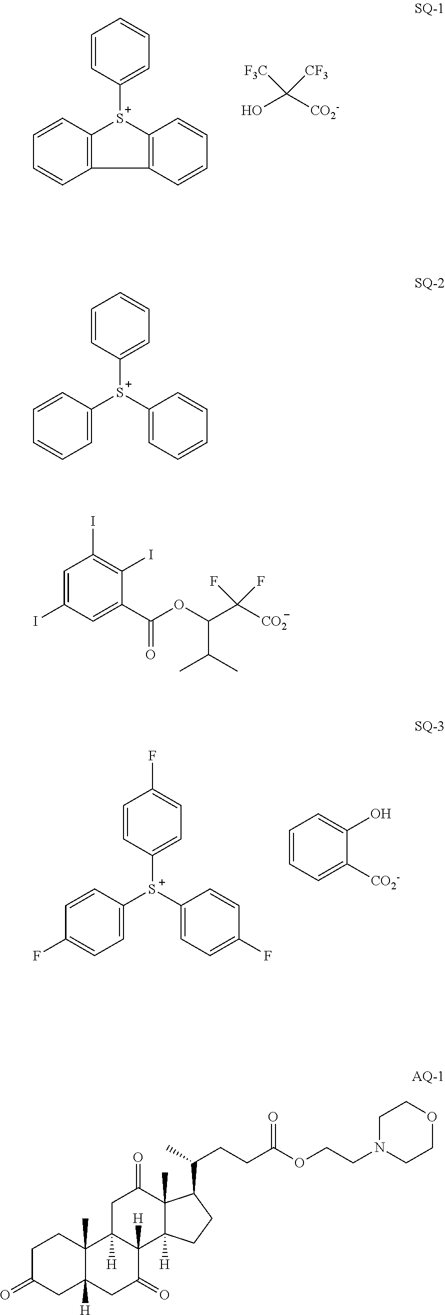

Acid generators: PAG-1 and PAG-2

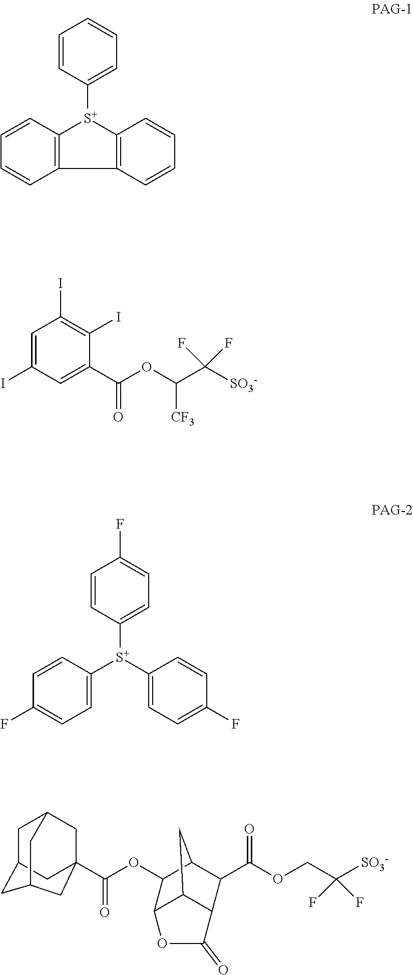

### [4] EUV Lithography Evaluation (1)

### Examples 4-1 to 4-15 and Comparative Examples 4-1 to 4-12

Each of the chemically-amplified resist compositions (R-1 to R-15 and CR-1 to CR-12) shown in Tables 3 and 4 was respectively applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, the substrate was prebaked at 100° C. for 60 seconds using a hot plate to produce each resist film having a film thickness of 50 nm. Each resist film was exposed using an EUV scanner NXE3300 (NA 0.33, σ 0.9/0.6, dipole illumination), manufactured by ASML Holding N.V. The exposure was performed with an LS pattern with 18 nm on wafer size and 36 nm in pitch, and with changing an exposure dose and focus (exposure dose pitch: 1 mJ/cm², focus pitch: 0.020 μm). After the exposure, PEB was performed at a temperature shown in Tables 5 and 6 for 60 seconds. Thereafter, puddle development with a 2.38 mass % aqueous TMAH solution for 30 seconds, rinse with a surfactant-containing rinse material, and spin-drying were performed to obtain a positive pattern.

The obtained LS pattern was observed with a length-measurement SEM (CG6300), manufactured by Hitachi High-Technologies Corporation, to evaluate sensitivity, EL, LWR, DOF, and collapse limit in accordance with the following methods. The results are shown in Tables 5 and 6.

### [Sensitivity Evaluation]

An optimum exposure dose Eop (mJ/cm²) to yield the LS pattern with 18 nm in line width and 36 nm in pitch was determined to specify this value as sensitivity. The smaller this value, the higher the sensitivity.

### [EL Evaluation]

From exposure doses to form the LS pattern within a range of ±10% of 18 nm space width (16.2 to 19.8 nm), EL (unit: %) was determined by the following equation. The larger this value, the better the performance.

EL (%)=(|E₁−E₂|/Eop)×100
E₁: An optimum exposure dose to yield an LS pattern with 16.2 nm in line width and 36 nm in pitch
E₂: An optimum exposure dose to yield an LS pattern with 19.8 nm in line width and 36 nm in pitch
Eop: An optimum exposure dose to yield the LS pattern with 18 nm in line width and 36 nm in pitch

### [LWR Evaluation]

In the LS pattern obtained by irradiation at Eop, sizes in ten positions in the longitudinal direction of the line were measured. From the results, a tripled value (3σ) of a standard variation (σ) was determined as LWR. A smaller LWR value can yield a pattern with smaller roughness and uniform line width.

### [DOF Evaluation]

As evaluation of depth of focus, determined was a focus range to form the LS pattern within a range of ±10% of 18 nm size (16.2 to 19.8 nm). A larger DOF value indicates wider depth of focus.

### [Collapse Limit Evaluation of Line Pattern]

The line size of the LS pattern for each exposure dose at the optimum focus was measured in ten positions in the longitudinal direction. The thinnest line size obtained without a collapse was obtained as the collapse limit. The smaller the collapse limit value, the better the collapse limit.

TABLE 5

| | | PEB | Optimum | | | | Collapse |
|---|---|---|---|---|---|---|---|
| | Resist | temperature | exposure dose | EL | LWR | DOF | limit |
| | composition | (°C) | (mJ/cm²) | (%) | (nm) | (nm) | (nm) |
| Example 4-1 | R-1 | 100 | 36 | 20 | 2.5 | 120 | 10.5 |
| Example 4-2 | R-2 | 100 | 36 | 18 | 2.6 | 120 | 10.6 |
| Example 4-3 | R-3 | 100 | 35 | 16 | 2.5 | 115 | 11.0 |
| Example 4-4 | R-4 | 100 | 34 | 16 | 2.5 | 115 | 11.2 |
| Example 4-5 | R-5 | 100 | 33 | 18 | 2.6 | 120 | 10.8 |
| Example 4-6 | R-6 | 100 | 37 | 16 | 2.4 | 110 | 11.0 |
| Example 4-7 | R-7 | 100 | 35 | 18 | 2.5 | 120 | 10.4 |
| Example 4-8 | R-8 | 100 | 33 | 16 | 2.6 | 115 | 10.8 |
| Example 4-9 | R-9 | 100 | 31 | 19 | 2.4 | 120 | 10.5 |
| Example 4-10 | R-10 | 100 | 35 | 18 | 2.6 | 120 | 10.5 |
| Example 4-11 | R-11 | 100 | 35 | 17 | 2.4 | 120 | 10.6 |
| Example 4-12 | R-12 | 100 | 36 | 18 | 2.6 | 120 | 10.7 |
| Example 4-13 | R-13 | 100 | 35 | 18 | 2.6 | 120 | 10.8 |
| Example 4-14 | R-14 | 100 | 36 | 19 | 2.4 | 120 | 10.5 |
| Example 4-15 | R-15 | 100 | 33 | 17 | 2.7 | 110 | 11.3 |

TABLE 6

| | | PEB | Optimum | | | | Collapse |
|---|---|---|---|---|---|---|---|
| | Resist | temperature | exposure dose | EL | LWR | DOF | limit |
| | composition | (°C) | (mJ/cm²) | (%) | (nm) | (nm) | (nm) |
| Comparative | CR-1 | 100 | 38 | 15 | 3.2 | 100 | 13.1 |
| Example 4-1 | | | | | | | |
| Comparative | CR-2 | 100 | 45 | 12 | 2.8 | 100 | 13.1 |
| Example 4-2 | | | | | | | |
| Comparative | CR-3 | 100 | 38 | 15 | 3.1 | 105 | 13.0 |
| Example 4-3 | | | | | | | |
| Comparative | CR-4 | 100 | 44 | 12 | 2.9 | 95 | 12.8 |
| Example 4-4 | | | | | | | |
| Comparative | CR-5 | 100 | 39 | 15 | 3.0 | 100 | 13.0 |
| Example 4-5 | | | | | | | |
| Comparative | CR-6 | 100 | 48 | 13 | 3.0 | 95 | 12.8 |
| Example 4-6 | | | | | | | |
| Comparative | CR-7 | 100 | 38 | 15 | 3.2 | 105 | 12.8 |
| Example 4-7 | | | | | | | |
| Comparative | CR-8 | 100 | 45 | 14 | 2.9 | 95 | 12.6 |
| Example 4-8 | | | | | | | |
| Comparative | CR-9 | 100 | 39 | 15 | 3.4 | 105 | 12.5 |
| Example 4-9 | | | | | | | |
| Comparative | CR-10 | 100 | 42 | 13 | 2.8 | 100 | 12.6 |
| Example 4-10 | | | | | | | |
| Comparative | CR-11 | 100 | 47 | 14 | 3.0 | 100 | 12.7 |
| Example 4-11 | | | | | | | |
| Comparative | CR-12 | 100 | 50 | 13 | 3.1 | 95 | 13.1 |
| Example 4-12 | | | | | | | |

From the results shown in Tables 5 and 6, it was found that the chemically-amplified resist compositions containing a base polymer including a repeating unit derived from the inventive onium salt monomer had favorable sensitivity and excellent EL, LWR, and DOF. Furthermore, the collapse limit was small, and it was observed that the compositions were resistant to pattern collapse even in fine pattern formation. Thus, it was shown that the inventive chemically-amplified resist composition is suitable as a material for EUV lithography. On the other hand, in the Comparative Examples, where onium salt monomers having anion moieties with structures different from that of the present invention were used, it was not possible to achieve favorable lithography properties.

### [5] EUV Lithography Evaluation (2)

### Examples 5-1 to 5-15 and Comparative Examples 5-1 to 5-12

Each of the chemically-amplified resist compositions (R-1 to R-15 and CR-1 to CR-12) shown in Tables 3 and 4 was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, the substrate was prebaked at 105° C. for 60 seconds using a hot plate to produce a resist film with 50 nm in film thickness. This resist film was exposed using an EUV scanner NXE3400 (NA 0.33, σ 0.9/0.6, quadrupole illumination, 46 nm in pitch on wafer size, hole pattern mask with +20% bias), manufactured by ASML Holding N.V. Then, PEB was performed at a temperature shown in Tables 7 and 8 for 60 seconds using a hot plate. Thereafter, development was performed with a 2.38 mass % aqueous TMAH solution for 30 seconds to form a hole pattern with 23 nm in size.

Using a length-measurement SEM (CG6300), manufactured by Hitachi High-Technologies Corporation, an exposure dose when the hole size was formed with 23 nm was measured to specify this exposure dose as sensitivity. Sizes of fifty holes were measured in this time, and a tripled value (3σ) of a standard variation (σ) calculated from the results was determined as size variation (CDU). Tables 7 and 8 show the results.

TABLE 7

| | | | Optimum | |
|---|---|---|---|---|
| | | PEB | exposure | |
| | Resist | temperature | dose | CDU |
| | composition | (°C) | (mJ/cm²) | (nm) |
| Example 5-1 | R-1 | 95 | 26 | 2.4 |
| Example 5-2 | R-2 | 95 | 26 | 2.1 |
| Example 5-3 | R-3 | 95 | 25 | 2.4 |
| Example 5-4 | R-4 | 95 | 24 | 2.3 |
| Example 5-5 | R-5 | 95 | 25 | 2.4 |
| Example 5-6 | R-6 | 95 | 26 | 2.1 |
| Example 5-7 | R-7 | 95 | 26 | 2.5 |
| Example 5-8 | R-8 | 95 | 25 | 2.0 |
| Example 5-9 | R-9 | 90 | 25 | 2.8 |
| Example 5-10 | R-10 | 90 | 25 | 2.6 |
| Example 5-11 | R-11 | 90 | 24 | 2.5 |
| Example 5-12 | R-12 | 90 | 26 | 2.6 |
| Example 5-13 | R-13 | 90 | 23 | 2.4 |
| Example 5-14 | R-14 | 90 | 26 | 2.5 |
| Example 5-15 | R-15 | 90 | 25 | 2.4 |

TABLE 8

| | | | Optimum | |
|---|---|---|---|---|
| | | PEB | exposure | |
| | Resist | temperature | dose | CDU |
| | composition | (°C) | (mJ/cm²) | (nm) |
| Comparative | CR-1 | 95 | 29 | 2.9 |
| Example 5-1 | | | | |
| Comparative | CR-2 | 95 | 35 | 2.9 |
| Example 5-2 | | | | |
| Comparative | CR-3 | 95 | 28 | 3.0 |
| Example 5-3 | | | | |
| Comparative | CR-4 | 95 | 39 | 3.1 |
| Example 5-4 | | | | |
| Comparative | CR-5 | 95 | 29 | 3.2 |
| Example 5-5 | | | | |
| Comparative | CR-6 | 95 | 35 | 3.1 |
| Example 5-6 | | | | |
| Comparative | CR-7 | 95 | 31 | 3.1 |
| Example 5-7 | | | | |
| Comparative | CR-8 | 95 | 34 | 3.1 |
| Example 5-8 | | | | |
| Comparative | CR-9 | 90 | 29 | 2.9 |
| Example 5-9 | | | | |
| Comparative | CR-10 | 90 | 32 | 3.2 |
| Example 5-10 | | | | |
| Comparative | CR-11 | 95 | 32 | 3.1 |
| Example 5-11 | | | | |
| Comparative | CR-12 | 95 | 36 | 3.2 |
| Example 5-12 | | | | |

From the results shown in Tables 7 and 8, it was confirmed that the inventive chemically-amplified resist compositions had good sensitivity and excellent CDU. On the other hand, in the Comparative Examples, where onium salt monomers having anion moieties with structures different from that of the present invention were used, it was not possible to achieve favorable lithography properties.

The present description includes the following inventions.
[1]: An onium salt monomer represented by the following formula (A),

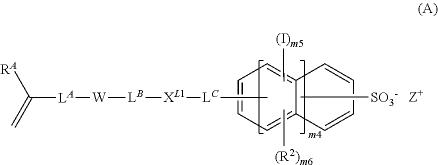

wherein “m4” represents an integer of 0 or 1, “m5” represents an integer of 2 or 3 when “m4” is 0 and represents an integer of 2, 3, 4, or 5 when “m4” is 1, and “m6” represents an integer of 0, 1, or 2 when “m4” is 0 and represents an integer of 0, 1, 2, 3, or 4 when “m4” is 1, provided that when “m4” is 0, 2≤m5+m6≤4 and when “m4” is 1, 2≤m5+m6≤6;
R^{A} represents a hydrogen atom or a methyl group;
R² represents a halogen atom other than an iodine atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m6” is 2 or more, the R²s being identical to or different from each other and the R²s optionally being bonded to each other to form a ring together with the carbon atoms bonded thereto, provided that not both of the two carbon atoms adjacent to the carbon atom having a substituent which is the sulfonic acid “SO₃⁻” are bonded to iodine atoms;
L^{A}, L^{B}, and L^{C} each independently represent a single bond, an ester group, an ether group, an amide group, a sulfonic acid ester group, or a linear, branched, or cyclic alkylene group having 1 to 12 carbon atoms, the alkylene group optionally containing one or more selected from an ester group, an ether group, an amide group, and a sulfonic acid ester group;
X^{L1} represents a single bond or a hydrocarbylene group having 1 to 40 carbon atoms and optionally containing a heteroatom;
W represents a single bond or a hydrocarbylene group having 1 to 30 carbon atoms, the hydrocarbylene group optionally having at least one selected from an oxygen atom, a nitrogen atom, a sulfur atom, and a halogen atom;
Z⁺ represents a sulfonium cation represented by the following formula (Z-1) or an iodonium cation represented by the following formula (Z-2),

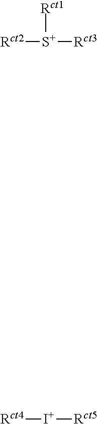

wherein R^{ct1} to R^{ct5} each independently represent a halogen atom or a hydrocarbyl group having 1 to 30 carbon atoms and optionally containing a heteroatom, the
R^{ct1} and the R^{ct2} optionally being bonded to each other to form a ring together with the sulfur atom bonded thereto.
[2]: The onium salt monomer according to [1], represented by the following formula (A1),

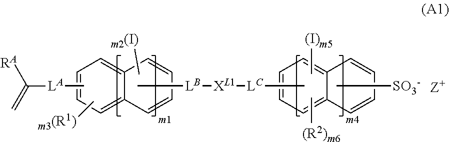

wherein “m1” represents an integer of 0 or 1, “m2” represents an integer of 0, 1, 2, or 3 when “m1” is 0 and represents an integer of 0, 1, 2, 3, 4, or 5 when “m1” is 1, and “m3” represents an integer of 0, 1, 2, 3, or 4 when “m1” is 0 and represents an integer of 0, 1, 2, 3, 4, 5, or 6 when “m1” is 1, provided that when “m1” is 0, 0≤m2+m3≤4 and when “m1” is 1, 0≤m2+m3≤6;
R¹ represents a halogen atom other than an iodine atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m3” is 2 or more, the R's being identical to or different from each other and the R's optionally being bonded to each other to form a ring together with the carbon atoms bonded thereto; and
“m4” to “m6”, R^{A}, R², X^{L1}, L^{A}, L^{B}, L^{C}, and Z⁺ are as defined above.
[3]: The onium salt monomer according to [2], represented by the following formula (A2),

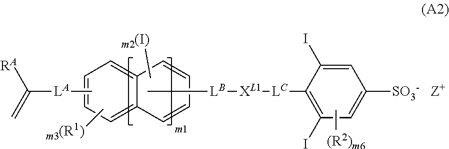

wherein “m1” to “m3”, “m6”, R^{A}, R¹, R², X^{L1}, L^{A}, L^{B}, L^{C}, and Z⁺ are as defined above.
[4]: A monomeric photo-acid generator comprising the onium salt monomer according to any one of [1] to [3].
[5]: A polymer comprising a repeating unit derived from the monomeric photo-acid generator according to [4].
[6]: The polymer according to [5], further comprising a repeating unit represented by the following formula (b1) or (b2),

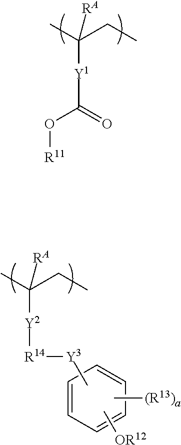

wherein each R^{A} independently represents a hydrogen atom or a methyl group;
Y¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally having at least one selected from a hydroxy group, a halogen atom, a saturated hydrocarbyloxy group having 1 to 8 carbon atoms, and a saturated hydrocarbylcarbonyloxy group having 2 to 8 carbon atoms;
Y² represents a single bond or an ester bond;
Y³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.
[7]: A chemically-amplified resist composition comprising the polymer according to [5] or [6] as a base polymer.
[8]: The chemically-amplified resist composition according to [7], further comprising at least one kind selected from an organic solvent, a quencher, an acid generator, a surfactant, and a dissolution inhibitor.
[9]: A patterning process comprising the steps of:
   forming a resist film on a substrate by using the chemically-amplified resist composition according to [7] or [8];
   exposing the resist film by using a high-energy beam; and
   developing the exposed resist film by using a developer.
[10]: The patterning process according to [9], wherein the high-energy beam is an ArF excimer laser beam having a wavelength of 193 nm, a KrF excimer laser beam having a wavelength of 248 nm, an electron beam, or an extreme ultraviolet ray having a wavelength of 3 to 15 nm.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. An onium salt monomer represented by the following formula (A), 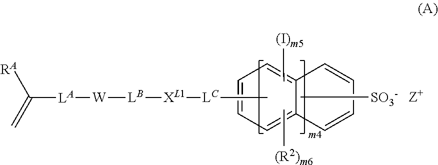
wherein “m4” represents an integer of 0 or 1, “m5” represents an integer of 2 or 3 when “m4” is 0 and represents an integer of 2, 3, 4, or 5 when “m4” is 1, and “m6” represents an integer of 0, 1, or 2 when “m4” is 0 and represents an integer of 0, 1, 2, 3, or 4 when “m4” is 1, provided that when “m4” is 0, 2≤m5+m6≤4 and when “m4” is 1, 2≤m5+m6≤6;
R^{A} represents a hydrogen atom or a methyl group;
R² represents a halogen atom other than an iodine atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m6” is 2 or more, the R²s being identical to or different from each other and the R²s optionally being bonded to each other to form a ring together with the carbon atoms bonded thereto, provided that not both of the two carbon atoms adjacent to the carbon atom having a substituent which is the sulfonic acid “SO₃⁻” are bonded to iodine atoms;
L^{A}, L^{B}, and L^{C} each independently represent a single bond, an ester group, an ether group, an amide group, a sulfonic acid ester group, or a linear, branched, or cyclic alkylene group having 1 to 12 carbon atoms, the alkylene group optionally containing one or more selected from an ester group, an ether group, an amide group, and a sulfonic acid ester group;
X^{L1} represents a single bond or a hydrocarbylene group having 1 to 40 carbon atoms and optionally containing a heteroatom;
W represents a single bond or a hydrocarbylene group having 1 to 30 carbon atoms, the hydrocarbylene group optionally having at least one selected from an oxygen atom, a nitrogen atom, a sulfur atom, and a halogen atom;
Z⁺ represents a sulfonium cation represented by the following formula (Z-1) or an iodonium cation represented by the following formula (Z-2), 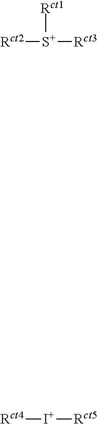
wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.

2. The onium salt monomer according to claim 1, represented by the following formula (A1), 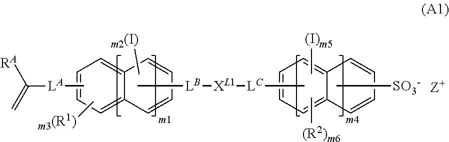
wherein “m1” represents an integer of 0 or 1, “m2” represents an integer of 0, 1, 2, or 3 when “m1” is 0 and represents an integer of 0, 1, 2, 3, 4, or 5 when “m1” is 1, and “m3” represents an integer of 0, 1, 2, 3, or 4 when “m1” is 0 and represents an integer of 0, 1, 2, 3, 4, 5, or 6 when “m1” is 1, provided that when “m1” is 0, 0≤m2+m3≤4 and when “m1” is 1, 0≤m2+m3≤6;
R¹ represents a halogen atom other than an iodine atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m3” is 2 or more, the R's being identical to or different from each other and the R's optionally being bonded to each other to form a ring together with the carbon atoms bonded thereto; and
“m4” to “m6”, R^{A}, R², X^{L1}, L^{A}, L^{B}, L^{C}, and Z⁺ are as defined above.

3. The onium salt monomer according to claim 2, represented by the following formula (A2), 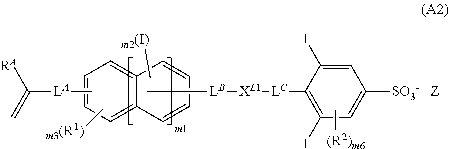
wherein “m1” to “m3”, “m6”, R^{A}, R¹, R², X^{L1}, L^{A}, L^{B}, L^{C}, and Z⁺ are as defined above.

4. A monomeric photo-acid generator comprising the onium salt monomer according to claim 1.

5. A monomeric photo-acid generator comprising the onium salt monomer according to claim 2.

6. A monomeric photo-acid generator comprising the onium salt monomer according to claim 3.

7. A polymer comprising a repeating unit derived from the monomeric photo-acid generator according to claim 4.

8. A polymer comprising a repeating unit derived from the monomeric photo-acid generator according to claim 5.

9. A polymer comprising a repeating unit derived from the monomeric photo-acid generator according to claim 6.

10. The polymer according to claim 7, further comprising a repeating unit represented by the following formula (b1) or (b2), 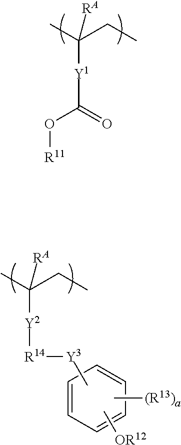
wherein each R^{A} independently represents a hydrogen atom or a methyl group;
Y¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally having at least one selected from a hydroxy group, a halogen atom, a saturated hydrocarbyloxy group having 1 to 8 carbon atoms, and a saturated hydrocarbylcarbonyloxy group having 2 to 8 carbon atoms;
Y² represents a single bond or an ester bond;
Y³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.

11. The polymer according to claim 8, further comprising a repeating unit represented by the following formula (b1) or (b2), 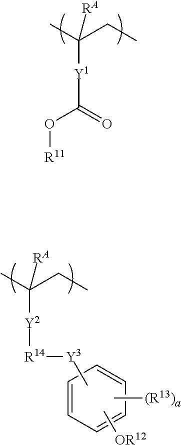
wherein each R^{A} independently represents a hydrogen atom or a methyl group;
Y¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally having at least one selected from a hydroxy group, a halogen atom, a saturated hydrocarbyloxy group having 1 to 8 carbon atoms, and a saturated hydrocarbylcarbonyloxy group having 2 to 8 carbon atoms;
Y² represents a single bond or an ester bond;
Y³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.

12. The polymer according to claim 9, further comprising a repeating unit represented by the following formula (b1) or (b2), 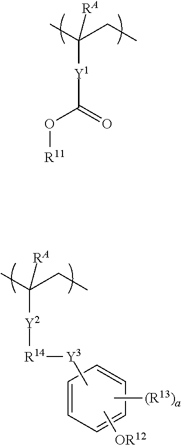
wherein each R^{A} independently represents a hydrogen atom or a methyl group;
Y¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally having at least one selected from a hydroxy group, a halogen atom, a saturated hydrocarbyloxy group having 1 to 8 carbon atoms, and a saturated hydrocarbylcarbonyloxy group having 2 to 8 carbon atoms;
Y² represents a single bond or an ester bond;
Y³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.

13. A chemically-amplified resist composition comprising the polymer according to claim 10 as a base polymer.

14. A chemically-amplified resist composition comprising the polymer according to claim 11 as a base polymer.

15. A chemically-amplified resist composition comprising the polymer according to claim 12 as a base polymer.

16. The chemically-amplified resist composition according to claim 13, further comprising at least one kind selected from an organic solvent, a quencher, an acid generator, a surfactant, and a dissolution inhibitor.

17. The chemically-amplified resist composition according to claim 14, further comprising at least one kind selected from an organic solvent, a quencher, an acid generator, a surfactant, and a dissolution inhibitor.

18. The chemically-amplified resist composition according to claim 15, further comprising at least one kind selected from an organic solvent, a quencher, an acid generator, a surfactant, and a dissolution inhibitor.

19. A patterning process comprising the steps of:
forming a resist film on a substrate by using the chemically-amplified resist composition according to claim 13;
exposing the resist film by using a high-energy beam; and
developing the exposed resist film by using a developer.

20. The patterning process according to claim 19, wherein the high-energy beam is an ArF excimer laser beam having a wavelength of 193 nm, a KrF excimer laser beam having a wavelength of 248 nm, an electron beam, or an extreme ultraviolet ray having a wavelength of 3 to 15 nm.
